(19) United States
(12) Patent Application Publication
(10) Pub. No.: US 2024/0058340 A1
(43) Pub. Date: Feb. 22, 2024

(54) COMPOSITIONS AND METHODS FOR TREATING CANCER

(71) Applicants: The Regents of the University of California, Oakland, CA (US); Erasca, Inc., San Diego, AA (US)

(72) Inventors: Jean-Michel Vernier, San Diego, CA (US); David A. Nathanson, Los Angeles, CA (US); Michael E. Jung, Los Angeles, CA (US); Timothy F. Cloughesy, Calabasas, CA (US); Lorenz Urner, Los Angeles, CA (US); Peter M. Clark, Los Angeles, CA (US); Jonathan Tsang, Los Angeles, CA (US)

(21) Appl. No.: 18/027,313

(22) PCT Filed: Sep. 20, 2021
(86) PCT No.: PCT/US21/51024

§ 371 Date: Mar. 20, 2023

Related U.S. Application Data
(60) Provisional application No. 63/081,239, filed on Sep. 21, 2020.

(51) Int. Cl. A61K 31/519; A61K 31/5377; A61K 31/551; A61K 45/06; A61P 35/00; C07D 491/056; C07D 519/00; G01N 33/66

(52) U.S. Cl. CPC A61K 31/519; A61K 31/5377; A61K 31/551; A61K 45/06; A61P 35/00; C07D 491/056; C07D 519/00; G01N 33/66; C07B 2200/05; G01N 2800/52

(57) ABSTRACT

The present disclosure relates to compounds that are capable penetrating to the blood brain barrier to modulate the activity of EGFR tyrosine kinase. The disclosure further relates to methods of treating glioblastoma and other EGFR mediated cancers. The disclosure further relates to methods of treating glioblastoma and other EGFR mediated cancers that have been determined to have altered glucose metabolism in the presence of inhibitors. The present disclosure also provides methods of administering to a subject a glucose metabolism inhibitor and a cytoplasmic p53 stabilizer.

### RELATED APPLICATIONS

This application claims the benefit of U.S. Provisional Application No. 63/081,239, filed Sep. 21, 2020, the contents of which are fully incorporated by reference herein.

### STATEMENT OF GOVERNMENT SUPPORT

This invention was made with government support under Grant Numbers CA151819, CA211015, CA213133, awarded by the National Institutes of Health. The government has certain rights in the invention.

### BACKGROUND

Glioblastoma (glioblastoma multiforme; GBM) accounts for the majority of primary malignant brain tumors in adults. Amplification and mutation of the epidermal growth factor receptor (EGFR) gene is a signature genetic abnormality encountered in GBM (Sugawa, et al. (1990) Proc. Natl. Acad. Sci. 87: 8602-8606; Ekstrand, et al. (1992) Proc. Natl. Acad. Sci. 89: 4309-4313). A range of potential therapies that target EGFR or its mutant constitutively active form, ΔEGFR, including tyrosine kinase inhibitors (TKIs), monoclonal antibodies, vaccines, and RNA-based agents, are currently in development or in clinical trials for the treatment of GBM. However, to date their efficacy in the clinic has so far been limited by both upfront and acquired drug resistance (Taylor, et al. (2012) Curr. Cancer Drug Targets. 12:197-209). A major limitation is that current therapies such as erlotinib, lapatinib, gefitinib and afatinib are poorly brain penetrant (Razier, et al. (2010) Neuro-Oncology 12:95-103; Reardon, et al. (2015) Neuro-Oncology 17:430-439; Thiessen, et al. (2010) Cancer Chemother. Pharmacol. 65:353-361).

Molecular targeted therapies have revolutionized cancer treatment and paved the path for modern precision medicine. However, despite well-defined actionable genetic alterations, targeted drugs have failed in glioblastoma (GBM) patients. This is in large part due to insufficient CNS penetration of most targeted agents to levels necessary for tumor kill; potentially evoking robust adaptive mechanisms to drive therapeutic resistance. While drug combinations that inhibit both the primary lesion and the compensatory signaling pathway(s) are appealing, these combination therapy strategies have been hampered by enhanced toxicities leading to subthreshold dosing of each drug.

An alternative therapeutic approach targets an oncogenic driver to modify an important functional property for tumor survival, rendering cells vulnerable to an orthogonal second hit. This “synthetic lethal” strategy may be particularly attractive when the oncogene-regulated functional network(s) intersect with tumor cell death pathways. In a certain example, oncogenic signaling drives glucose metabolism to suppress intrinsic apoptosis and promote survival. Inhibition of oncogenic drivers with targeted therapies can trigger the intrinsic apoptotic machinery as a direct consequence of attenuated glucose consumption. The intertwined nature of these tumorigenic pathways may present therapeutic opportunities for rational combination treatments, however, this has yet to be investigated.

In view of the foregoing, there remains a clinical need for brain penetrant chemotherapeutics for the treatment of glioblastoma and other cancers.

### SUMMARY OF THE DISCLOSURE

In one aspect, the present disclosure provides compounds having a structure represented by Formula I:

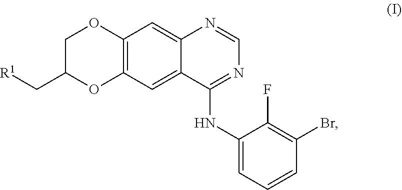

wherein:
R¹ is selected from the group consisting of

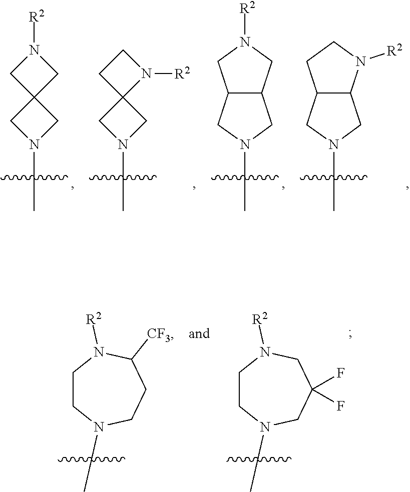

and
R² is selected from C₁-C₆ alkyl and C₃-C₆ cycloalkyl, each of which is optionally substituted with one or more halogen, or a pharmaceutically acceptable salt thereof.

In certain aspects, the present disclosure provides methods of inhibiting EGFR or ΔEGFR, comprising administering to a subject an amount of a compound of the disclosure.

In certain aspects, the present disclosure provides methods of treating cancer comprising administering to a subject in need of a treatment for cancer an amount of a compound of the disclosure. In some embodiments, the cancer is glioblastoma multiforme.

In certain aspects, the present disclosure provides methods of treating cancer comprising administering to a subject a glucose metabolism inhibitor and a cytoplasmic p53 stabilizer, wherein the glucose metabolism inhibitor is a compound of the disclosure. In some embodiments, the cancer is glioblastoma multiforme.

### BRIEF DESCRIPTION OF THE DRAWINGS

FIG. 1A shows the enantiomeric purity of synthetic intermediate 5 as determined by chiral SFC (Chiralpak AD-3 column, 40% MeOH). FIG. 1B shows the enantiomeric purity of synthetic intermediate (S)-5 as determined by chiral SFC (Chiralpak AD-3 column, 40% MeOH). FIG. 1C shows the enantiomeric purity of synthetic intermediate (R)-5 as determined by chiral SFC (Chiralpak AD-3 column, 40% MeOH). FIG. 1D shows the disastereomeric purity of Mosher ester derivatives 5 as determined by chiral SFC (Chiralpak AD-3 column, 40% MeOH).

FIG. 2 depicts the activities of erlotinib, lapatinib, gefitinib, and exemplary compounds of the disclosure against U87 EGFRwt.

FIG. 3 depicts the activities of erlotinib, lapatinib, gefitinib, and exemplary compounds of the disclosure against U87 EGFRvIII.

FIG. 4 depicts the activities of erlotinib, lapatinib, gefitinib, and exemplary compounds of the disclosure against HK301, a patient derived, EGFRvIII mutant GBM gliomasphere.

FIG. 5 depicts the activities of erlotinib, lapatinib, gefitinib, and exemplary compounds of the disclosure against GBM39, a patient derived, EGFRvIII mutant GBM gliomasphere.

FIG. 6 depicts the activities of erlotinib, lapatinib, and exemplary compounds of the disclosure in a GBM39 EGFRvIII mutant mouse model.

FIG. 7A depicts the activities of erlotinib and exemplary compounds of the disclosure in a HCC827 lung cancer EGFR mutant cell line. FIG. 7B depicts the activities of erlotinib and exemplary compounds of the disclosure in a PC9 lung cancer EGFR mutant cell line. FIG. 7C depicts the activities of erlotinib and exemplary compounds of the disclosure in a H838 lung cancer mutant cell line.

FIG. 8 depicts the activities of erlotinib and exemplary compounds of the disclosure in a PC9 lung cancer EGFR mutant mouse model.

FIG. 9 depicts certain metabolites of exemplary compounds of the disclosure.

FIG. 10A depicts the activities of exemplary compounds of the disclosure against HK301.

FIG. 10B depicts the activities of exemplary compounds of the disclosure against GBM39. FIG. 10C depicts the activities of exemplary compounds of the disclosure against NHA.

FIG. 11A decpits the ADME characteristics of an exemplary compound of the disclosure in rats following PO admistration.

FIG. 11B decpits the ADME characteristics of an exemplary compound of the disclosure in rats following PO admistration.

FIG. 12A depicts the activity of certain compounds of the disclosure as compared against the current standard of care (i.e., Labpatinib, Erlotinib, Gefitinib, and AZD3759) against HK301, a patient derived, EGFRvIII mutant GBM gliomasphere.

FIG. 12B depicts the activity of certain compounds of the disclosure as compared against the current standard of care (i.e., Labpatinib, Erlotinib, Gefitinib, and AZD3759) against HK301, a patient derived, EGFRvIII mutant GBM gliomasphere.

FIG. 13A depicts the activity of certain compounds of the disclosure as compared against the current standard of care (i.e., Labpatinib, Erlotinib, Gefitinib, and AZD3759) against GBM39, a patient derived, EGFRvIII mutant GBM gliomasphere. FIG. 13B depicts the activity of certain compounds of the disclosure as compared against the current standard of care (i.e., Labpatinib, Erlotinib, Gefitinib, and AZD3759) against GBM39, a patient derived, EGFRvIII mutant GBM gliomasphere.

FIG. 14A depicts the activity of osimertinib and JGK068S against pEGFRwt. FIG. 14B depicts the activity of osimertinib and JGK068S against pEGFRvIII.

FIG. 15A depicts the activity of osimertinib and JGK068S against HK301. FIG. 15B depicts the activity of osimertinib and JGK068S against GBM39.

FIG. 16A depicts the activity of AZD3759, AZD9291, and JGK068S against certain EGFR mutants. FIG. 16B depicts the activity of AZD3759, AZD9291, and JGK068S against pEGFR A263P. FIG. 16C depicts the activity of AZD3759, AZD9291, and JGK068S against pEGFR A289V. FIG. 16D depicts the activity of AZD3759, AZD9291, and JGK068S against pEGFR A289D. FIG. 16E depicts the activity of AZD3759, AZD9291, and JGK068S against pEGFR G598V.

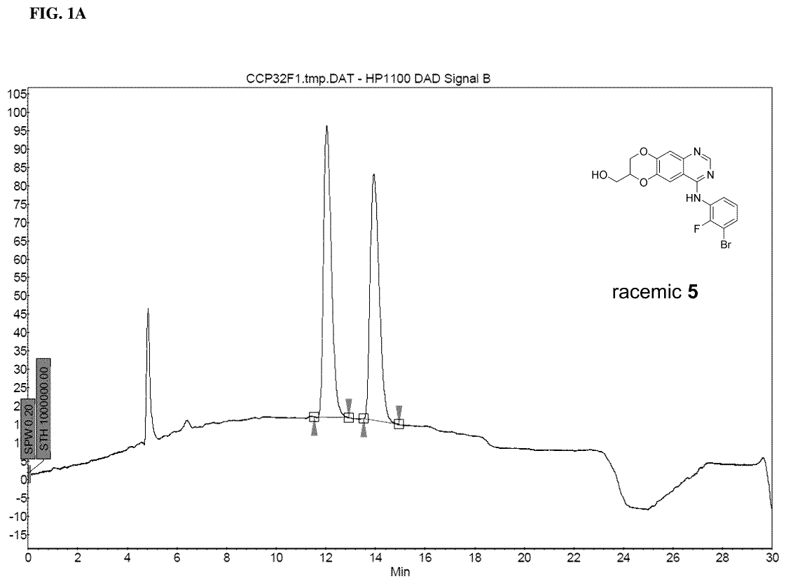

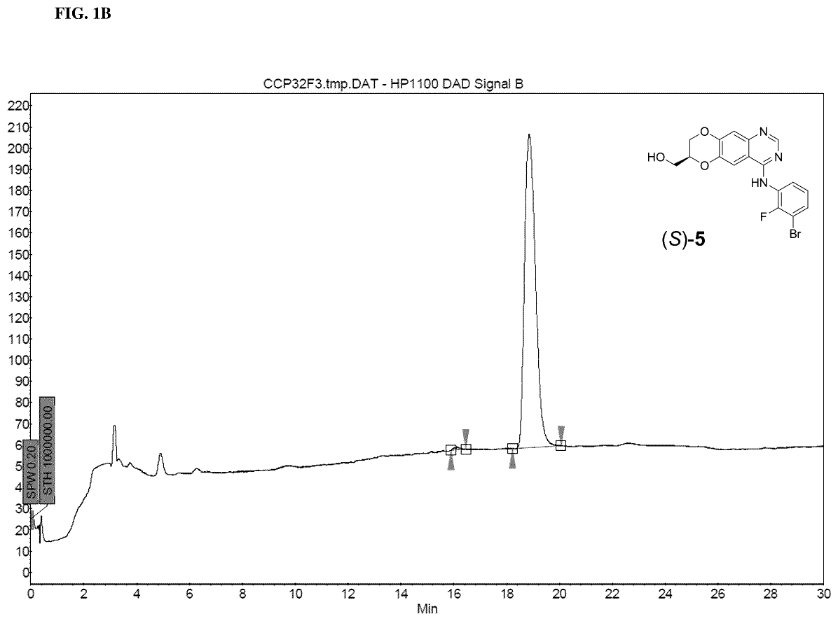

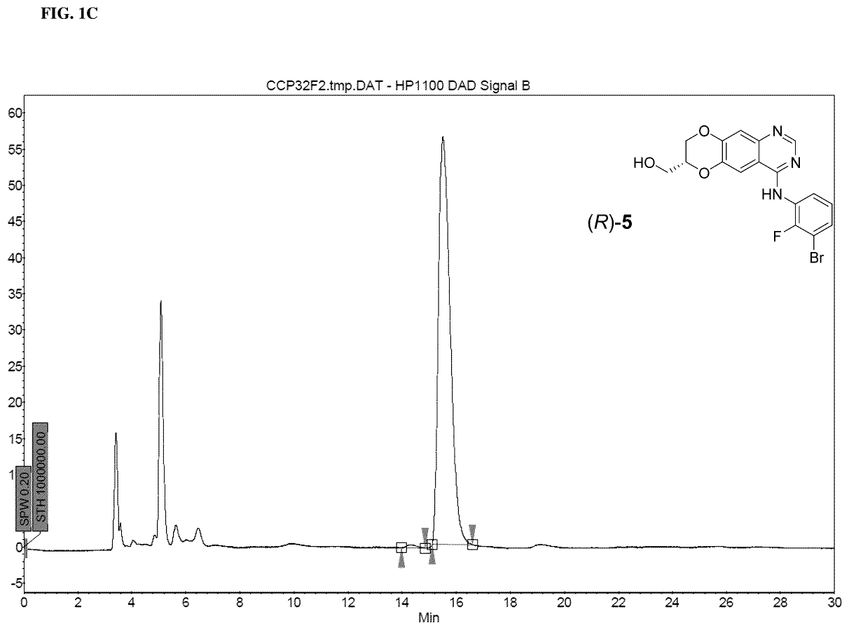

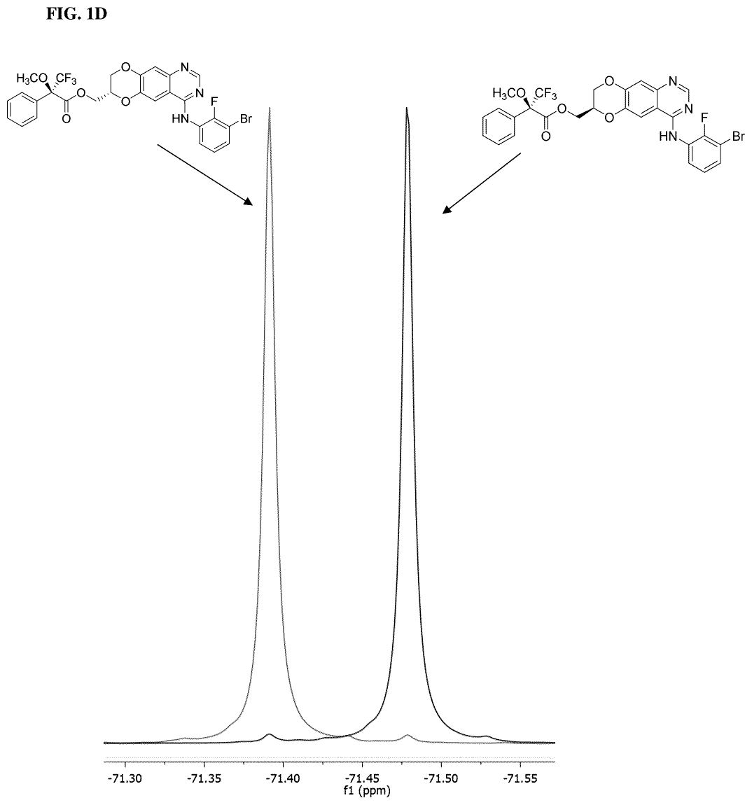

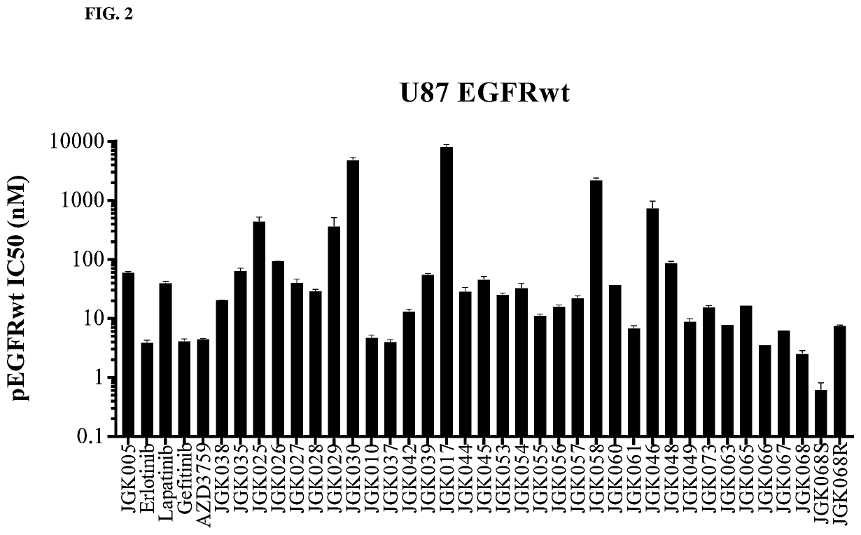

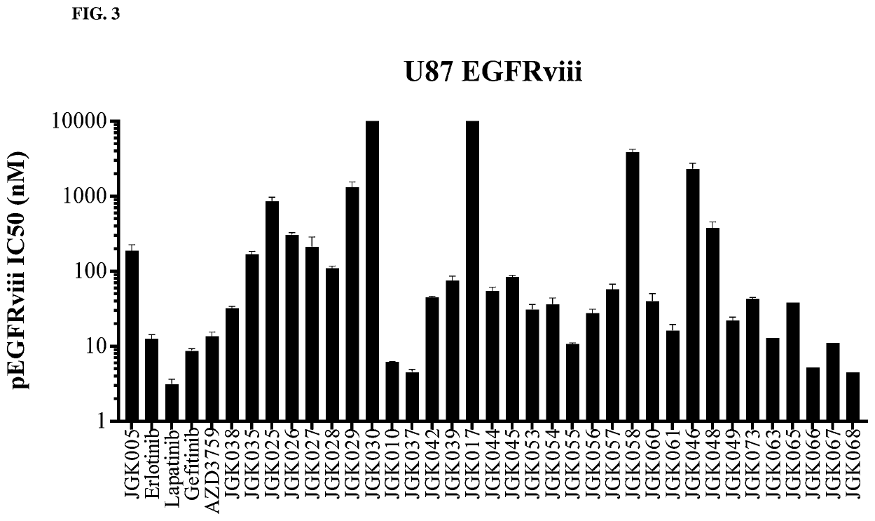

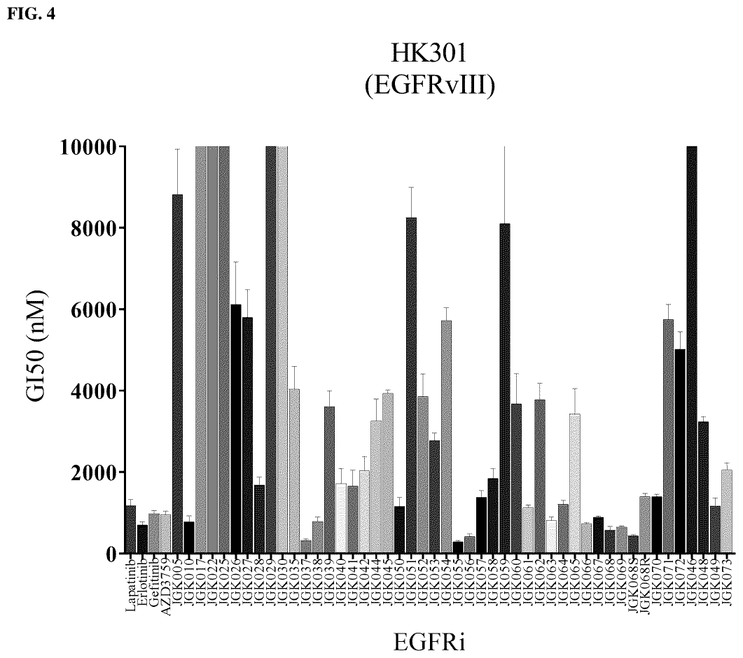

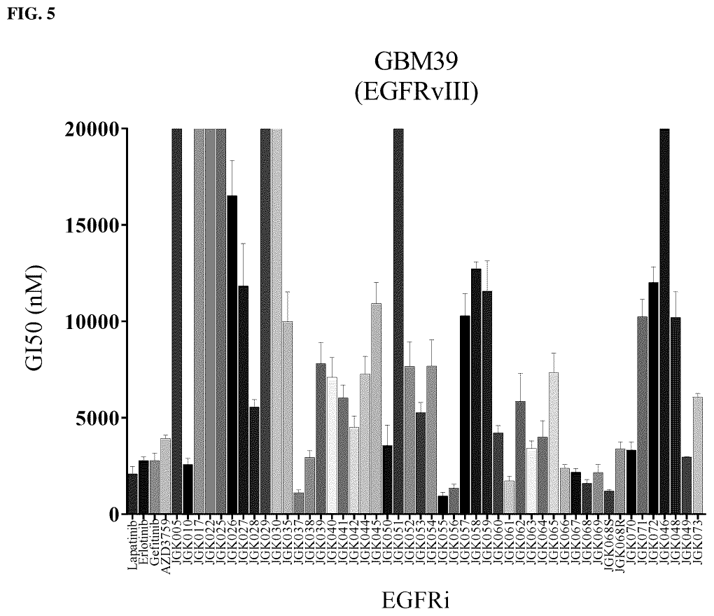

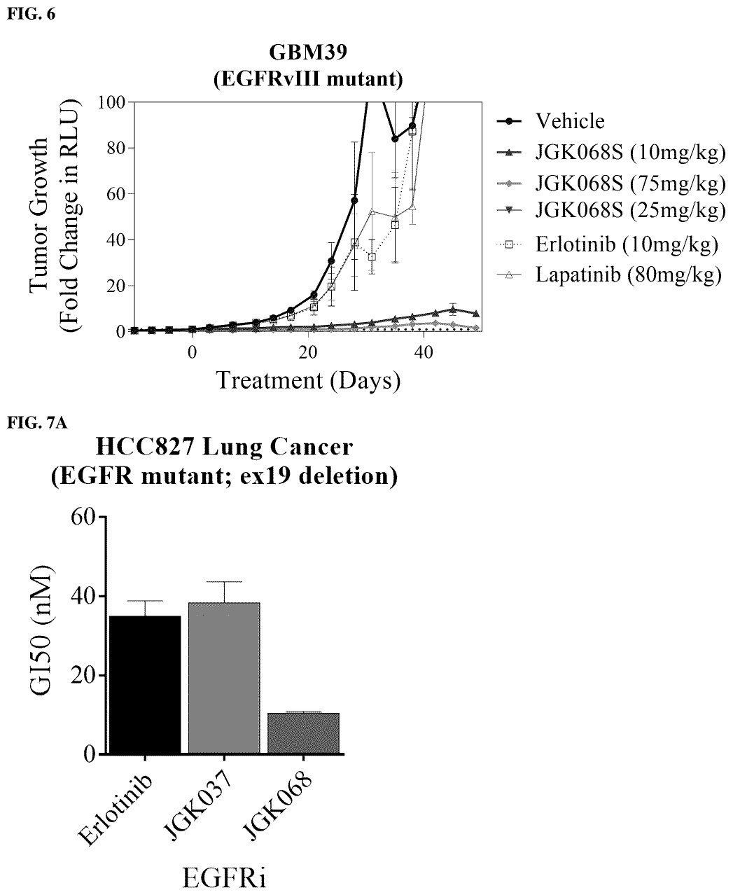

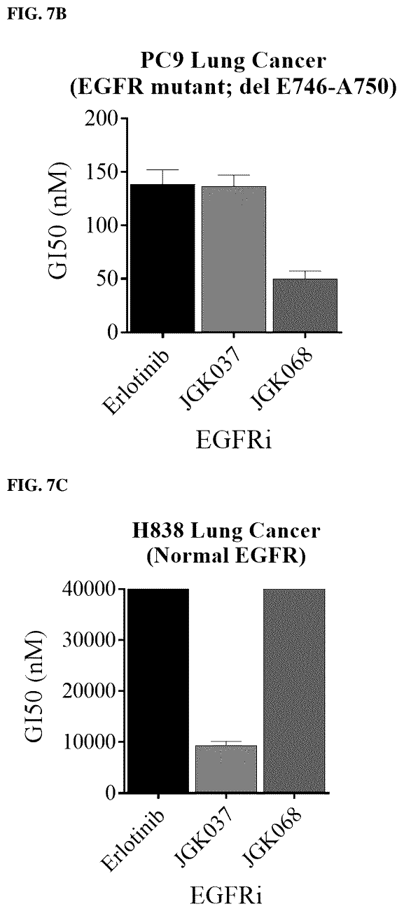

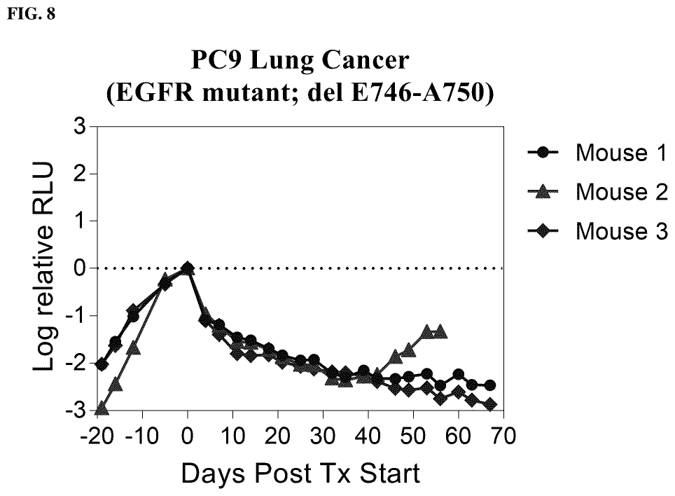

### DETAILED DESCRIPTION OF THE DISCLOSURE

Gliomas are the most commonly occurring form of brain tumor, with glioblastoma multiforme (GBM) being most malignant form, causing 3-4% of all cancer-related deaths (Louis et al. (2007) Acta. Neuropathol. 114: 97-109.). The World Health Organization defines GBM as a grade IV cancer characterized as malignant, mitotically active, and predisposed to necrosis. GBM has a very poor prognosis with a 5-year survival rate of 4-5% with the median survival rate of GBM being 12.6 months (McLendon et al. (2003) Cancer. 98 :1745-1748.). This can be attributed to unique treatment limitations such as a high average age of onset, tumor location, and poor current understandings of the tumor pathophysiology (Louis et al. (2007) Acta. Neuropathol. 114: 97-109).The current standard of care for GBM includes tumor resection with concurrent radiotherapy and chemotherapy and in recent years there have been few marked improvements that increase survival rates (Stewart, et al. (2002) Lancet. 359:1011-1018.).

The standard for GBM chemotherapy is temozolomide (TMZ), which is a brain-penetrant alkylating agent that methylates purines (A or G) in DNA and induces apoptosis (Stupp, et al. (2005) N. Engl. J. Med. 352:987-996). However, TMZ use has drawbacks in that significant risk arises from DNA damage in healthy cells and that GBM cells can rapidly develop resistance towards the drug (Carlsson, et al. (2014) EMBO. Mol. Med. 6: 1359-1370). As such, additional chemotherapy options are urgently required.

EGFR is a member of the HER superfamily of receptor tyrosine kinases together with ERBB2, ERBB3, and ERBB4. A common driver of GBM progression is EGFR amplification, which is found in nearly 40% of all GBM cases (Hynes et al. (2005) Nat. Rev. Cancer. 5: 341-354; Hatanpaa et al. (2010) Neoplasia. 12 :675-684). Additionally, EGFR amplification is associated with the presence of EGFR protein variants: in 68% of EGFR mutants there is a deletion in the N-terminal ligand-binding region between amino acids 6 and 273. These deletions in the ligand-binding domains of EGFR can lead to ligand-independent activation of EGFR (Yamazaki et al. (1990) Jpn. J. Cancer Res. 81: 773-779.).

Thus, there is a need for potent tyrosine kinase inhibitors that have the ability to cross the blood brain barrier and inhibit EGFR and its isoforms.

### Compounds of the Disclosure

In one aspect, the present disclosure provides compounds having a structure represented by Formula (I):

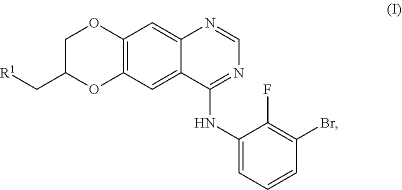

wherein:
R¹ is selected from the group consisting of

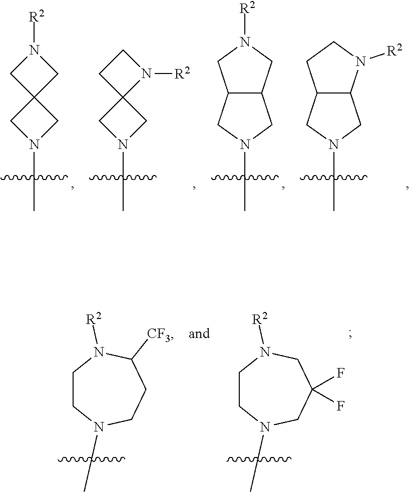

and
R² is selected from C¹-C⁶ alkyl and C³-C⁶ cycloalkyl, each of which is optionally substituted with one or more halogen, or a pharmaceutically acceptable salt thereof.

In certain embodiments of Formula (I), R¹ is

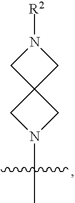

or a pharmaceutically acceptable salt thereof. In other embodiments, R¹ is

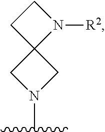

or a pharmaceutically acceptable salt thereof. In yet other embodiments, R¹ is

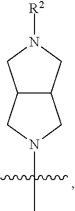

or a pharmaceutically acceptable salt thereof. In yet other embodiments, R¹ is

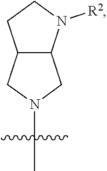

or a pharmaceutically acceptable salt thereof. In yet other embodiments, R¹ is

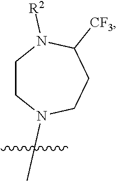

or a pharmaceutically acceptable salt thereof. In yet other embodiments, is

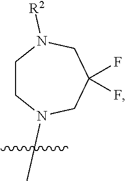

or a pharmaceutically acceptable salt thereof.

In certain embodiments of Formula (I), R² is selected from methyl, ethyl, n-propyl, isopropyl, n-butyl, isobutyl, tert-butyl, pentyl, hexyl, trifluoromethyl, fluoroethyl, and difluoroethyl, or a pharmaceutically acceptable salt thereof. In certain preferred embodiments, R² is selected from methyl, ethyl, n-propyl, isopropyl, tert-butyl, fluoroethyl, and difluoroethyl, or a pharmaceutically acceptable salt thereof. In the most preferred embodiment, R² is methyl, or a pharmaceutically acceptable salt thereof.

In another aspect, the present disclosure provides compounds having a structure represented by Formula (Ia):

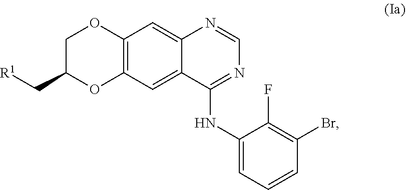

wherein:
R¹ is selected from the group consisting of

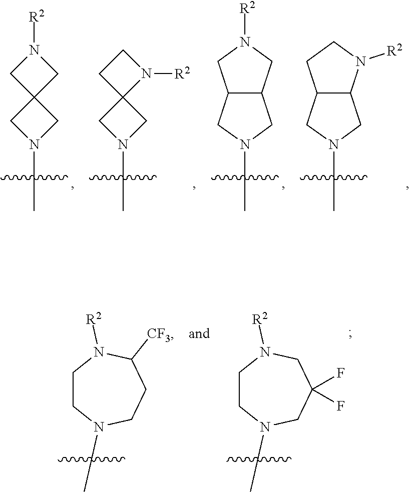

and
R² is selected from C₁-C₆ alkyl and C₃-C₆ cycloalkyl, each of which is optionally substituted with one or more halogen, or a pharmaceutically acceptable salt thereof.

In certain embodiments of Formula (Ia), R¹ is

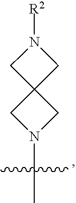

or a pharmaceutically acceptable salt thereof. In other embodiments, R¹ is

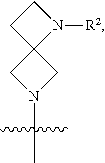

or a pharmaceutically acceptable salt thereof. In yet other embodiments, R¹ is

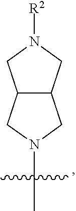

or a pharmaceutically acceptable salt thereof. In yet other embodiments, R¹ is

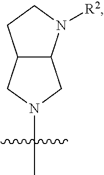

or a pharmaceutically acceptable salt thereof. In yet other embodiments, R¹ is

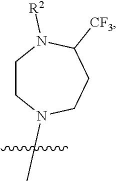

or a pharmaceutically acceptable salt thereof. In yet other embodiments, is

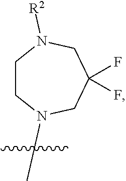

or a pharmaceutically acceptable salt thereof.

In certain embodiments of Formula (Ia), R² is selected from methyl, ethyl, n-propyl, isopropyl, n-butyl, isobutyl, tert-butyl, pentyl, hexyl, trifluoromethyl, fluoroethyl, and difluoroethyl, or a pharmaceutically acceptable salt thereof. In certain preferred embodiments, R² is selected from methyl, ethyl, n-propyl, isopropyl, tert-butyl, fluoroethyl, and difluoroethyl, or a pharmaceutically acceptable salt thereof. In the most preferred embodiment, R² is methyl, or a pharmaceutically acceptable salt thereof.

In yet another aspect, the present disclosure provides compounds having a structure represented by Formula (Ib):

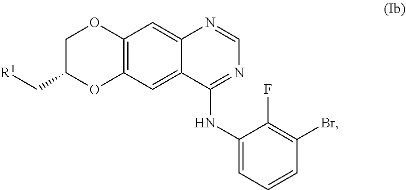

wherein:
R¹ is selected from the group consisting of

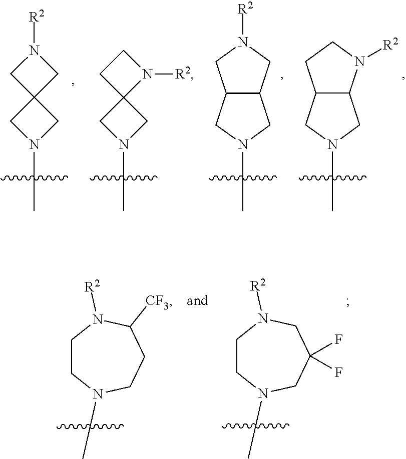

and
R² is selected from C₁-C₆ alkyl and C₃-C₆ cycloalkyl, each of which is optionally substituted with one or more halogen, or a pharmaceutically acceptable salt thereof.

In certain embodiments of Formula (Ib), R¹ is

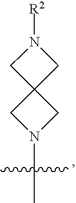

or a pharmaceutically acceptable salt thereof. In other embodiments, R¹ is

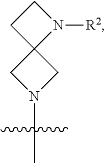

or a pharmaceutically acceptable salt thereof. In yet other embodiments, R¹ is

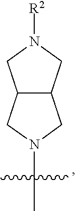

or a pharmaceutically acceptable salt thereof. In yet other embodiments, R¹ is

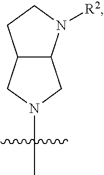

or a pharmaceutically acceptable salt thereof. In yet other embodiments, R¹ is

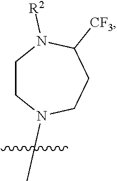

or a pharmaceutically acceptable salt thereof. In yet other embodiments, is

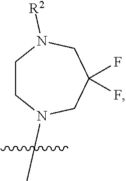

or a pharmaceutically acceptable salt thereof.

In certain embodiments of Formula (Ib), R² is selected from methyl, ethyl, n-propyl, isopropyl, n-butyl, isobutyl, tert-butyl, pentyl, hexyl, trifluoromethyl, fluoroethyl, and difluoroethyl, or a pharmaceutically acceptable salt thereof. In certain preferred embodiments, R² is selected from methyl, ethyl, n-propyl, isopropyl, tert-butyl, fluoroethyl, and difluoroethyl, or a pharmaceutically acceptable salt thereof. In the most preferred embodiment, R² is methyl, or a pharmaceutically acceptable salt thereof.

In cetain embodiments, the compound of Formula (I), (Ia), or (Ib) is enantiomerically enriched.

In cetain embodiments, the compound of Formula (I), (Ia), or (Ib) is diastereomerically enriched.

In cetain embodiments, the compound of Formula (I), (Ia), or (Ib) is in the form of a pharmaceutically acceptable salt. In other embodiemnts, the compound is in the form of a free base.

In certain embodiments, the compound of the disclosure is selected from

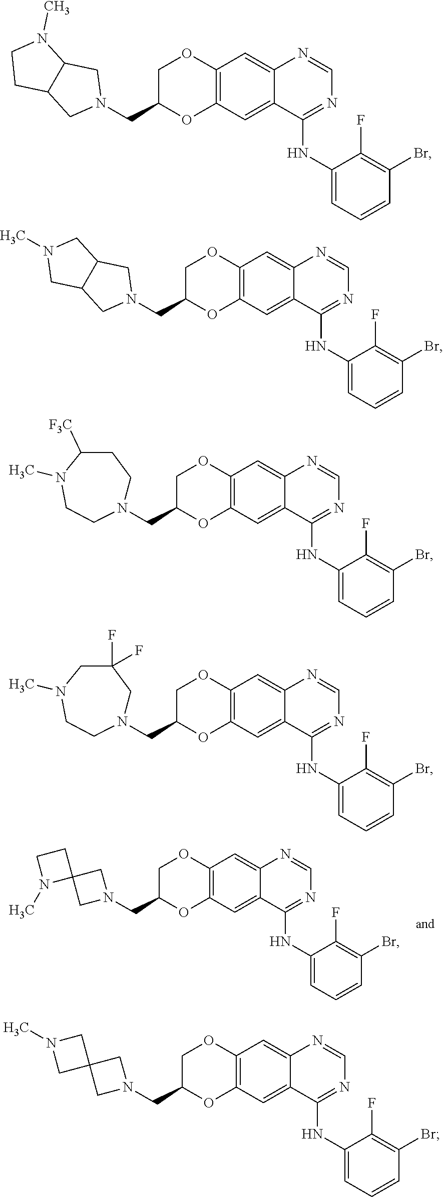 [SMILES: CN1CCC2CN(C[C@H]3COc4cc5ncnc(Nc6cccc(Br)c6F)c5cc4O3)CC21 ; CN1CC2CN(C[C@H]3COc4cc5ncnc(Nc6cccc(Br)c6F)c5cc4O3)CC2C1 ; CN1CCN(C[C@H]2COc3cc4ncnc(Nc5cccc(Br)c5F)c4cc3O2)CCC1C(F)(F)F ; CN1CCN(C[C@H]2COc3cc4ncnc(Nc5cccc(Br)c5F)c4cc3O2)CC(F)(F)C1 ; CN1CCC12CN(C[C@H]1COc3cc4ncnc(Nc5cccc(Br)c5F)c4cc3O1)C2 ; CN1CC2(C1)CN(C[C@H]1COc3cc4ncnc(Nc5cccc(Br)c5F)c4cc3O1)C2]

or a pharmaceutically acceptable salt thereof.

Compounds related to those disclosed herein are disclosed in PCT/US2018/052858, filed Sep. 26, 2020, and PCT/US2020/022743, filed Mar. 13, 2020, the contents of each of which are fully incorporated by reference herein.

### Methods of Treatment

In one aspect, the present disclosure provides methods of treating a disorder or condition in a subject in need thereof by modulation of an epidermal growth factor receptor, the method comprising administering to the subject an amount of a compound or composition of the disclosure, thereby treating the disorder or condition.

In another aspect, the present disclosure provides methods of treating a disorder or condition in a subject in need thereof by antagonizing an epidermal growth factor receptor, the method comprising administering to the subject an amount of a compound or composition of the disclosure, thereby treating the disorder or condition.

In yet another aspect, the present disclosure provides methods of inhibiting EGFR or a variant thereof in a subject, comprising administering to the subject a compound or composition of the disclosure.

In certain embodiments, the EGFR or a variant thereof is ΔEGFR, an ex19 deletion, an EGFRvIII high-expression variant, or one or more EGFR amino acid mutants. In certain embodiments, the one or more EGFR amino acid mutants is selected from L858R, C787S, C797X, L718Q, G724S, S768I, G719X, L792X, G796X, T263P, A289D, A289V, and G598V. In other embodiments, the one or more EGFR amino acid mutants is selected from C797S, G719A, L792H, L792F, L792Y, G796R and G796S.

In yet another aspect, the present disclosure provides methods of treating cancer in a subject, comprising of administering to the subject in need of a treatment for cancer a compound or composition of the disclosure.

In certain embodiments, the cancer is bladder cancer, bone cancer, brain cancer, breast cancer, cardiac cancer, cervical cancer, colon cancer, rectal cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, CNS cancer, non-CNS cancer, or CNS metastases. In certain embodiments, the cancer is lung cancer, colon cancer, rectal cancer, colorectal cancer, esophageal cancer, and pancreatic cancer. In certain embodiments, the cancer is lung cancer. In other embodiments, the cancer is colon cancer. In yet other embodiments, the cancer is rectal cancer. In yet other embodiments, the cancer is colorectal cancer. In yet other embodiments, the cancer is esophageal cancer. In yet other embodiments, the cancer is pancreatic cancer. In certain preferred embodiments, the cancer is glioma, astrocytoma or glioblastoma. In certain embodiments, the cancer is glioma. In other embodiments, the cancer is astrocytoma. In certain embodiments, the astrocytoma is low-grade astrocytoma, mixed oligoastrocytoma, pilocytic astrocytoma, pleomorphic xanthoastrocytoma, subependymal giant cell astrocytoma, or anaplastic astrocytoma. In the most preferred embodiments, the cancer is glioblastoma.

In yet another aspect, the present disclosure provides methods of reducing glioblastoma proliferation in a subject, comprising administering to the subject an amount of a compound or composition of the disclosure. In certain embodiments, the method further comprises administering to the subject a MDM2 inhibitor.

In yet another aspect, the present disclosure provides methods of reducing glioblastoma proliferation in a subject, comprising administering to the subject an effective amount of a compound or composition of the disclosure and a second agent selected from an MDM2 inhibitor, a BCL-xL inhibitor, or a BCL-2 inhibitor, after determining that the glucose metabolism in a sample taken from the subject is susceptible to a glucose metabolism inhibitor.

In yet another aspect, the present disclosure provides methods for treating cancer or reducing cancer cell proliferation in a subject that has been determined to have cancer that is responsive to a glucose metabolism inhibitor, comprising administering to the subject an amount of a compound or composition of the disclosure and a p53 stabilizer.

In yet another aspect, the present disclosure provides methods for treating malignant glioma or glioblastoma in a subject, comprising administering to the subject an amount of a compound or composition of the disclosure and a p53 stabilizer.

In certain embodiments of the methods disclosed herein, the subject has been determined to be susceptible to the glucose metabolism inhibitor by a method comprising:
a. obtaining a tumor biopsy from the subject;
b. measuring the level of glucose uptake by the tumor cells in the presence of the glucose metabolism inhibitor;
c. comparing the level of glucose uptake by the tumor cells obtained in step b. to the level of glucose uptake by a control; and
d. determining that the subject is susceptible to the glucose metabolism inhibitor if the level of glucose uptake by the tumor cells is attenuated compared to the control.

In certain embodiments of the methods disclosed herein, the glucose uptake is measured by the uptake of radio-labelled glucose 2-deoxy-2-[fluorine-18]fluoro-D-glucose (18F-FDG). In certain embodiments, the method further comprises detecting the 18F-FDG by positron emission tomography (PET). In certain embodiments, the reduction in the glucose level between the second blood sample and the first blood sample is about or greater than 0.15 mM. In certain embodiments, the reduction in the glucose level between the second blood sample and the first blood sample is about or greater than 0.20 mM. In certain embodiments, the reduction in the glucose level between the second blood sample and the first blood sample is in the range of 0.15 mM-2.0 mM. In cetain embodiments, the reduction in the glucose level between the second blood sample and the first blood sample is in the range of 0.25 mM-1.0 mM.

In certain embodiments of the methods disclosed herein, the subject has been determined to be susceptible to the glucose metabolism inhibitor by a method comprising:
a. obtaining a first blood sample from the subject;
b. placing the subject on a ketogenic diet;
c. obtaining a second blood sample from the subject after being placed on a ketogenic diet for a period of time;
d. measuring glucose level in the first and in the second blood sample;
e. comparing the glucose level in the second blood sample with the glucose level in the first blood sample; and
f. determining that the subject is susceptible if the glucose level in the second blood sample is reduced as compared to glucose levels in the first blood sample.

In certain embodiments of the methods disclosed herein, the reduction in the glucose level between the second blood sample and the control blood sample is about or greater than 0.15 mM. In certain embodiments, the reduction in the glucose level between the second blood sample and the control blood sample is about or greater than 0.20 mM. In certain embodiments, the reduction in the glucose level between the second blood sample and the control blood sample is in the range of 0.15 mM-2.0 mM. In cetain embodiments, the reduction in the glucose level between the second blood sample and the control blood sample is in the range of 0.25 mM-1.0 mM.

In certain embodiments of the methods disclosed herein, the compound or composition of the disclosure and the p53 stabilizer are administered to the subject in the same composition. In certain embodiments, the compound or composition according of the disclosure and the p53 stabilizer are administered to the subject conjointly. In certain embodiments, the compound or composition of the disclosure and the p53 stabilizer are administered to the subject within 24 hours of each other. In certain embodiments, the compound or composition of the disclosure and the p53 stabilizer are administered to the subject within 6 hours of each other. In certain embodiments, the compound or composition of the disclosure and the p53 stabilizer are administered to the subject within 2 hours of each other. In certain embodiments, the compound or composition of the disclosure and the p53 stabilizer are administered to the subject within 1 hour of each other. In certain embodiments, the compound or composition of the disclosure and the p53 stabilizer are administered to the subject within 30 minutes of each other. In certain embodiments, the compound or composition of the disclosure and the p53 stabilizer are administered to the subject at the same time.

In certain embodiments of the methods disclosed herein, the subject has been diagnosed with glioblastoma multiforme. In certain embodiments, the subject has been previously treated for glioblastoma with a prior treatment. In certain embodiments, the subject has been determined to be resistant to the prior treatment.

In certain embodiments, the methods disclosed herein further comprise administering to the subject of one or more additional therapeutic agents. In certain embodiments, the p53 stabilizer is an MDM2 inhibitor or antagonist. In certain embodiments, the MDM2 inhibitor is a nutlin. In certain embodiments, the MDM2 inhibitor is nutlin-3 or idasanutlin. In certain embodiments, the MDM2 inhibitor is RO5045337, RO5503781, RO6839921, SAR405838, DS-3032, DS-3032b, or AMG-232. In other embodiments, the p53 stabilizer is a BCL-2 inhibitor. In certain embodiments, the BCL-2 inhibitor is antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, or APG-1252. In other embodiments, the p53 stabilizer is a Bcl-xL inhibitor. In certain embodiments, the Bcl-xL inhibitor is WEHI 539, ABT-263, ABT-199, ABT-737, sabutoclax, AT101, TW-37, APG-1252, or gambogic acid.

In certain embodiments, the methods disclosed herein further comprise administering to the subject of one or more additional therapeutic agents. In certain embodiments, the one or more additional therapeutic agents is selected from KRAS G12C inhibitors, EGFR inhibitors, SHP2 inhibitors, CDK4/6 inhibitors, ERK inhibitors, MEK inhibitors, and MET inhibitors. In certain embodiments, the one or more additional therapeutic agents is selected from one or more KRAS G12C inhibitors. In certain embodiments, the one or more KRAS G12C inhibitors is selected from AMG 510, MRTX849, and GDC-6036. In certain embodiments, the one or more KRAS G12C inhibitors is AMG510. In certain embodiments, the one or more KRAS G12C inhibitors is MRTX849. In certain embodiments, the one or more KRAS G12C inhibitors is GDC-6036. In certain embodiments, the one or more additional therapeutic agents is selected from one or more EGFR inhibitors. In certain embodiments, the one or more EGFR inhibitors is selected from osimertinib, afatinib, erlotinib, gefitinib, lazertinib, nazartinib, dacomitinib, BLU-945, icotinib, cetuximab, paninitumab, amivantamab, lapatinib, neratinib, zorifertinib, and mobicertinib. In certain embodimens, the one or more additional therapeutic agents is selected from one or more SHP2 inhibitors. In certain embodiments, the one or more EGFR inhibitors is osimertinib. In certain embodiments, the one or more EGFR inhibitors is afatinib. In certain embodiments, the one or more EGFR inhibitors is erlotinib. In certain embodiments, the one or more EGFR inhibitors is gefitinib. In certain embodiments, the one or more EGFR inhibitors is lazertinib. In certain embodiments, the one or more EGFR inhibitors is nazartinib. In certain embodiments, the one or more EGFR inhibitors is dacomitinib. In certain embodiments, the one or more EGFR inhibitors is BLU-945. In certain embodiments, the one or more EGFR inhibitors is icotinib. In certain embodiments, the one or more EGFR inhibitors is cetuximab. In certain embodiments, the one or more EGFR inhibitors is paninitumab. In certain embodiments, the one or more EGFR inhibitors is amivantamab. In certain embodiments, the one or more EGFR inhibitors is lapatinib. In certain embodiments, the one or more EGFR inhibitors is neratinib. In certain embodiments, the one or more EGFR inhibitors is zorifertinib. In certain embodiments, the one or more EGFR inhibitors is mobicertinib. In certain embodiments, the one or more SHP2 inhibitors is selected from ERAS-601, TNO155, RMC-4630, JAB-3068, JAB-3312, and RLY-1971. In certain embodiments, the one or more SHP2 inhibitors is ERAS-601. In certain embodiments, the one or more SHP2 inhibitors is TNO155. In certain embodiments, one or more SHP2 inhibitors is RMC-4630. In certain embodiments, the one or more SHP2 inhibitors is JAB-3068. In certain embodiments, the one or more SHP2 inhibitors is JAB-3312. In certain embodiments, the one or more SHP2 inhibitors is RLY-1971. In certain embodiments, the one or more additional therapeutic agents is selected from one or more CDK4/6 inhibitors. In certain embodiments, the one or more CDK4/6 inhibitors is selected from palbociclib, abemaciclib, and ribociclib. In certain embodiments, the one or more CDK4/6 inhibitors is palbociclib. In certain embodiments, the one or more CDK4/6 inhibitors is abemaciclib. In certain embodiments, the one or more CDK4/6 inhibitors is ribociclib. In certain embodiments, the one or more additional therapeutic agents is selected from one or more ERK inhibitors. In certain embodiments, the one or more ERK inhibitors is selected from ulixertinib, ASN007, LY3214996, and LTT462. In certain embodiments, the one or more ERK inhibitors is ulixertinib. In certain embodiments, the one or more ERK inhibitors is ASN007. In certain embodiments, the one or more ERK inhibitors is LY3214996. In certain embodiments, he one or more ERK inhibitors is LTT462. In certain embodiments, the one or more additional therapeutic agents is selected from one or more MEK inhibitors. In certain embodiments, the one or more MEK inhibitors is selected from trametinib, binimetinib, cobimetinib, and selumetinib. In certain embodiments, the one or more MEK inhibitors is trametinib. In certain embodiments, the one or more MEK inhibitors is binimetinib. In certain embodiments, the one or more MEK inhibitors is cobimetinib. In certain embodiments, the one or more MEK inhibitors is selumetinib. In certain embodiments, the one or more additional therapeutic agents is selected from one or more MET inhibitors. In certain embodiments, the one or more MET inhibitors is selected from capmatinib, crizotinib, and savolitinib. In certain embodiments, the one or more MET inhibitors is capmatinib. In certain embodiments, the one or more MET inhibitors is crizotinib. In certain embodiments, the one or more MET inhibitors is savolitinib.

In yet another aspect, the present disclosure provides compounds or compositions for use as a medicament. In certain embodiments, the medicament is used in the treatment of cancer in a subject. In certain embodiments, the cancer is bladder cancer, bone cancer, brain cancer, breast cancer, cardiac cancer, cervical cancer, colon cancer, rectal cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, CNS cancer, non-CNS cancer, or CNS metastases. In certain embodiments, the cancer is lung cancer, colon cancer, rectal cancer, colorectal cancer, esophageal cancer, and pancreatic cancer. In certain embodiments, the cancer is lung cancer. In other embodiments, the cancer is colon cancer. In yet other embodiments, the cancer is rectal cancer. In yet other embodiments, the cancer is colorectal cancer. In yet other embodiments, the cancer is esophageal cancer. In yet other embodiments, the cancer is pancreatic cancer. In certain preferred embodiments, the cancer is glioma, astrocytoma or glioblastoma. In certain embodiments, the cancer is glioma. In other embodiments, the cancer is astrocytoma. In certain embodiments, the astrocytoma is low-grade astrocytoma, mixed oligoastrocytoma, pilocytic astrocytoma, pleomorphic xanthoastrocytoma, subependymal giant cell astrocytoma, or anaplastic astrocytoma. In the most preferred embodiments, the cancer is glioblastoma.

### Types and Stages of Gliomas

Primary malignant brain tumors are tumors that start in the brain or spine are known collectively as gliomas. Gliomas are not a specific type of cancer but are a term used to describe tumors that originate in glial cells. Examples of primary malignant brain tumors include astrocytomas, pilocytic astrocytomas, pleomorphic xanthoastrocytomas, diffuse astrocytomas, anaplastic astrocytomas, GBMs, gangliogliomas, oligodendrogliomas, ependymomas. According to the WHO classification of brain tumors, astrocytomas have been categorized into four grades, determined by the underlying pathology. The characteristics that are used to classify gliomas include mitoses, cellular or nuclear atypia, and vascular proliferation and necrosis with pseudopalisading features. Malignant (or high-grade) gliomas include anaplastic glioma (WHO grade III) as well as glioblastoma multiforme (GBM; WHO grade IV). These are the most aggressive brain tumors with the worst prognosis.

GBMs is the most common, complex, treatment resistant, and deadliest type of brain cancer, accounting for 45% of all brain cancers, with nearly 11,000 men, women, and children diagnosed each year. GBM (also known as grade-4 astrocytoma and glioblastoma multiforme) are the most common types of malignant (cancerous) primary brain tumors. They are extremely aggressive for a number of reasons. First, glioblastoma cells multiply quickly, as they secrete substances that stimulate a rich blood supply. They also have an ability to invade and infiltrate long distances into the normal brain by sending microscopic tendrils of tumor alongside normal cells. Two types of glioblastomas are known. Primary GBM are the most common form; they grow quickly and often cause symptoms early. Secondary glioblastomas are less common, accounting for about 10 percent of all GBMs. They progress from low-grade diffuse astrocytoma or anaplastic astrocytoma, and are more often found in younger patients. Secondary GBM are preferentially located in the frontal lobe and carry a better prognosis.

GBM is usually treated by combined multi-modal treatment plan including surgical removal of the tumor, radiation and chemotherapy. First, as much tumor as possible is removed during surgery. The tumor's location in the brain often determines how much of it can be safely removed. After surgery, radiation and chemotherapy slow the growth of remaining tumor cells. The oral chemotherapy drug, temozolomide, is most often used for six weeks, and then monthly thereafter. Another drug, bevacizumab (known as Avastin®), is also used during treatment. This drug attacks the tumor's ability to recruit blood supply, often slowing or even stopping tumor growth.

Novel investigational treatments are also used and these may involve adding treatments to the standard therapy or replacing one part of the standard therapy with a different treatment that may work better. Some of these treatments include immunotherapy such as vaccine immunotherapies, or low-dose pulses of electricity to the area of the brain where the tumor exists and nano therapies involving spherical nucleic acids (SNAs) such as NU-0129. In some embodiments, the methods of the current disclosure are used in combination with one or more of the aforementioned therapies.

### Methods of Assessment

### Glucose Uptake Tests

In embodiments of the methods and compositions of the current disclosure, the subject with GBM or cancer is classified to be either a “metabolic responder” or a “metabolic non-responder” i.e. determined to be susceptible to glucose metabolism inhibitors. In certain embodiments, the classification of the subject is prior to administering to the subject a treatment comprising a glucose metabolism inhibitor and a cytoplasmic p53 stabilizer. Accordingly, the current disclosure provides for methods for assessing a cancer, classifying a subject, determining the susceptibility of a subject to treatments involve analysis of glucose metabolism, glycolysis or glucose uptake. Methods to classify a subject as metabolic responder is described in details in Example 1. Techniques to monitor glycolysis and glucose uptake is provided by T. TeSlaa and M.A. Teitell. 2014. Methods in Enzymology, Volume 542, pp. 92-114, incorporated herein by reference.

Glycolysis is the intracellular biochemical conversion of one molecule of glucose into two molecules of pyruvate with the concurrent generation of two molecules of ATP. Pyruvate is a metabolic intermediate with several potential fates including entrance into the tricarboxylic acid (TCA) cycle within mitochondria to produce NADH and FADH₂. Alternatively, pyruvate can be converted into lactate in the cytosol by lactate dehydrogenase with concurrent regeneration of NAD from NADH. An increased flux through glycolysis supports the proliferation of cancer cells by providing, for example, additional energy in the form of ATP as well as glucose-derived metabolic intermediates for nucleotide, lipid, and protein biosynthesis. Warburg (Oncologia. 1956;9(2):75-83) first observed that proliferating tumor cells augment aerobic glycolysis, the conversion of glucose to lactate in the presence of oxygen, in contrast to nonmalignant cells that mainly respire when oxygen is available. This mitochondrial bypass, called the Warburg effect, occurs in rapidly proliferating cells including cancer cells, activated lymphocytes, and pluripotent stem cells. The Warburg effect has been exploited for clinical diagnostic tests that use positron emission tomography (PET) scanning to identify increased cellular uptake of fluorinated glucose analogs such as 2-deoxy-2-(¹⁸F)-fluoro-D-glucose (also referred to herein as ¹⁸F-deoxyglucose, ¹⁸F-FDG, ¹⁸FDG, or FDG).

Thus, glycolysis represent a target for therapeutic and diagnostic methods. In the context of the current methods, the measurement of glucose uptake and lactate excretion by malignant cells may be useful to detect shifts in glucose catabolism and/or susceptibility to glucose metabolism inhibitors. Detecting such shifts is important for methods of treating GBM, methods of reducing the risk of ineffective therapy, methods for reducing the chances of tumor survival. For the purposes of this disclosure, ¹⁸F-deoxyglucose PET serves in certain embodiments as a rapid non-invasive functional biomarker to predict sensitivity to p53 activation. This non-invasive anlaysis could be particularly valuable for malignant brain tumors where pharmacokinetic/pharmacodynamics assessment is extremely difficult and impractical. In some cases, delayed imaging protocols (41) and parametric response maps (PRMs) with MRI fusion can be useful for quantifying the changes in tumore ¹⁸F-FDG uptake (42).

In certain aspects, the methods can relate to measuring glucose uptake and lactate production. For cells in culture, glycolytic flux can be quantified by measuring glucose uptake and lactate excretion. Glucose uptake into the cell is through glucose transporters (Glut1-Glut4), whereas lactate excretion is through monocarboxylate transporters (MCT1-MCT4) at the cell membrane.

### Extracellular Glucose and Lactate

Methods to detect glucose uptake and lactate excretion include, for example, extracellular glucose or lactate kit, extracellular bioanalyzer, ECAR measurement, [3H]-2-DG or [14C]-2-DG uptake ¹⁸FDG uptake or 2-NBDG uptake.

Commercially available kits and instruments are available to quantify glucose and lactate levels within cell culture media. Kit detection methods are usually colorimetric or fluorometric and are compatible with standard lab equipment such as spectrophotometers. BioProfile Analyzers (such as Nova Biomedical) or Biochemistry Analyzers (such as for example YSI Life Sciences) can measure levels of both glucose and lactate in cell culture media. GlucCell (Cesco BioProducts) can measure only glucose levels in cell culture media. While each commercial method has a different detection protocol, the collection of culture media for analysis is the same.

### Extracellular Acidification Rate

Glycolysis can also be determined through measurements of the extracellular acidification rate (ECAR) of the surrounding media, which is predominately from the excretion of lactic cid per unit time after its conversion from pyruvate. The Seahorse extracellular flux (XF) analyzer (Seahorse Bioscience) is a tool for measuring glycolysis and oxidative phosphorylation (through oxygen consumption) simultaneously in the same cells.

### Glucose Analog Uptake

Certain embodiments of the methods of the current disclosure include the use of glucose analogs. As would be familiar to a person skilled in the art, to determine the glucose uptake rate by cells, a labeled isoform of glucose can be added to the cell culture media and then measured within cells after a given period of time. Exemplary types of glucose analogs for these studies include but are not limited to radioactive glucose analogs, such as 2-deoxy-D[1,2-3H]-glucose, 2-deoxy-D[1-14C]-glucose, or 2-deoxy-2-(¹⁸F)-fluoro-D-glucose (¹⁸FDG), or fluorescent glucose analogs, such as 2-[N-(7-nitrobenz-2-oxa-1,3-diaxol-4-yl)amino]-2-deoxyglucose (2-NBDG). Measurements of radioactive glucose analog uptake require a scintillation counter, whereas 2-NBDG uptake is usually measured by flow cytometry or fluorescent microscopy. In some embodiments, the glucose uptake is measured by the uptake of radio-labelled glucose 2-deoxy-2-[fluorine-18]fluoro-D-glucose (¹⁸F-FDG). In further embodiments, detecting the ¹⁸F-FDG is by positron emission tomography (PET). In some embodiments, the biopsy is taken from a GBM tumor. A detailed description of an example of measuring ¹⁸F-FDG is provided in the examples below.

In certain aspects, the methods can relate to comparing glucose uptake of a biological sample such as a tumor sample with a control. Fold increases or decreases may be, be at least, or be at most 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100- or more, or any range derivable therein. Alternatively, differences in expression between a sample and a reference may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are with normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein. In some embodiments, the levels can be relative to a control.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered in some embodiments.

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by intimately bringing into association a compound of the present invention with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound, drug, or agent that is sufficient to elicit the desired therapeutic effect. The full therapeutic effect does not necessarily occur by administration of one dose, and may occur only after administration of a series of doses. Thus, a therapeutically effective amount may be administered in one or more administrations. It is generally understood that the effective amount of the compound will vary according to the weight, sex, age, and medical history of the subject, and the nature and extent of the condition being treated, such as cancer. Other factors which influence the effective amount may include, but are not limited to, the severity of the patient's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference). The skilled worker can readily determine the effective amount for a given situation by routine experimentation

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid , naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit EGFR may render them suitable as “therapeutic agents” in the methods and compositions of this disclosure. In some embodiements, the compounds or compositions of the disclosure inhibit EGFR or a variant thereof. In some embodiments, the EGFR or a variant thereof is ΔEGFR, an ex19 deletion, an EGFRvIII high-expression variant, or one or more EGFR amino acid mutants. In some embodiments, the one or more EGFR amino acid mutants is selected from L858R, C787S, C797X, L718Q, G724S, S768I, G719X, L792X, G796X, T263P, A289D, A289V, and G598V. In other embodiments, the one or more EGFR amino acid mutants is selected from C797S, G719A, L792H, L792F, L792Y, G796R and G796S.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release, slow release formulation, delayed release, or delayed and extended release, or administered using a device for such slow release,extended release, delayed release, or delayed and extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result in chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. In some embodiments, a straight chain or branched chain alkyl has 30 or fewer carbon atoms in its backbone (e.g., C₁₋₃₀ for straight chains, C₃₋₃₀ for branched chains), or 20 or fewer carbon atoms. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀ alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

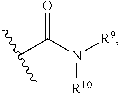

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

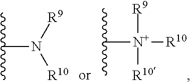

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

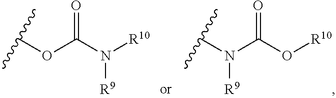

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

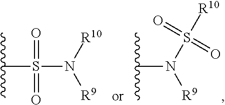

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

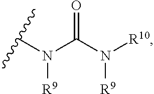

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula (I), Formula (Ia), or Formula (Ib). Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula (I), Formula (Ia), or Formula (Ib) are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula (I), Formula (Ia), or Formula (Ib) for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula (I), Formula (Ia), or Formula (Ib) or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of Formula (I), Formula (Ia), or Formula (lb)). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula (I), Formula (Ia), or Formula (Ib). The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### EXAMPLES

The disclosure now being generally described, it will be more readily understood by reference to the following examples which are included merely for purposes of illustration of certain aspects and embodiments of the present disclosure, and are not intended to limit the disclosure.

### Exemplary Design Rational for Certain Compounds of the JGK Series

Certain Compounds of the present disclosure were designed according to Scheme 1.

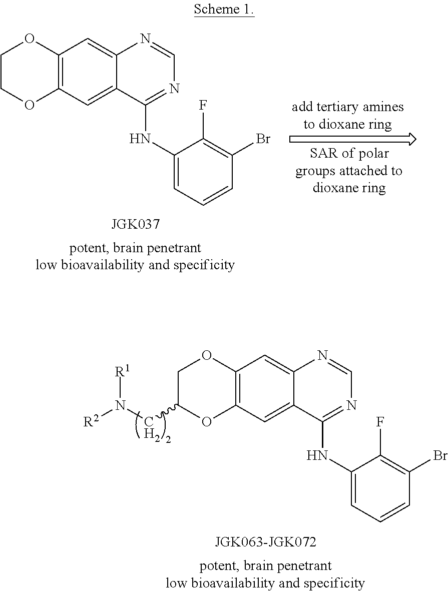

### Preparation of Exemplary Compounds of the JGK Series

Exemplary compounds of the present disclosure were prepared according to the following methods.

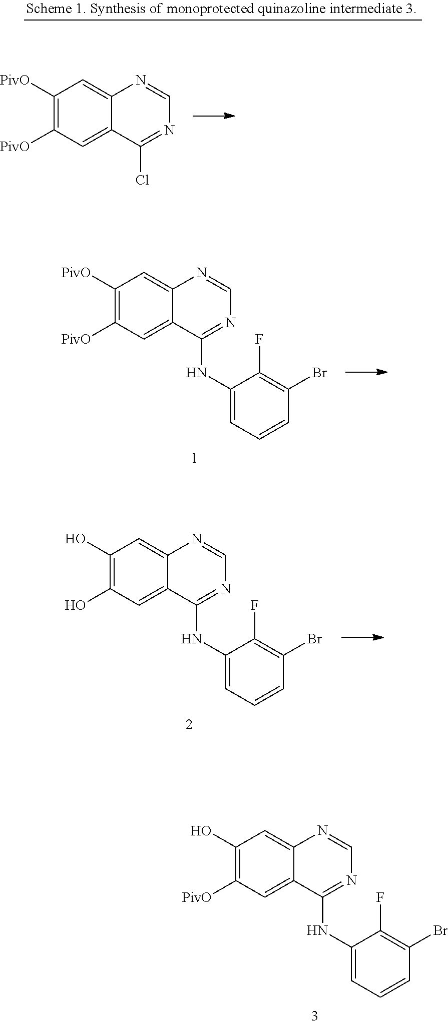

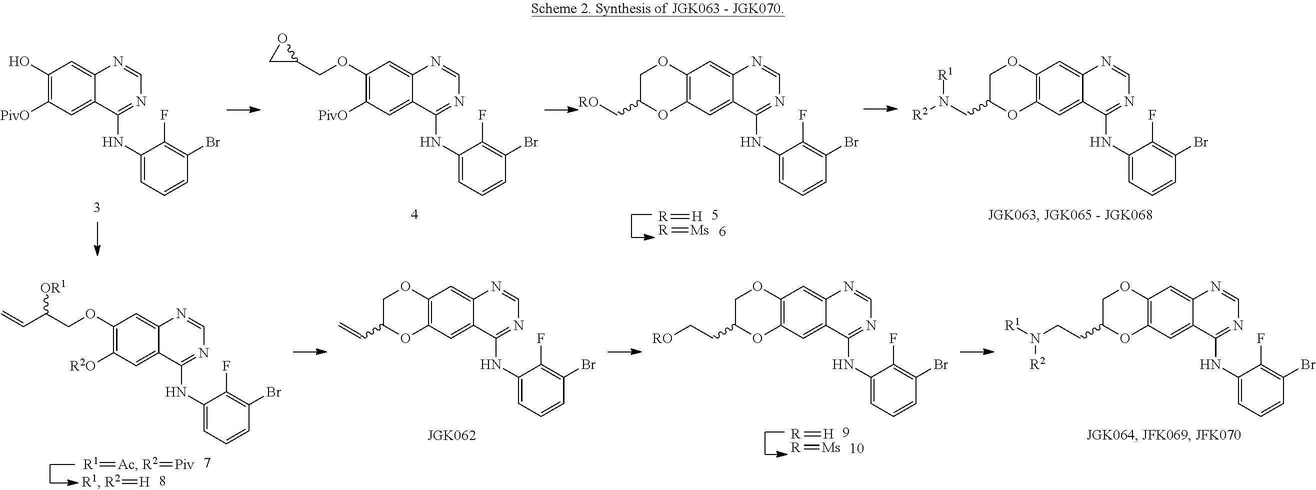

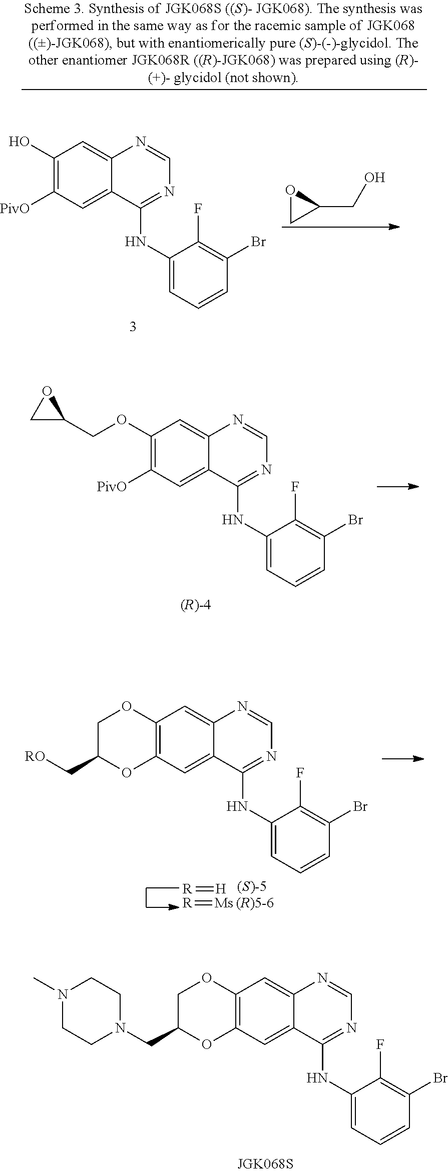

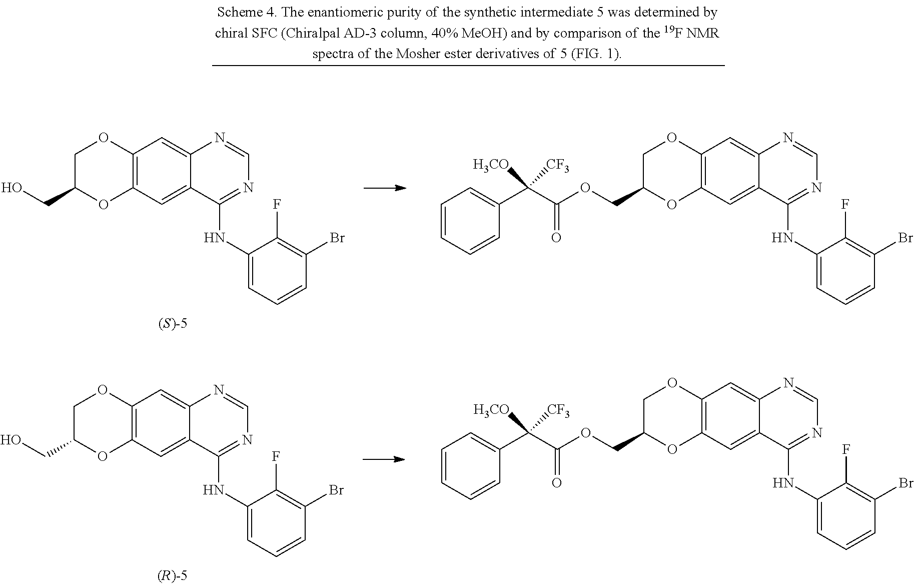 [SMILES: OC[C@H]1COc2cc3ncnc(Nc4cccc(Br)c4F)c3cc2O1 ; CO[C@@](C(=O)OC[C@H]1COc2cc3ncnc(Nc4cccc(Br)c4F)c3cc2O1)(c1ccccc1)C(F)(F)F ; OC[C@@H]1COc2cc3ncnc(Nc4cccc(Br)c4F)c3cc2O1 ; CO[C@@](C(=O)OC[C@H]1COc2cc3ncnc(Nc4cccc(Br)c4F)c3cc2O1)(c1ccccc1)C(F)(F)F]

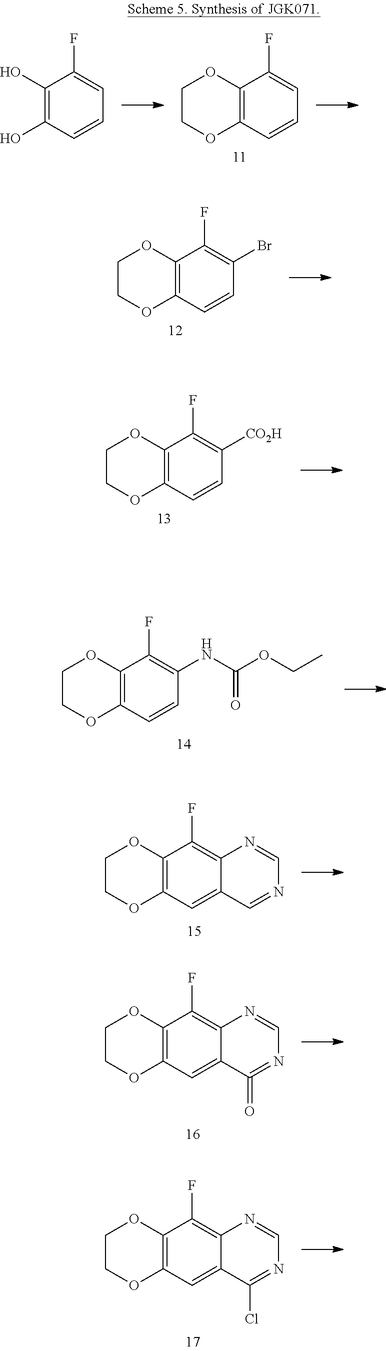

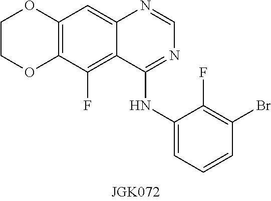 [SMILES: Fc1c(Br)cccc1Nc1ncnc2cc3c(c(F)c12)OCCO3]

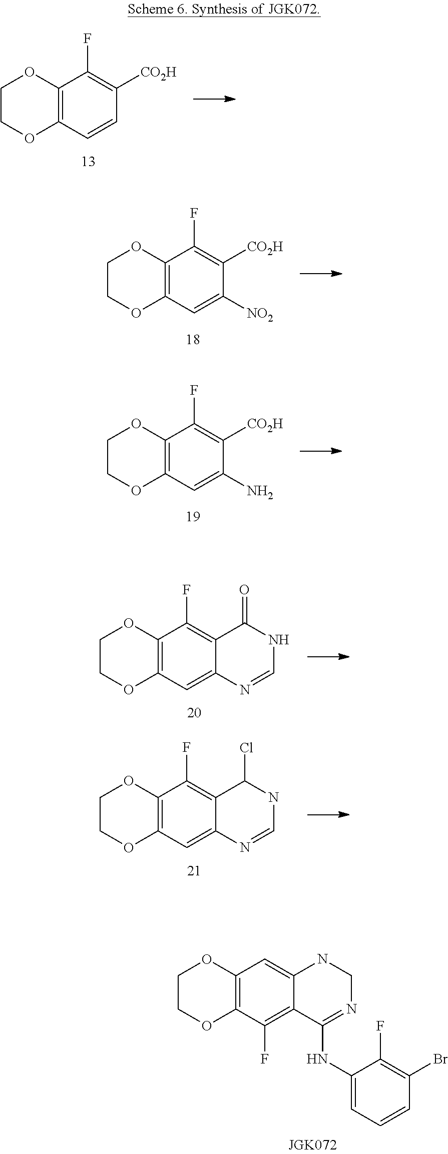 [SMILES: O=C(O)c1ccc2c(c1F)OCCO2 ; O=C(O)c1c([N+](=O)[O-])cc2c(c1F)OCCO2 ; Nc1cc2c(c(F)c1C(=O)O)OCCO2 ; O=c1[nH]cnc2cc3c(c(F)c12)OCCO3 ; Fc1c2c(cc3c1C(Cl)NC=N3)OCCO2 ; Fc1c(Br)cccc1NC1=NCNc2cc3c(c(F)c21)OCCO3]

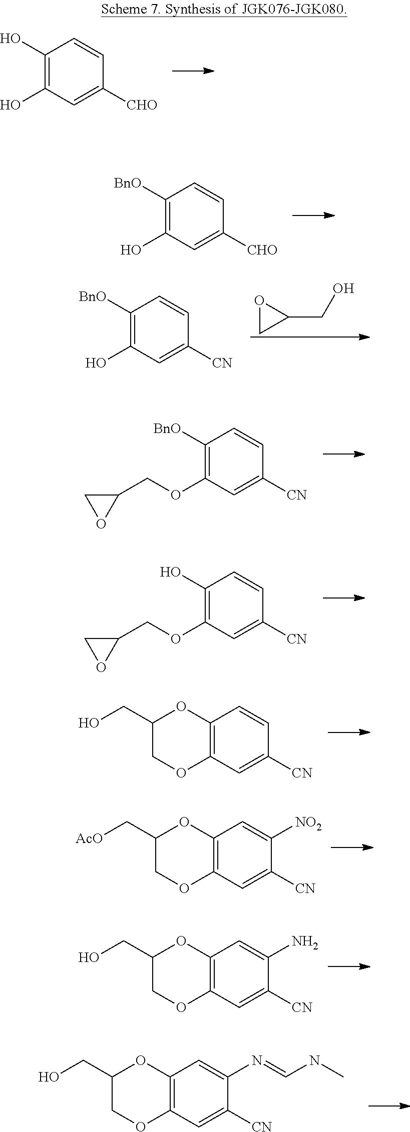

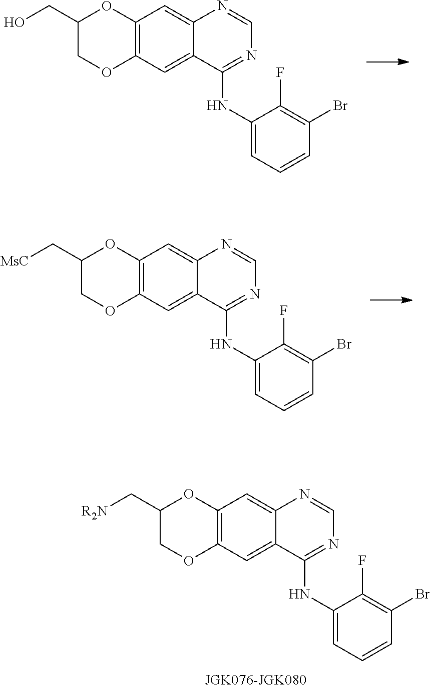

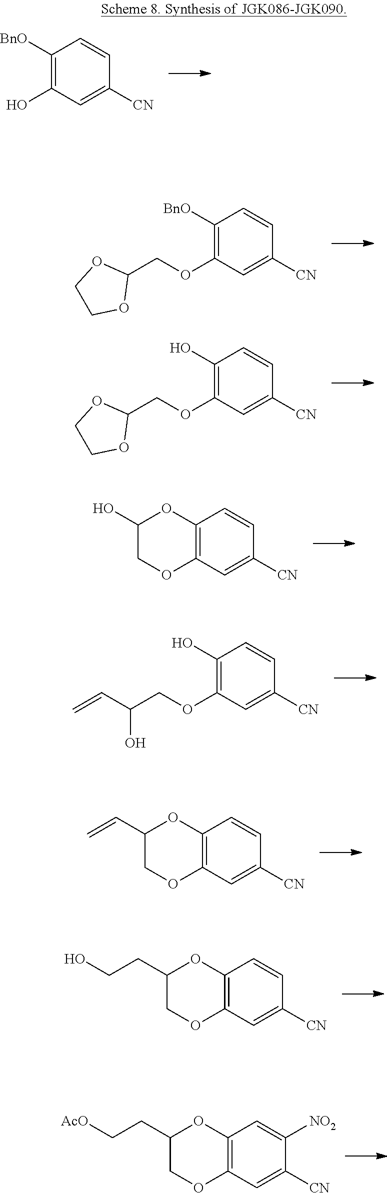

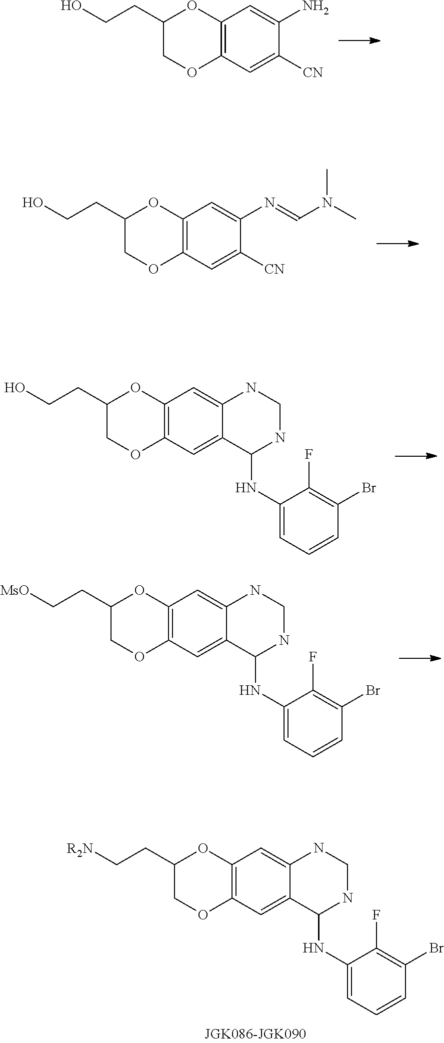

### General Chemistry Information

All chemicals, reagents, and solvents were purchased from commercial sources when available and were used as received. When necessary, reagents and solvents were purified and dried by standard methods. Air- and moisture-sensitive reactions were carried out under an inert atmosphere of argon in oven-dried glassware. Microwave-irradiated reactions were carried out in a single mode reactor CEM Discover microwave synthesizer. Room temperature (RT) reactions were carried out at ambient temperature (approximately 23° C.). All reactions were monitored by thin layer chromatography (TLC) on precoated Merck 60 F₂₅₄ silica gel plates with spots visualized by UV light (λ=254, 365 nm) or by using an alkaline KMnO₄ solution. Flash column chromatography (FC) was carried out on SiO₂ 60 (particle size 0.040-0.063 mm, 230-400 mesh). Preparative thin-layer chromatography (PTLC) was carried out with Merck 60 F₂₅₄ silica gel plates (20×20 cm, 210-270 mm) or Analtech Silica Gel GF TLC plates (20×20 cm, 1000 mm). Concentration under reduced pressure (in vacuo) was performed by rotary evaporation at 23-50° C. Purified compounds were further dried under high vacuum or in a desiccator. Yields correspond to purified compounds, and were not further optimized. Proton nuclear magnetic resonance (¹H NMR) spectra were recorded on Bruker spectrometers (operating at 300, 400, or 500 MHz). Carbon NMR (¹³C NMR) spectra were recorded on Bruker spectrometers (either at 400 or 500 MHz). NMR chemical shifts (8 ppm) were referenced to the residual solvent signals. ¹H NMR data are reported as follows: chemical shift in ppm; multiplicity (s=singlet, d=doublet, t=triplet, q=quartet, quint=quintet, m=multiplet/complex pattern, td=triplet of doublets, ddd=doublet of doublet of doublets, br=broad signal); coupling constants (J) in Hz, integration. Data for ¹³C NMR spectra are reported in terms of chemical shift, and if applicable coupling constants. High resolution mass (HRMS) spectra were recorded on a Thermo Fisher Scientific Exactive Plus with IonSense ID-CUBE DART source mass spectrometer, or on a Waters LCT Premier mass spectrometer with ACQUITY UPLC with autosampler.

General Procedures (GP). GP-1: Nucleophilic Substitution of Quinazolinyl Mesylates with Secondary Amines. A mixture of quinazolinyl mesylate (1 equiv) in DMF (0.05 M) was treated with the secondary amine (5 equiv) and triethylamine (2 equiv), and the mixture was stirred at 85° C. for 24 h. The mixture was cooled to 23° C., and evaporated. The residue was dissolved in EtOAc (20 mL), washed with 10 mm NaOH (4×5 mL), brine (5 mL), dried (Na₂SO₄), filtered, and evaporated. Purification by FC or PTLC afforded the desired products typically as off-white, friable foams.

GP-2: Nucleophilic Aromatic Substitution of 4-Chloroquinazoline with Anilines. A mixture of 4-chloroquinazoline (1 equiv) in acetonitrile (0.1 M) was treated with aniline (2 equiv), and with a 4 M solution of HCl in dioxane (1 equiv). The mixture was heated at 80° C. under microwave irradiation for 30 min. The mixture was either concentrated under reduced pressure, or the precipitated 4-anilinoquinazoline hydrochloride salt was isolated by filtration (washings with Et₂O). The residue was suspended in sat. aq. NaHCO₃, and extracted with CH₂Cl₂ (3×). The combined organic extracts were washed with water, brine, dried (Na₂SO₄), filtered, and concentrated. Purification by FC (elution with a gradient of CH₂Cl₂/EtOAc or hexanes/EtOAc) afforded the desired products typically as white to off-white, or pale-yellow solids.

### 4-(3-Bromo-2-fluoroanilino)quinazoline-6,7-diyl bis(2,2-dimethylpropanoate) (1)

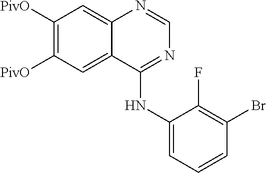

A mixture of 4-chloroquinazoline-6,7-diyl bis(2,2-dimethylpropanoate)¹ (41.08 g, 113 mmol) in iPrOH (450 mL) was treated with 3-bromo-2-fluoroaniline (17.05 mL, 152 mmol) and stirred at 80° C. for 3.5 h. The mixture was cooled to 23° C. and evaporated. The residue was several times resuspended in hexanes (50 mL) and concentrated, and then dried under HV. The residue was recrystallized from EtOH to give a yellow solid, which was suspended in sat. aq. NaHCO₃ (1 L), and extracted with DCM (3×550 mL). The combined organics were washed with water (400 mL), brine (400 mL), dried (MgSO₄), filtered, and evaporated to afford the title compound 1 (35.057 g, 60%) as a yellow friable foam.

¹H NMR (500 MHz, CDCl₃): δ=8.76 (s, 1H), 8.46 (t, J=7.5 Hz, 1H), 7.72 (s, 1H), 7.68 (s, 1H), 7.56 (br, 1H), 7.32 (ddd, J=8.0, 6.4, 1.5 Hz, 1H), 7.11 (td, J=8.2, 1.5 Hz, 1H), 1.40 (s, 9H), 1.39 ppm (s, 9H). ¹³C NMR (126 MHz, CDCl₃): δ=176.13, 175.55, 156.71, 154.96, 150.69 (d, J_{CF}=243.7 Hz), 148.75, 147.83, 142.45, 128.27, 127.86 (d, J_{CF}=10.8 Hz), 125.29 (d, J_{CF}=4.7 Hz), 122.70, 122.51, 114.43, 113.21, 108.84 (d, J_{CF}=19.4 Hz), 39.54, 39.51, 27.40, 27.32 ppm. HRMS (DART): m/z [M+H]⁺ calcd for C₂₄H₂₆BrFN₃O₄⁺, 518.1085; found, 518.1072.

### 4-(3-Bromo-2-fluoroanilino)quinazoline-6,7-diol (2)

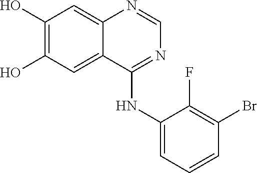 [SMILES: Oc1cc2ncnc(Nc3cccc(Br)c3F)c2cc1O]

A stirred slurry of 1 (34.988 g, 67.5 mmol) was treated at 0° C. with 7 M solution of NH₃ in MeOH (241 mL, 1.69 mol). The mixture was stirred at 0° C. for 15 min, and then at 23° C. for 4.5 h. The mixture was evaporated, and the residue suspended in water (400 mL), stirred overnight, and filtered. The residue was washed with water (500 mL), acetonitrile (100 mL), DCM (4×150 mL), Et₂O (2×150 mL), and dried in a desiccator to afford the title compound 2 (23.68 g, quant.) as a pale-yellow powder.

¹H NMR (500 MHz, DMSO-d₆): δ=8.18 (s, 1H), 7.59-7.47 (m, 2H), 7.51 (s, 1H), 7.16 (t, J=8.0 Hz, 1H), 6.87 ppm (s, 1H). ¹³C NMR (126 MHz, DMSO-d₆): δ=156.43, 156.12, 153.06 (d, J_{CF}=246.7 Hz), 151.34, 148.39, 146.80, 129.23, 129.01, 127.12, 125.23 (d, J_{CF}=4.3 Hz), 108.47, 108.32, 107.09, 103.04 ppm. HRMS (DART): m/z [M+H]⁺ calcd for C₁₄H₁₀BrFN₃O₂⁺, 349.9935; found, 349.9923.

### 4-(3-Bromo-2-fluoroanilino)-7-hydroxyquinazolin-6-yl 2,2-dimethylpropanoate (3)

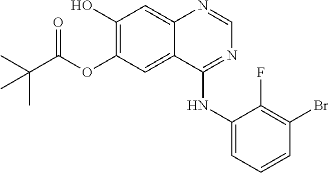 [SMILES: CC(C)(C)C(=O)Oc1cc2c(Nc3cccc(Br)c3F)ncnc2cc1O]

A stirred suspension of 2 (3500 mg, 10.0 mmol) in DMF (52.6 mL) was treated with Et₃N (5.57 mL, 40.0 mmol), cooled to −40° C., and treated dropwise with Piv₂O (3.14 mL, 15.5 mmol). The mixture was stirred at −40° C. for 1 h, after which the cooling bath was removed, and stirring was continued for 2.5 h. The reaction mixture was diluted with DCM (500 mL), washed with 10% citric acid (2×50 mL), dried (Na₂SO₄), filtered, and evaporated. FC (DCM/EtOAc 1:1→0:1) afforded a solid, which was redissolved in EtOAc (750 mL), and washed with half-sat. aq. NH₄Cl (4×75 mL), dried (Na₂SO₄), filtered, and evaporated to afford the title compound 3 (2.844 g, 66%) as a beige-yellow solid.

¹H NMR (500 MHz, DMSO-d₆): δ=11.00 (br, 1H), 9.70 (s, 1H), 8.39 (s, 1H), 8.14 (s, 1H), 7.59 (ddd, J=8.0, 6.2, 1.6 Hz, 1H), 7.53 (ddd, J=8.3, 7.1, 1.6 Hz, 1H), 7.21 (td, J=8.1, 1.2 Hz, 1H), 7.17 (s, 1H), 1.36 ppm (s, 9H). ¹³C NMR (126 MHz, DMSO-d₆): δ=175.93, 157.68, 154.61, 154.53, 153.34 (d, J_{CF}=247.3 Hz), 149.80, 139.65, 130.14, 127.92 (d, J_{CF}=12.9 Hz), 127.62, 125.47 (d, J_{CF}=4.4 Hz), 116.36, 111.00, 108.55 (d, J=20.0 Hz), 107.77, 38.64, 26.93 ppm. HRMS (DART): m/z [M+H]⁺ calcd for C₁₉H₁₈BrFN₃O₃⁺, 434.0510; found, 434.0489.

### (±)-4-(3-Bromo-2-fluoroanilino)-7-[(oxiran-2-yl)methoxy]quinazolin-6-yl 2,2-dimethylpropanoate ((±)-4)

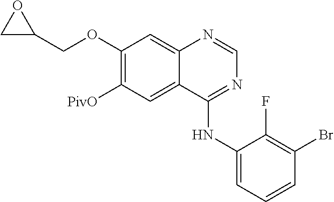

A mixture of 3 (1350 mg, 3.11 mmol) and PPh₃ (2038 mg, 7.77 mmol) in THF (21 mL) was treated with glycidol (495 μL, 7.46 mmol), cooled to 0° C., and treated with DIAD (1.47 mL, 7.46 mmol) during 10 min The mixture was stirred at 23° C. for 2.5 h, and concentrated. FC (DCM/EtOAc 9:1→4:6) afforded the title compound (±)-4 (848 mg, 56%) as an off-white solid.

¹H NMR (500 MHz, CDCl₃): δ=8.73 (s, 1H), 8.54 (ddd, J=8.6, 7.3, 1.6 Hz, 1H), 7.54 (s, 1H), 7.45 (br, 1H), 7.30 (ddd, J=8.2, 6.4, 1.5 Hz, 1H), 7.28 (s, 1H), 7.11 (td, J=8.2, 1.6 Hz, 1H), 4.34 (dd, J=10.8, 3.0 Hz, 1H), 3.99 (dd, J=10.8, 6.2 Hz, 1H), 3.35 (ddt, J=6.2, 4.1, 2.8 Hz, 1H), 2.92 (dd, J=4.8, 4.1 Hz, 1H), 2.74 (dd, J=4.8, 2.6 Hz, 1H), 1.45 ppm (s, 9H). ¹³C NMR (126 MHz, CDCl₃): δ=176.87, 156.46, 155.10, 154.93, 150.41 (d, J_{CF}=243.3 Hz), 150.27, 140.99, 128.25 (d, J_{CF}=10.5 Hz), 127.75, 125.28 (d, J_{CF}=4.7 Hz), 122.22, 114.02, 109.72, 109.49, 108.74 (d, J_{CF}=19.1 Hz), 70.05, 49.55, 44.56, 39.45, 27.38 ppm. HRMS (DART): m/z [M+H]⁺ calcd for C₂₂H₂₂BrFN₃O₄⁺, 490.0772; found, 490.0764.

### (±)-N-(3-Bromo-2-fluorophenyl)-7-ethenyl-7,8-dihydro[1,4]dioxino[2,3-g]quinazolin-4-amine ((±)-JGK062)

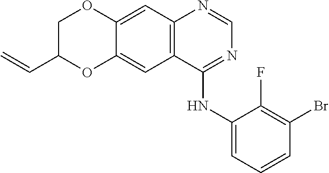 [SMILES: C=CC1COc2cc3ncnc(Nc4cccc(Br)c4F)c3cc2O1]

A solution of PPh₃ (832 mg, 3.17 mmol) and DIAD (624 μL, 3.17 mmol) in THF (23 mL) was stirred at 0° C. for 15 min, and then added dropwise to a solution of (±)-8 (1149 mg, 2.73 mmol) in THF (27 mL) during 10 min at 0° C. The mixture was stirred at 0° for 2 h, and evaporated. FC (hexanes/EtOAc 9:1→4:6) followed by another FC (DCM/EtOAc 1:0→6:4) afforded the title compound (±)-JGK062 (1115 mg, quant.) as an off-white friable foam.

¹H NMR (500 MHz, CDCl₃): δ=8.68 (s, 1H), 8.65 (ddd, J=8.2, 7.3, 1.5 Hz, 1H), 7.40 (s, 1H), 7.37 (br, 1H), 7.35 (s, 1H), 7.27 (ddd, J=8.0, 6.4, 1.5 Hz, 1H), 7.10 (td, J=8.2, 1.6 Hz, 1H), 5.95 (ddd, J=17.3, 10.7, 5.8 Hz, 1H), 5.60 (dt, J=17.3, 1.2 Hz, 1H), 5.48 (dt, J=10.7, 1.1 Hz, 1H), 4.82-4.74 (m, 1H), 4.42 (dd, J=11.5, 2.5 Hz, 1H), 4.09 ppm (dd, J=11.6, 8.1 Hz, 1H). ¹³C NMR (126 MHz, CDCl₃): δ=155.90, 153.38, 150.14 (d, J=242.4 Hz), 149.12, 146.70, 144.12, 131.48, 128.64 (d, J=10.3 Hz), 127.24, 125.30 (d, J=4.7 Hz), 121.76, 120.43, 114.29, 110.69, 108.58 (d, J=19.3 Hz), 106.06, 74.03, 67.84 ppm. HRMS (DART): m/z [M+H]⁺ calcd for C₁₈H₁₄BrFN₃O₂⁺, 402.0248; found, 402.0233.

### (±)-[4-(3-Bromo-2-fluoroanilino)-7,8-dihydro[1,4]dioxino[2,3-g]quinazolin-7-yl]methanol ((±)-5)

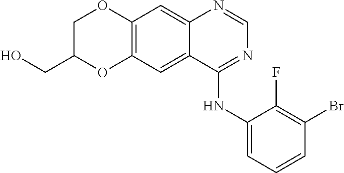 [SMILES: OCC1COc2cc3ncnc(Nc4cccc(Br)c4F)c3cc2O1]

A mixture of (±)-4 (842 mg, 1.72 mmol) in MeOH (31 mL) was treated with K₂CO₃ (482 mg, 3.49 mmol), stirred at 23° C. for 10.5 h, and concentrated. The residue was suspended in half-sat. aq. NH₄Cl (130 mL), and extracted with EtOAc (3×20 mL). The combined organics were washed with water (20 mL), brine (20 mL), dried (Na₂SO₄), filtered, and concentrated to afford the title compound (±)-5 (720 mg, quant.) as a yellow solid.

¹H NMR (500 MHz, DMSO-d₆): δ=9.59 (s, 1H), 8.34 (s, 1H), 7.95 (s, 1H), 7.59 (ddd, J=8.0, 6.2, 1.6 Hz, 1H), 7.55 (ddd, J=8.4, 7.0, 1.6 Hz, 1H), 7.24-7.18 (m, 1H), 7.21 (s, 1H), 5.16 (t, J=5.6 Hz, 1H), 4.49 (dd, J=11.5, 2.4 Hz, 1H), 4.34 (dtd, J=7.6, 5.2, 2.3 Hz, 1H), 4.21 (dd, J=11.5, 7.4 Hz, 1H), 3.76-3.64 ppm (m, 2H). ¹³C NMR (126 MHz, DMSO-d₆): δ=157.20, 153.35 (d, J_{CF}=247.5 Hz), 153.10, 148.88, 145.95, 143.39, 130.11, 128.05 (d, J_{CF}=13.0 Hz), 127.73, 125.44 (d, J_{CF}=4.4 Hz), 112.33, 109.79, 108.56 (d, J_{CF}=20.0 Hz), 108.37, 73.78, 65.50, 59.78 ppm. HRMS (DART): m/z [M+H]⁺ calcd for C₁₇H₁₄BrFN₃O₃⁺, 406.0197; found, 406.0185.

### (±)-[4-(3-Bromo-2-fluoroanilino)-7,8-dihydro[1,4]dioxino[2,3-g]quinazolin-7-yl]methyl methanesulfonate ((±)-6)

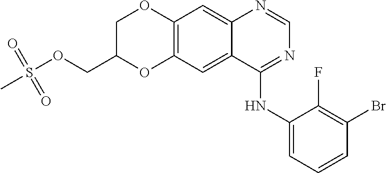 [SMILES: CS(=O)(=O)OCC1COc2cc3ncnc(Nc4cccc(Br)c4F)c3cc2O1]

A solution of (±)-5 (688 mg, 1.69 mmol) in THF (14 mL) was treated with Et₃N (357 μL, 2.56 mmol), cooled to 0° C., and treated dropwise with MsCl (174 μL, 2.24 mmol). The mixture was stirred at 23° C. for 16 h, cooled to 0° C., treated with sat. aq. NaHCO₃ (120 mL), and extracted with DCM (3×120 mL). The combined organics were washed with water (100 mL), brine (100 mL), dried (Na₂SO₄), filtered and evaporated. FC (DCM/EtOAc 8:2→3:7) afforded the title compound (±)-6 (496 mg, 61%) as an off-white solid.

¹H NMR (500 MHz, CDCl₃): δ=8.69 (s, 1H), 8.60 (ddd, J=8.5, 7.2, 1.4 Hz, 1H), 7.43 (s, 1H), 7.39 (br, 1H), 7.37 (s, 1H), 7.29 (ddd, J=8.1, 6.5, 1.5 Hz, 1H), 7.11 (td, J=8.2, 1.5 Hz, 1H), 4.63 (dtd, J=7.2, 4.9, 2.5 Hz, 1H), 4.52 (dd, J=4.9, 0.9 Hz, 2H), 4.49 (dd, J=11.8, 2.5 Hz, 1H), 4.29 (dd, J=11.8, 7.1 Hz, 1H), 3.13 ppm (s, 3H). ¹³C NMR (126 MHz, CDCl₃): δ=156.02, 153.66, 150.28 (d, J_{CF}=242.9 Hz), 148.65, 146.80, 143.09, 128.43 (d, J_{CF}=10.4 Hz), 127.54, 125.32 (d, J_{CF}=4.7 Hz), 122.01, 114.77, 110.90, 108.66 (d, J_{CF}=19.4 Hz), 106.44, 71.10, 66.46, 64.77, 38.02 ppm. HRMS (DART): m/z [M+H]⁺ calcd for C₁₈H₁₅BrFN₃O₅S⁺, 483.9973; found, 483.9950.

### (±)-4-(3-Bromo-2-fluoroandino)-7-{[2-(acetoxy)but-3-en-1-yl]oxy}quinazolin-6-yl 2,2-dimethylpropanoate ((±)-7)

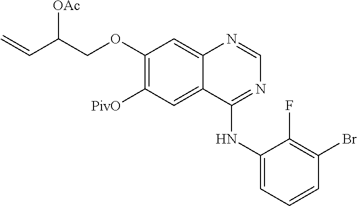

A mixture of 3 (2639 mg, 6.08 mmol) and PPh₃ (3986 mg, 15.2 mmol) in THF (41 mL) was treated with racemic 1-hydroxybut-3-en-2-yl acetate² (1.7 mL, 13.7 mmol), cooled to 0° C., and treated dropwise with DIAD (2.7 mL, 13.7 mmol). The mixture was stirred at 23° C. for 3 h, and concentrated. FC (DCM/EtOAc 1:0→6:4) afforded the crude (±)-7 (5.508 g, estimated yield 60%) as an off-white solid, which was contaminated with remaining Ph₃PO. The material was used in the next step without any further purification.

¹H NMR (400 MHz, CDCl₃): δ=8.74 (s, 1H), 8.53 (t, J=7.9 Hz, 1H), 7.53 (s, 1H), 7.45 (br, 1H), 7.33 (s, 1H), 7.30 (t, J=7.7 Hz, 1H), 7.11 (t, J=8.0 Hz, 1H), 5.90 (ddd, J=17.0, 10.6, 6.2 Hz, 1H), 5.65 (q, J=6.0 Hz, 1H), 5.49-5.29 (m, 2H), 4.31-4.08 (m, 2H), 2.11 (s, 3H), 1.41 ppm (s, 9H). ¹³C NMR (126 MHz, CDCl₃): δ=176.51, 170.08, 156.49, 155.24, 154.88, 150.46 (d, J_{CF}=243.2 Hz), 150.17, 140.90, 132.16, 128.18 (d, J_{CF}=11.0 Hz), 127.86, 125.31 (d, J_{CF}=4.8 Hz), 122.27, 119.64, 114.00, 109.56, 109.39, 108.76 (d, J_{CF}=19.4 Hz), 72.18, 69.81, 39.34, 27.33, 21.19 ppm. HRMS (DART): m/z [M+H]⁺ calcd for C₂₅H₂₆BrFN₃O₅⁺, 546.1034; found, 546.1018.

### (±)-4-(3-Bromo-2-fluoroanilino)-7-[(2-hydroxybut-3-en-1-yl)oxy]quinazolin-6-ol ((±)-8)

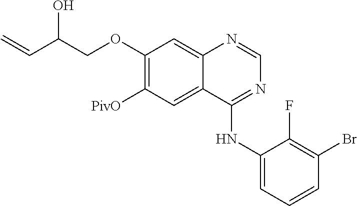

A mixture of crude (±)-7 (5508 mg, contaminated with remaining Ph₃PO from the last step) in MeOH (61 mL) was treated with K₂CO₃ (4198 mg, 30.4 mmol), stirred at 23° C. for 1 h, and concentrated. The residue was suspended in half-sat. aq. NH₄Cl (1 L), and extracted with EtOAc (3×600 mL). The combined organics were dried (Na₂SO₄), filtered, and evaporated. FC (DCM/EtOAc 1:1→0:1) afforded the title compound (±)-8 (1154 mg, 45% over two steps) as an off-white solid.

¹H NMR (500 MHz, DMSO-d₆): δ=9.46 (s, 1H), 9.40 (br, 1H), 8.33 (s, 1H), 7.71 (s, 1H), 7.59-7.52 (m, 2H), 7.203 (s), 7.197 (td, J=8.1, 1.1 Hz, 1H), 6.01 (ddd, J=17.4, 10.7, 4.9 Hz, 1H), 5.42 (dt, J=17.3, 1.9 Hz, 1H), 5.36 (br, 1H), 5.20 (dt, J=10.6, 1.8 Hz, 1H), 4.49 (br, 1H), 4.20 (dd, J=9.8, 3.8 Hz, 1H), 3.95 ppm (dd, J=9.8, 7.5 Hz, 1H). ¹³C NMR (126 MHz, DMSO-d₆): δ=156.77, 153.30 (d, J_{CF}=244.9 Hz), 152.77, 152.31, 146.66, 146.11, 137.61, 129.75, 128.46 (d, J_{CF}=13.0 Hz), 127.49, 125.38 (d, J_{CF}=4.3 Hz), 115.58, 109.42, 108.50 (d, J_{CF}=19.8 Hz), 107.68, 105.14, 72.56, 69.26 ppm. HRMS (DART): m/z [M+H]⁺ calcd for C₁₈H₁₆BrFN₃O₃⁺, 420.0354; found, 420.0340.

### (±)-2-[4-(3-Bromo-2-fluoroanilino)-7,8-dihydro[1,4]dioxino[2,3-g]quinazolin-7-yl]ethan-1-ol ((±)-9)

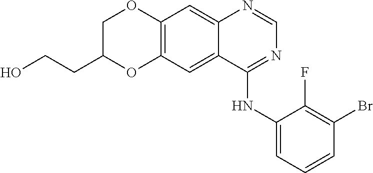 [SMILES: OCCC1COc2cc3ncnc(Nc4cccc(Br)c4F)c3cc2O1]

A mixture of (±)-JGK062 (480 mg, 1.19 mmol) in THF (4.8 mL) was treated with a 0.5 M solution of 9-BBN in THF (4.8 mL, 2.39 mmol), and the mixture was stirred at 68° C. for 16 h. The mixture was cooled to 0° C., diluted with THF (2.4 mL), and treated with 3 N NaOH (3 mL, 8.95 mmol), and 30% H₂O₂ (474 μL, 8.95 mmol), and stirred at 23° C. for 6 h. The mixture was concentrated to about half of the original volume of THF, diluted with water (100 mL) and brine (40 mL), and extracted with EtOAc (3×100 mL). The combined organics were washed with water (70 mL), brine (70 mL), dried (Na₂SO₄), filtered, and evaporated to afford the title compound (±)-9 (912 mg) as a yellow foam, which was directly used in the next step without further purification.

¹H NMR (500 MHz, CDCl₃): δ=8.66 (s, 1H), 8.62 (ddd, J=8.8, 7.4, 1.6 Hz, 1H), 7.35 (s, 1H), 7.33 (br, 1H), 7.2 (ddd, J=8.0, 6.5, 1.6 Hz, 1H), 7.16 (s, 1H), 7.09 (td, J=8.2, 1.6 Hz, 1H), 4.50 (dtd, J=8.4, 6.4, 2.3 Hz, 1H), 4.43 (dd, J=11.5, 2.3 Hz, 1H), 4.09 (dd, J=11.5, 8.2 Hz, 1H), 4.01-3.91 (m, 2H), 1.95 ppm (td, J=6.5, 5.3 Hz, 2H). ¹³C NMR (126 MHz, CDCl₃): δ=155.84, 153.28, 150.08 (d, J_{CF}=242.6 Hz), 149.42, 146.47, 144.20, 128.51 (d, J_{CF}=10.2 Hz), 127.31, 125.30 (d, J_{CF}=4.7 Hz), 121.69, 113.95, 110.50, 108.58 (d, J_{CF}=19.2 Hz), 105.83, 71.33, 68.49, 58.23, 33.61 ppm. HRMS (ESI): m/z [M+H]⁺ calcd for C₁₈H₁₆BrFN₃O₃⁺, 420.0354; found, 420.0370.

### (±)-2-[4-(3-Bromo-2-fluoroanilino)-7,8-dihydro[1,4]dioxino[2,3-g]quinazolin-7-yl]ethyl methanesulfonate ((±)-10)

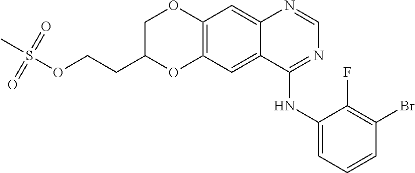 [SMILES: CS(=O)(=O)OCCC1COc2cc3ncnc(Nc4cccc(Br)c4F)c3cc2O1]

A solution of crude (±)-9 (912 mg) in THF (11.9 mL) was treated with Et₃N (931 mL, 6.68 mmol), cooled to 0° C., and treated dropwise with MsCl (462 μL, 5.97 mmol). The mixture was stirred at 0° C. for 15 min, and then at 23° C. for 21 h. The mixture was cooled to 0° C., treated dropwise with sat. aq. NaHCO₃ (120 mL), and extracted with DCM (3×120 mL). The combined organics were washed with water (100 mL), brine (100 mL), dried (Na₂SO₄), filtered, and evaporated. FC (DCM/EtOAc 9:1→4:6) afforded the title compound (±)-10 (112 mg, 19% over two steps) as an off-white, friable foam.

¹H NMR (500 MHz, CDCl₃): δ=8.68 (s, 1H), 8.60 (ddd, J=8.6, 7.3, 1.5 Hz, 1H), 7.44 (br, 1H), 7.42 (s, 1H), 7.35 (s, 1H), 7.29 (ddd, J=8.1, 6.5, 1.6 Hz, 1H), 7.11 (td, J=8.2, 1.5 Hz, 1H), 4.60-4.48 (m, 3H), 4.44 (dd, J=11.6, 2.4 Hz, 1H), 4.12 (dd, J=11.6, 7.6 Hz, 1H), 3.08 (s, 3H), 2.24-2.10 ppm (m, 2H). ¹³C NMR (126 MHz, CDCl₃): δ=156.03, 153.39, 150.31 (d, J_{CF}=242.9 Hz), 149.11, 146.54, 143.60, 128.47 (d, J_{CF}=10.5 Hz), 127.52, 125.32 (d, J_{CF}=4.6 Hz), 122.02, 114.30, 110.68, 108.66 (d, J_{CF}=19.2 Hz), 106.32, 69.78, 67.82, 65.05, 37.75, 30.90 ppm. HRMS (ESI): m/z [M+H]⁺ calcd for C₁₉H₁₈BrFN₃O₅S⁺, 498.0129; found, 498.0144.

### (±)-N-(3-Bromo-2-fluorophenyl)-7-[(morpholin-4-yl)methyl]-7,8-dihydro[1,4]dioxino[2,3-g]quinazolin-4-amine ((±)-JGK063)

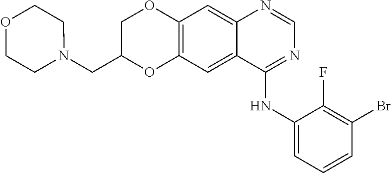 [SMILES: Fc1c(Br)cccc1Nc1ncnc2cc3c(cc12)OC(CN1CCOCC1)CO3]

Following general procedure GP-1, compound (±)-JGK063 was prepared from (±)-6 (20 mg, 0.04 mmol) and morpholine (18 μL, 0.21 mmol) in DMF (826 μL). PTLC (DCM/EtOAc 1:9) afforded (±)-JGK063 (15 mg, 76%) as an off-white, friable foam.

¹H NMR (500 MHz, CDCl₃): δ=8.67 (s, 1H), 8.63 (ddd, J=8.6, 7.3, 1.5 Hz, 1H), 7.38 (s, 1H), 7.37 (br, 1H), 7.31 (s, 1H), 7.27 (ddd, J=8.0, 6.3, 1.5 Hz, 1H), 7.10 (td, J=8.2, 1.5 Hz, 1H), 4.50-4.41 (m, 2H), 4.21-4.12 (m, 1H), 3.75 (t, J=4.7 Hz, 4H), 2.77 (dd, J=13.4, 5.9 Hz, 1H), 2.69-2.54 ppm (m, 5H). ¹³C NMR (126 MHz, CDCl₃): δ=155.89, 153.36, 150.15 (d, J_{CF}=242.5 Hz), 149.35, 146.66, 144.02, 128.60 (d, J_{CF}=10.4 Hz), 127.27, 125.30 (d, J_{CF}=4.6 Hz), 121.80, 114.29, 110.63, 108.58 (d, J_{CF}=19.5 Hz), 106.06, 71.61, 67.18, 67.01, 58.94, 54.56 ppm. HRMS (ESI): m/z [M−H]⁻ calcd for C₂₁H₁₉BrFN₄O₃⁻, 473.0630; found, 473.0630.

### (±)-N-(3-Bromo-2-fluorophenyl)-7-[2-(morpholin-4-yl)ethyl]-7,8-dihydro[1,4]dioxino[2,3-g]quinazolin-4-amine ((±)-JGK064)

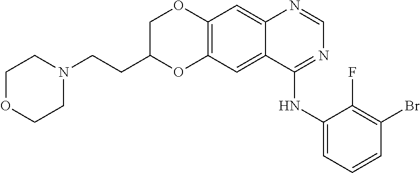 [SMILES: Fc1c(Br)cccc1Nc1ncnc2cc3c(cc12)OC(CCN1CCOCC1)CO3]

Following general procedure GP-1, compound (±)-JGK064 was prepared from (±)-10 (35 mg, 0.07 mmol) and morpholine (31 μL, 0.35 mmol) in DMF (1.4 mL). PTLC (EtOAc, 0.5% acetonitrile, 1.5% aq. NH₄OH) followed by another PTLC (EtOAc) afforded (±)-JGK064 (25 mg, 73%) as an off-white, friable foam.

¹H NMR (500 MHz, CDCl₃): δ=8.68 (s, 1H), 8.65 (ddd, J=8.3, 7.4, 1.5 Hz, 1H), 7.39 (s, 1H), 7.36 (br, 1H), 7.28 (s, 1H), 7.30-7.25 (m, 1H), 7.11 (td, J=8.2, 1.5 Hz, 1H), 4.44 (dd, J=11.3, 2.3 Hz, 1H), 4.43-4.37 (m, 1H), 4.10 (dd, J=11.3, 7.7 Hz, 1H), 3.73 (t, J=4.7 Hz, 4H), 2.62 (ddt, J=12.5, 8.4, 3.9 Hz, 2H), 2.57-2.42 (m, 4H), 2.00-1.82 ppm (m, 2H). ¹³C NMR (126 MHz, CDCl₃): δ=155.86, 153.31, 150.13 (d, J_{CF}=242.3 Hz), 149.40, 146.67, 144.33, 128.66 (d, J_{CF}=10.4 Hz), 127.22, 125.33 (d, J_{CF}=4.6 Hz), 121.75, 114.21, 110.63, 108.58 (d, J_{CF}=19.2 Hz), 105.87, 72.20, 68.33, 67.06, 54.23, 53.86, 28.15 ppm. HRMS (ESI): m/z [M+H]⁺ calcd for C₂₂H₂₃BrFN₄O₃⁺, 489.0932; found, 489.0935.

### (±)-N-(3-Bromo-2-fluorophenyl)-7-[(piperidin-1-yl)methyl]-7,8-dihydro[1,4]dioxino[2,3-g]quinazolin-4-amine ((±)-JGK065)

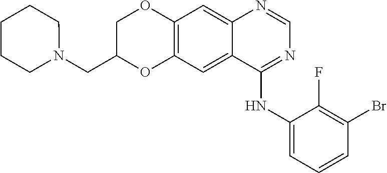 [SMILES: Fc1c(Br)cccc1Nc1ncnc2cc3c(cc12)OC(CN1CCCCC1)CO3]

Following general procedure GP-1, compound (±)-JGK065 was prepared from (±)-6 (40 mg, 0.08 mmol) and piperidine (41 μL, 0.41 mmol) in DMF (1.65 mL). PTLC (EtOAc) afforded (±)-JGK065 (24 mg, 61%) as an off-white, friable foam.

¹H NMR (500 MHz, CDCl₃): δ=8.66 (s, 1H), 8.63 (ddd, J=8.7, 7.3, 1.5 Hz, 1H), 7.369 (s, 1H), 7.368 (br, 1H), 7.30 (s, 1H), 7.26 (ddd, J=8.1, 6.5, 1.5 Hz, 1H), 7.09 (td, J=8.2, 1.5 Hz, 1H), 4.46 (dd, J=11.3, 2.3 Hz, 1H), 4.43 (ddd, J=8.3, 5.8, 2.0 Hz, 1H), 4.12 (dd, J=11.2, 7.5 Hz, 1H), 2.71 (dd, J=13.3, 5.9 Hz, 1H), 2.58 (dd, J=13.4, 6.2 Hz, 1H), 2.59-2.42 (m, 4H), 1.65-1.57 (m, 4H), 1.49-1.41 ppm (m, 2H). ¹³C NMR (126 MHz, CDCl₃): δ=155.86, 153.26, 150.12 (d, J_{CF}=242.6 Hz), 149.49, 146.62, 144.23, 128.65 (d, J_{CF}=10.3 Hz), 127.18, 125.27 (d, J_{CF}=4.5 Hz), 121.76, 114.16, 110.57, 108.56 (d, J_{CF}=19.4 Hz), 106.00, 71.87, 67.46, 59.34, 55.59, 26.07, 24.20 ppm. HRMS (ESI): m/z [M+H]⁺ calcd for C₂₂H₂₃BrFN₄O₂⁺, 473.0983; found, 473.0991.

### (±)-N-(3-Bromo-2-fluorophenyl)-7-[(dimethylamino)methyl]-7,8-dihydro[1,4]dioxino[2,3-g]quinazolin-4-amine ((±)-JGK066)

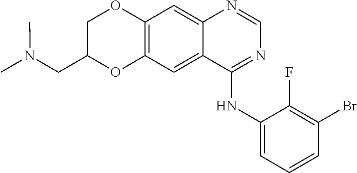 [SMILES: CN(C)CC1COc2cc3ncnc(Nc4cccc(Br)c4F)c3cc2O1]

Following general procedure GP-1, compound (±)-JGK066 was prepared from (±)-6 (45 mg, 0.09 mmol) and a 2 M solution of Me₂NH in THF (232 μL, 0.46 mmol) in DMF (1.85 mL). PTLC (EtOAc, 0.5% acetonitrile, 1.5% aq. NH₄OH) afforded (±)-JGK066 (39 mg, 97%) as an off-white, friable foam.

¹H NMR (500 MHz, CDCl₃): δ=8.680 (s, 1H), 8.675 (ddd, J=8.2, 7.5, 1.5 Hz, 1H), 7.39 (s, 1H), 7.38 (s, 1H), 7.37 (br, 1H), 7.27 (ddd, J=8.0, 6.4, 1.5 Hz, 1H), 7.10 (d, J=1.6 Hz, 1H), 4.46-4.41 (m, 1H), 4.45 (dd, J=11.8, 2.3 Hz, 1H), 4.12 (dd, J=11.9, 8.1 Hz, 1H), 2.73 (dd, J=13.2, 7.1 Hz, 1H), 2.55 (dd, J=13.1, 5.0 Hz, 1H), 2.38 ppm (s, 6H). ¹³C NMR (126 MHz, CDCl₃): δ=155.89, 153.34, 150.07 (d, J_{CF}=242.3 Hz), 149.38, 146.67, 144.06, 128.70 (d, J_{CF}=10.4 Hz), 127.16, 125.29 (d, J_{CF}=4.7 Hz), 121.65, 114.27, 110.67, 108.56 (d, J_{CF}=19.4 Hz), 106.15, 71.70, 67.20, 59.78, 46.41 ppm. HRMS (ESI): m/z [M+H]⁺ calcd for C₁₉H₁₉BrFN₄O₂⁺, 433.0670; found, 433.0677.

### (±)-N-(3-Bromo-2-fluorophenyl)-7-[(pyrrolidin-1-yl)methyl]-7,8-dihydro[1,4]dioxino[2,3-g]quinazolin-4-amine ((±)-JGK067)

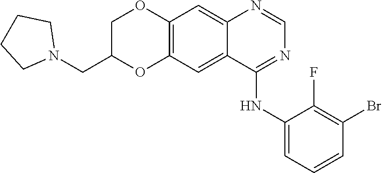 [SMILES: Fc1c(Br)cccc1Nc1ncnc2cc3c(cc12)OC(CN1CCCC1)CO3]

Following general procedure GP-1, compound (±)-JGK067 was prepared from (±)-6 (35 mg, 0.07 mmol) and pyrrolidine (30 μL, 0.36 mmol) in DMF (1.45 mL). PTLC (EtOAc, 1.5% iPrOH, 1.5% aq. NH₄OH) afforded (±)-JGK067 (31 mg, 93%) as an off-white, friable foam.

¹H NMR (500 MHz, CDCl₃): δ=8.68 (s, 1H), 8.67 (ddd, J=8.7, 7.5, 1.6 Hz, 2H), 7.39 (s, 1H), 7.36 (br, 1H), 7.35 (s, 1H), 7.27 (ddd, J=8.0, 6.4, 1.5 Hz, 2H), 7.10 (td, J=8.2, 1.5 Hz, 1H), 4.49-4.42 (m, 1H), 4.48 (dd, J=11.6, 2.0 Hz, 1H), 4.15 (dd, J=11.7, 8.0 Hz, 1H), 2.88 (dd, J=12.9, 6.5 Hz, 1H), 2.80 (dd, J=12.6, 5.5 Hz, 1H), 2.72-2.60 (m, 4H), 1.90-1.79 ppm (m, 4H). ¹³C NMR (126 MHz, CDCl₃): δ=155.87, 153.32, 150.09 (d, J_{CF}=242.6 Hz), 149.45, 146.68, 144.18, 128.71 (d, J_{CF}=10.3 Hz), 127.15, 125.30 (d, J_{CF}=4.7 Hz), 121.67, 114.26, 110.65, 108.56 (d, J_{CF}=19.4 Hz), 106.06, 72.73, 67.35, 56.57, 55.15, 23.75 ppm. HRMS (ESI): m/z [M+H]⁺ calcd for C₂₁H₂₁BrFN₄O₂⁺, 459.0826; found, 459.0845.

### (±)-N-(3-Bromo-2-fluorophenyl)-7- [(4-methylpiperazin-1-yl)methyl]-7,8-dihydro [1,4]dioxino[2,3-g]quinazolin-4-amine ((±)-JGK068)

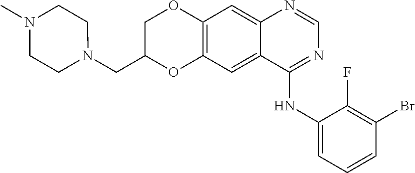 [SMILES: CN1CCN(CC2COc3cc4ncnc(Nc5cccc(Br)c5F)c4cc3O2)CC1]

Following general procedure GP-1, compound (±)-JGK068 was prepared from (±)-6 (35 mg, 0.07 mmol) and 1-methylpiperazine (40 μL, 0.36 mmol) in DMF (1.45 mL). PTLC (EtOAc/iPrOH 85:15, 1.5% aq. NH₄OH) afforded (±)-JGK068 (29 mg, 82%) as an off-white, friable foam.

¹H NMR (500 MHz, CDCl₃): δ=8.68 (s, 1H), 8.64 (ddd, J=8.3, 7.3, 1.5 Hz, 1H), 7.39 (s, 1H), 7.36 (br d, J=3.8 Hz, 1H), 7.32 (s, 1H), 7.27 (ddd, J=8.0, 6.5, 1.6 Hz, 1H), 7.10 (td, J=8.2, 1.5 Hz, 1H), 4.48-4.41 (m, 2H), 4.15 (dd, J=11.5, 8.6 Hz, 1H), 2.78 (dd, J=13.4, 6.0 Hz, 1H), 2.661 (dd, J=13.4, 5.8 Hz, 1H), 2.656 (br, 4H), 2.51 (br, 4H), 2.32 ppm (s, 3H). ¹³C NMR (126 MHz, CDCl₃): δ=155.89, 153.35, 150.15 (d, J_{CF}=242.6 Hz), 149.40, 146.69, 144.11, 128.64 (d, J_{CF}=10.3 Hz), 127.24, 125.30 (d, J_{CF}=4.7 Hz), 121.78, 114.27, 110.63, 108.59 (d, J_{CF}=19.2 Hz), 106.07, 71.80, 67.27, 58.43, 55.10, 53.96, 46.06 ppm. HRMS (ESI): m/z [M+H]⁺ calcd for C₂₂H₂₄BrFN₅O₂⁺, 488.1092; found, 488.1109.

### (±)-N-(3-Bromo-2-fluorophenyl)-7-[2-(dimethylamino)ethyl]-7,8-dihydro[1,4]dioxino[2,3-g]quinazolin-4-amine ((±)-JGK069)

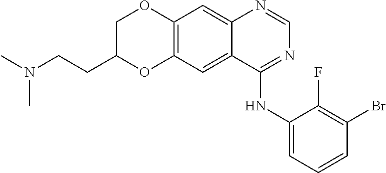 [SMILES: CN(C)CCC1COc2cc3ncnc(Nc4cccc(Br)c4F)c3cc2O1]

Following general procedure GP-1, compound (±)-JGK069 was prepared from (±)-10 (32 mg, 0.06 mmol) and a 2 M solution of Me₂NH in THF (161 μL, 0.32 mmol) in DMF (1.3 mL). PTLC (EtOAc, 5% iPrOH, 1.5% aq. NH₄OH) afforded (±)-JGK069 (19 mg, 66%) as an off-white friable foam.

¹H NMR (500 MHz, CDCl₃): δ=8.67 (s, 1H), 8.63 (ddd, J=8.7, 7.4, 1.6 Hz, 1H), 7.373 (br, 1H), 7.371 (s, 1H), 7.28 (s, 1H), 7.28-7.24 (m, 1H), 7.10 (td, J=8.2, 1.5 Hz, 1H), 4.42 (dd, J=11.4, 2.3 Hz, 1H), 4.38 (tdd, J=7.7, 5.1, 2.3 Hz, 1H), 4.08 (dd, J=11.3, 7.8 Hz, 1H), 2.56 (t, J=7.2 Hz, 2H), 2.29 (s, 6H), 1.93 (dq, J=14.2, 7.4 Hz, 1H), 1.84 ppm (dtd, J=14.2, 7.5, 5.1 Hz, 1H). ¹³C NMR (126 MHz, CDCl₃): δ=155.86, 153.26, 150.14 (d, J_{CF}=242.4 Hz), 149.42, 146.65, 144.36, 128.67 (d, J_{CF}=10.5 Hz), 127.18, 125.30 (d, J_{CF}=4.7 Hz), 121.77, 114.14, 110.60, 108.56 (d, J_{CF}=19.2 Hz), 105.88, 72.19, 68.34, 55.06, 45.58, 29.16 ppm. HRMS (ESI): m/z [M+H]⁺ calcd for C₂₀H₂₁BrFN₄O₂⁺, 447.0826; found, 447.0820.

### (±)-N-(3-Bromo-2-fluorophenyl)-7-[2-(4-methylpiperazin-1-yl)ethyl]-7,8-dihydro[1,4]dioxino[2,3-g]quinazolin-4-amine ((±)-JGK070)

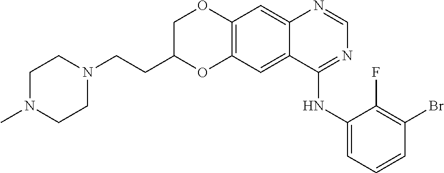 [SMILES: CN1CCN(CCC2COc3cc4ncnc(Nc5cccc(Br)c5F)c4cc3O2)CC1]

Following general procedure GP-1, compound (±)-JGK070 was prepared from (±)-10 (32 mg, 0.06 mmol) and 1-methylpiperazine (36 μL, 0.32 mmol) in DMF (1.3 mL). PTLC (EtOAc/iPrOH 8:2, 1.5% aq. NH₄OH) afforded (±)-JGK070 (21 mg, 65%) as an off-white friable foam.

¹H NMR (500 MHz, CDCl₃): δ=8.66 (s, 1H), 8.62 (ddd, J=8.5, 7.3, 1.5 Hz, 1H), 7.373 (br, 1H), 7.367 (s, 1H), 7.29-7.24 (m, 1H), 7.28 (s, 1H), 7.09 (td, J=8.2, 1.5 Hz, 1H), 4.43 (dd, J=11.4, 2.3 Hz, 1H), 4.37 (tdd, J=7.7, 5.4, 2.3 Hz, 1H), 4.08 (dd, J=11.4, 7.9 Hz, 1H), 2.68-2.54 (m, 2H), 2.50 (br, 8H), 2.30 (s, 3H), 1.94 (dtd, J=13.6, 7.5, 6.0 Hz, 1H), 1.86 ppm (dtd, J=14.2, 7.3, 5.3 Hz, 1H). ¹³C NMR (126 MHz, CDCl₃): δ=155.86, 153.27, 150.16 (d, J_{CF}=242.5 Hz), 149.41, 146.64, 144.37, 128.64 (d, J_{CF}=10.3 Hz), 127.22, 125.28 (d, J_{CF}=4.6 Hz), 121.81, 114.13, 110.60, 108.57 (d, J_{CF}=19.4 Hz), 105.88, 72.38, 68.36, 55.20, 53.77, 53.25, 46.11, 28.50 ppm. HRMS (ESI): m/z [M+H]⁺ calcd for C₂₃H₂₆BrFN₅O₂⁺, 502.1248; found, 502.1261.

### 5-Fluoro-2,3-dihydro-1,4-benzodioxine (11)

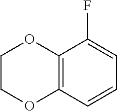 [SMILES: Fc1cccc2c1OCCO2]

A mixture of 3-fluorobenzene-1,2-diol (7233 mg, 56.5 mmol) in DMF (113 mL) was treated with K₂CO₃ (19514 mg, 141 mmol), stirred for 10 min at 23° C., and treated with 1-bromo-2-chloroethane (9.4 mL, 113 mmol). The mixture was stirred at 23° C. for 1 h, and then at 95° C. for 16 h. The mixture was cooled to 23° C., diluted with water (150 mL), and extracted with EtOAc (3×150 mL). The combined organics were washed with water (90 mL), brine (90 mL), dried (Na₂SO₄), filtered, and evaporated. FC (hexanes/EtOAc 30:1→10:1) afforded the title compound 11 (7973 mg, 92%) as a clear, colorless oil.

¹H NMR (400 MHz, CDCl₃): δ=6.78-6.63 (m, 3H), 4.34-4.26 ppm (m, 4H). ¹³C NMR (101 MHz, CDCl₃): δ=152.05 (d, J_{CF}=244.3 Hz), 145.27 (d, J_{CF}=3.8 Hz), 132.78 (d, J_{CF}=13.9 Hz), 120.02 (d, J_{CF}=8.9 Hz), 112.74 (d, J_{CF}=3.1 Hz), 108.52 (d, J_{CF}=18.1 Hz), 64.50, 64.45 ppm. HRMS (DART): m/z [M]·⁺ calcd for C₈H₇FO₂·⁺, 154.0425; found, 154.0420.

### 6-Bromo-5-fluoro-2,3-dihydro-1,4-benzodioxine (12)

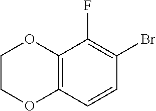 [SMILES: Fc1c(Br)ccc2c1OCCO2]

A solution of 11 (7812 mg, 50.7 mmol) in MeOH (101 mL) was treated with NBS (9022 mg, 50.7 mmol), and heated at 70° C. for 30 min. The mixture was cooled to 23° C., and concentrated. The residue was dissolved in DCM (700 mL), washed with water (300 mL), dried (MgSO₄), filtered, and evaporated. FC (hexanes/EtOAc 30:1→20:1) followed by drying under HV at 100° C. to remove any remaining starting material, afforded the title compound 12 (8807 mg, 75%, containing about 15% of the regioisomer) as a clear, colorless oil, which solidified in the freezer to give an off-white solid.

¹H NMR (400 MHz, CDCl₃): δ=6.96 (dd, J=9.0, 7.0 Hz, 1H), 6.59 (dd, J=9.0, 2.0 Hz, 1H), 4.35-4.24 ppm (m, 4H). ¹³C NMR (101 MHz, CDCl₃): δ=148.87 (d, J_{CF}=245.1 Hz), 144.53 (d, J_{CF}=3.5 Hz), 133.81 (d, J_{CF}=14.6 Hz), 123.31, 113.39 (d, J_{CF}=3.6 Hz), 109.17 (d, J_{CF}=19.3 Hz), 64.51, 64.34 ppm. HRMS (DART): m/z [M]·⁺ calcd for C₈H₆BrFO₂·⁺, 231.9530; found, 231.9525.

### 5-Fluoro-2,3-dihydro-1,4-benzodioxine-6-carboxylic acid (13)

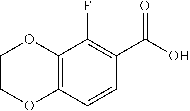 [SMILES: O=C(O)c1ccc2c(c1F)OCCO2]

A mixture of 12 (7.0 g, 30.0 mmol) in THF (108 mL) was cooled to −78° C., and treated dropwise with a 2.5 M solution of nBuLi in hexanes (12.02 mL, 30.0 mmol) during 10 min. The mixture was stirred at −78° C. for 30 min, and then transferred via cannula onto crushed dry ice (rinsed the cannula with 10 mL of THF). The mixture was allowed to warm to 23° C., and concentrated. Water (200 mL) and 1 M NaOH (50 mL) were added to the residue, and the aq. phase was extracted with Et₂O (3×60 mL). The aq. phase was acidified with 6 M HCl (15 mL), and extracted with DCM (3×150 mL). The combined organics were washed with brine (150 mL), dried (MgSO₄), filtered, and evaporated. FC (hexanes/EtOAc 7:3→3:7) afforded the title compound 13 (3591 mg, 60%) as a white solid.

¹H NMR (400 MHz, DMSO-d₆): δ=12.90 (br, 1H), 7.33 (dd, J=8.9, 7.7 Hz, 1H), 6.78 (dd, J=8.9, 1.7 Hz, 1H), 4.39-4.29 ppm (m, 4H). ¹³C NMR (101 MHz, DMSO-d₆): δ=164.65 (d, J_{CF}=3.0 Hz), 151.21 (d, J_{CF}=257.5 Hz), 148.50 (d, J_{CF}=4.4 Hz), 132.68 (d, J_{CF}=13.6 Hz), 122.44 (d, J_{CF}=1.4 Hz), 112.12 (d, J_{CF}=3.4 Hz), 111.97 (d, J_{CF}=7.3 Hz), 64.42, 63.91 ppm. HRMS (DART): m/z [M−H]⁻ calcd for C₉H₆FO₄⁻, 197.0256; found, 197.0250.

### Ethyl (5-fluoro-2,3-dihydro-1,4-benzodioxin-6-yl)carbamate (14)

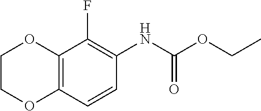 [SMILES: CCOC(=O)Nc1ccc2c(c1F)OCCO2]

A mixture of 13 (650 mg, 3.28 mmol) in toluene (13.1 mL) was treated with Et₃N (1.4 mL, 9.84 mmol), and at 10° C. with DPPA (780 μL, 3.62 mmol). The mixture was stirred at 23° C. for 30 min, then at 85° C. for 1.5 h. The mixture was cooled to 23° C., treated with EtOH (5 mL), stirred for 1.5 h at 23° C., and concentrated. The residue was dissolved in Et₂O (150 mL), washed with sat. aq. NaHCO₃ (40 mL), water (40 mL), brine (40 mL), dried (MgSO₄), filtered, and evaporated. FC (hexanes/DCM 7:3→1:9) afforded the title compound 14 (512 mg, 65%) as a white solid.

¹H NMR (500 MHz, CDCl₃): δ=7.42 (br, 1H), 6.64 (dd, J=9.2, 2.2 Hz, 1H), 6.56 (br, 1H), 4.32-4.24 (m, 4H), 4.22 (q, J=7.1 Hz, 2H), 1.31 ppm (t, J=7.1 Hz, 3H). ¹³C NMR (126 MHz, CDCl₃): δ=153.80, 142.61 (d, J_{CF}=246.0 Hz), 140.82, 132.66 (d, J_{CF}=12.4 Hz), 120.36 (d, J_{CF}=6.9 Hz), 112.36, 111.81 (d, J_{CF}=3.7 Hz), 64.72, 64.29, 61.61, 14.66 ppm. HRMS (DART): m/z [M+H]⁺ calcd for C₁₁H₁₃FNO₄⁺, 242.0823; found, 242.0816.

### 10-Fluoro-7,8-dihydro[1,4]dioxino[2,3-g]quinazoline (15)

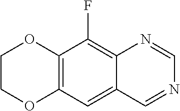 [SMILES: Fc1c2c(cc3cncnc13)OCCO2]

A mixture of 14 (450 mg, 1.87 mmol) and HMTA (263 mg, 1.87 mmol) in TFA (5.7 mL) was irradiated in the microwave at 110° C. for 10 min. The mixture was cooled to 23° C., diluted with water (60 mL), treated with 6 M NaOH (12 mL), and extracted with DCM (3×60 mL). The combined organics were washed with water (50 mL), brine (50 mL), dried (Na₂SO₄), filtered, and evaporated to give a foamy, yellow oil.

A mixture of the oil in 10% KOH in dioxane/water 1:1 (15.5 mL) was treated with [K₃Fe(CN)₆] (614 mg, 1.87 mmol), and irradiated in the microwave at 100° C. for 10 min This procedure was repeated a total of four times (4 cycles of addition of 1 equiv of potassium ferricyanide followed by microwave irradiation). The resulting mixture was diluted with water (160 mL), and extracted with DCM (3×120 mL). The combined organics were washed with water (100 mL), brine (100 mL), dried (Na₂SO₄), filtered, and evaporated to afford the title compound 15 (330 mg, 86%) as a yellow solid, which was used in the next step without any further purification.

¹H NMR (500 MHz, CDCl₃): δ=9.21 (br, 1H), 9.19 (s, 1H), 7.18 (d, J=2.0 Hz, 1H), 4.53-4.41 ppm (m, 4H). ¹³C NMR (126 MHz, CDCl₃): δ=158.06 (d, J_{CF}=2.9 Hz), 153.81 (d, J_{CF}=1.8 Hz), 145.76 (d, J_{CF}=2.9 Hz), 144.40 (d, J_{CF}=256.1 Hz), 138.56 (d, J_{CF}=11.0 Hz), 136.73 (d, J_{CF}=10.1 Hz), 119.81 (d, J_{CF}=2.7 Hz), 106.55 (d, J_{CF}=4.3 Hz), 64.78, 64.34 ppm. HRMS (DART): m/z [M+H]⁺ calcd for C₁₀H₈FN₂O₂⁺, 207.0564; found, 207.0563.

### 10-Fluoro-7,8-dihydro[1,4]dioxino[2,3-g]quinazolin-4(3H)-one (16)

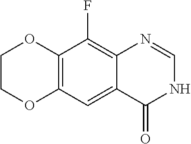 [SMILES: O=c1[nH]cnc2c(F)c3c(cc12)OCCO3]

A solution of 15 (306 mg, 1.48 mmol) in AcOH (1 mL) was treated dropwise with a 0.833 M solution of CAN in water (7.12 mL, 5.94 mmol), and stirred at 23° C. for 15 min. The white precipitate was collected by filtration, and washed with water (2×2 mL), acetonitrile (2×2 mL), DCM (2 mL), and Et₂O (2 mL) to afford a first batch of the title compound. The aq. filtrate was neutralized to pH ˜7 with 1 M NaOH, and the white precipitate was collected as before by filtration, followed by washings to afford a second batch of the title compound 16 (81 mg, 25%) as a white solid.

¹H NMR (500 MHz, DMSO-d₆): δ=12.19 (br, 1H), 7.98 (d, J=3.3 Hz, 1H), 7.32 (s, 1H), 4.52-4.28 ppm (m, 4H). ¹³C NMR (126 MHz, DMSO-d₆): δ=159.31, 144.58 (d, J_{CF}=251.8 Hz), 144.28, 143.80 (d, J_{CF}=3.4 Hz), 137.86 (d, J_{CF}=11.1 Hz), 132.94 (d, J_{CF}=8.9 Hz), 115.75, 106.62 (d, J_{CF}=3.7 Hz), 64.57, 64.02 ppm. HRMS (DART): m/z [M+H]⁺ calcd for C₁₀H₈FN₂O₃⁺, 223.0513; found, 223.0503.

### 4-Chloro-10-fluoro-7,8-dihydro[1,4]dioxino[2,3-g]quinazoline (17)

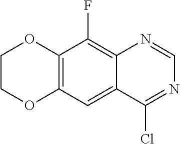 [SMILES: Fc1c2c(cc3c(Cl)ncnc13)OCCO2]

A stirred suspension of 16 (92 mg, 0.41 mmol) in toluene (1.2 mL) was treated with DIPEA (220 μL, 1.26 mmol), followed by dropwise addition of POCl₃ (103 μL, 1.12 mmol) at 10° C. The mixture was stirred at 23° C. for 1 h, then at 90° C. for 5 h, and concentrated. The residue was treated with sat. aq. NaHCO₃ (10 mL) at 0° C. for 5 min, diluted with water (5 mL), and extracted with DCM (3×7 mL). The combined organics were washed with half-sat. aq. NaHCO₃ (7 mL), brine (7 mL), dried (Na₂SO₄), filtered, and evaporated to afford the title compound 17 (51 mg, 51%) as a light-brown solid, which was used in the next step without any further purification.

¹H NMR (500 MHz, CDCl₃): δ=8.90 (s, 1H), 7.51 (d, J=2.0 Hz, 1H), 4.55-4.43 ppm (m, 4H). ¹³C NMR (126 MHz, CDCl₃): δ=160.48 (d, J_{CF}=4.3 Hz), 152.31, 146.29 (d, J_{CF}=3.3 Hz), 144.63 (d, J_{CF}=256.2 Hz), 138.95 (d, J_{CF}=11.3 Hz), 137.68 (d, J_{CF}=10.2 Hz), 118.56 (d, J_{CF}=2.4 Hz), 105.82 (d, J_{CF}=4.2 Hz), 64.81, 64.41 ppm. HRMS (DART): m/z [M+H]⁺ calcd for C₁₀H₇ClFN₂O₂⁺, 241.0175; found, 241.0174.

### 5-Fluoro-7-nitro-2,3-dihydro-1,4-benzodioxine-6-carboxylic acid (18)

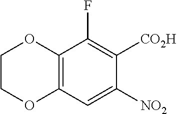 [SMILES: O=C(O)c1c([N+](=O)[O-])cc2c(c1F)OCCO2]

A mixture of 13 (1500 mg, 7.57 mmol) in AcOH (7.5 mL) was treated dropwise with H₂SO₄ (2.02 mL) at 10° C. The vigorously stirred mixture was treated dropwise with 65% HNO₃ (2.6 mL) at 0° C. during 10 min. The resulting mixture was stirred at 0° C. for 30 min, and then at 23° C. for 16 h. The mixture was poured into ice-water (40 mL), and the white precipitate was collected by filtration (washings with cold water, 40 mL), and dried in a desiccator to afford the title compound 18 (1280 mg, 70%) as a white solid.

¹H NMR (500 MHz, DMSO-d₆): δ=14.09 (br, 1H), 7.62 (d, J=1.7 Hz, 1H), 4.52-4.40 ppm (m, 4H). ¹³C NMR (126 MHz, DMSO-d₆): δ=162.71, 147.16 (d, J_{CF}=248.7 Hz), 144.72 (d, J_{CF}=5.1 Hz), 138.15 (d, J_{CF}=13.7 Hz), 137.10 (d, J_{CF}=6.6 Hz), 113.44 (d, J_{CF}=20.3 Hz), 109.52 (d, J_{CF}=2.3 Hz), 64.97, 64.48 ppm. HRMS (DART): m/z [M−H]⁻ calcd for C₉H₅FNO₆⁻, 242.0106; found, 242.0124.

### 7-Amino-5-fluoro-2,3-dihydro-1,4-benzodioxine-6-carboxylic acid (19)

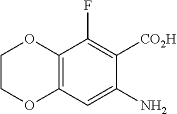 [SMILES: Nc1cc2c(c(F)c1C(=O)O)OCCO2]

A mixture of 18 (500 mg, 2.06 mmol) and 5% Pd/C (223 mg, 0.10 mmol) in MeOH (21 mL) was stirred under an atmosphere of H₂ at 23° C. for 13.5 h. The mixture was filtered through Celite (washings with EtOH), and evaporated to give the title compound 19 (418 mg, 95%) as a grey solid, which did not seem to be very stable.

¹H NMR (500 MHz, DMSO-d₆): δ=8.35 (br, 2H), 6.04 (d, J=1.9 Hz, 1H), 4.29-4.24 (m, 2H), 4.19-4.14 ppm (m, 2H). ¹³C NMR (126 MHz, DMSO-d₆): δ=167.36 (d, J_{CF}=2.9 Hz), 151.36 (d, J_{CF}=252.0 Hz), 148.86 (d, J_{CF}=7.0 Hz), 145.81 (d, J_{CF}=5.7 Hz), 122.88 (d, J_{CF}=15.4 Hz), 97.19 (d, J_{CF}=2.9 Hz), 95.37 (d, J_{CF}=10.9 Hz), 64.95, 63.58 ppm. HRMS (DART): m/z [M+H]⁺ calcd for C₉H₉FNO₄⁺, 214.0510; found, 214.0508.

### 5-Fluoro-7,8-dihydro[1,4]dioxino[2,3-g]quinazolin-4(3H)-one (20)

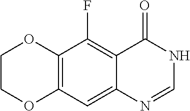 [SMILES: O=c1[nH]cnc2cc3c(c(F)c12)OCCO3]

A mixture of 19 (417 mg, 1.96 mmol) in formamide (2.3 mL, 58.7 mmol) was stirred at 120-125° C. for 16 h. The mixture was cooled to 0° C., and treated with water (4 mL), stirred for 30 min, diluted with water (4 mL), and filtered. The residue was washed with cold water (3×5 mL), and dried over Drierite under HV to afford the title compound 20 (249 mg, 57%) as an off-white solid.

¹H NMR (400 MHz, DMSO-d₆): δ=12.00 (br, 1H), 7.90 (d, J=3.6 Hz, 1H), 6.93 (d, J=1.9 Hz, 1H), 4.45-4.35 ppm (m, 4H). ¹³C NMR (126 MHz, DMSO-d₆): δ=157.64 (d, J_{CF}=3.0 Hz), 149.70 (d, J_{CF}=6.0 Hz), 148.45 (d, J_{CF}=261.3 Hz), 144.60, 142.99, 131.47 (d, J_{CF}=12.7 Hz), 108.76 (d, J_{CF}=3.5 Hz), 106.38 (d, J_{CF}=3.8 Hz), 64.69, 63.98 ppm. HRMS (DART): m/z [M+H]⁺ calcd for C₁₀H₈FN₂O₃⁺, 223.0513; found, 223.0510.

### 4-Chloro-5-fluoro-7,8-dihydro[1,4]dioxino[2,3-g]quinazoline (21)

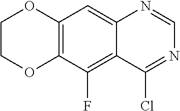 [SMILES: Fc1c2c(cc3ncnc(Cl)c13)OCCO2]

A stirred suspension of 20 (90 mg, 0.41 mmol) in toluene (1.2 mL) was treated with DIPEA (215 μL, 1.24 mmol), followed by dropwise addition of POCl₃ (100 μL, 1.09 mmol) at 10° C. The mixture was stirred at 23° C. for 1 h, then at 88° C. for 5 h, and concentrated. The residue was treated with sat. aq. NaHCO₃ (10 mL) at 0° C., diluted with water (5 mL), and extracted with DCM (3×7 mL). The combined organics were washed with half-sat. aq. NaHCO₃ (7 mL), brine (7 mL), dried (Na₂SO₄), filtered, and evaporated to afford the title compound 21 (96 mg, 99%) as a light-orange solid, which was used in the next step without any further purification.

¹H NMR (500 MHz, CDCl₃): δ=8.83 (s, 1H), 7.35 (d, J=2.0 Hz, 1H), 4.51-4.45 ppm (m, 4H). ¹³C NMR (126 MHz, CDCl₃): δ=156.76 (d, J_{CF}=4.5 Hz), 152.70 (d, J_{CF}=2.3 Hz), 151.66 (d, J_{CF}=4.9 Hz), 146.08, 144.51 (d, J_{CF}=261.8 Hz), 134.04 (d, J_{CF}=14.0 Hz), 110.85 (d, J_{CF}=7.7 Hz), 109.43 (d, J_{CF}=4.0 Hz), 64.89, 64.37 ppm. HRMS (DART): m/z [M+H]⁺ calcd for C₁₀H₇ClFN₂O₂⁺, 241.0175; found, 241.0176.

### N-(3-Bromo-2-fluorophenyl)-10-fluoro-7,8-dihydro[1,4]dioxino[2,3-g]quinazolin-4-amine (JGK071)

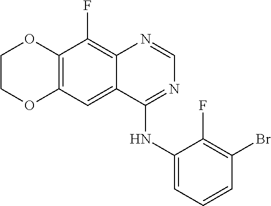 [SMILES: Fc1c(Br)cccc1Nc1ncnc2c(F)c3c(cc12)OCCO3]

Following general procedure GP-2, compound JGK071 was prepared from chloroquinazoline 17 (35 mg, 0.15 mmol) and 3-bromo-2-fluoroaniline. FC (DCM/EtOAc 1:0→8:2) afforded JGK071 (44 mg, 77%) as a white solid.

¹H NMR (500 MHz, DMSO-d₆): δ=9.76 (s, 1H), 8.38 (s, 1H), 7.80 (d, J=1.8 Hz, 1H), 7.62 (ddd, J=8.0, 6.3, 1.6 Hz, 1H), 7.54 (ddd, J=8.5, 7.1, 1.6 Hz, 1H), 7.22 (td, J=8.0, 1.2 Hz, 1H), 4.53-4.40 ppm (m, 4H). ¹³C NMR (126 MHz, DMSO-d₆): δ=156.93 (d, J_{CF}=3.7 Hz), 153.44 (d, J_{CF}=247.5 Hz), 153.12, 144.04 (d, J_{CF}=250.0 Hz), 143.97 (d, J_{CF}=3.2 Hz), 137.04 (d, J_{CF}=10.9 Hz), 135.62 (d, J_{CF}=9.9 Hz), 130.48, 127.89, 127.62 (d, J_{CF}=13.1 Hz), 125.51 (d, J_{CF}=4.5 Hz), 108.58 (d, J_{CF}=23.4 Hz), 108.51, 103.25 (d, J_{CF}=3.9 Hz), 64.63, 64.21 ppm. HRMS (DART): m/z [M+H]⁺ calcd for C₁₆H₁₁BrF₂N₃O₂⁺, 393.9997; found, 393.9999.

### N-(3-Bromo-2-fluorophenyl)-5-fluoro-7,8-dihydro[1,4]dioxino[2,3-g]quinazolin-4-amine (JGK072)

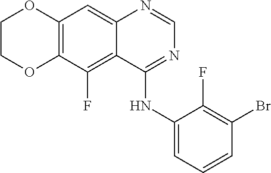 [SMILES: Fc1c(Br)cccc1Nc1ncnc2cc3c(c(F)c12)OCCO3]

Following general procedure GP-2, compound JGK072 was prepared from chloroquinazoline 21 (35 mg, 0.15 mmol) and 3-bromo-2-fluoroaniline. FC (DCM/EtOAc 1:0→8:2) afforded JGK072 (47 mg, 82%) as a white solid.

¹H NMR (500 MHz, CDCl₃): δ=8.67 (ddd, J=8.6, 7.2, 1.5 Hz, 1H), 8.62 (s, 1H), 8.52 (dd, J=19.6, 2.2 Hz, 1H), 7.29 (ddd, J=8.1, 6.4, 1.5 Hz, 1H), 7.23 (d, J=2.0 Hz, 1H), 7.10 (td, J=8.2, 1.6 Hz, 1H), 4.48-4.42 ppm (m, 4H). ¹³C NMR (126 MHz, CDCl₃): δ=155.27 (d, J_{CF}=5.2 Hz), 153.90, 150.34 (d, J_{CF}=243.9 Hz), 149.93 (d, J_{CF}=6.2 Hz), 145.75 (d, J_{CF}=250.3 Hz), 144.78, 131.96 (d, J_{CF}=15.6 Hz), 128.43 (d, J_{CF}=10.4 Hz), 127.71, 125.20 (d, J_{CF}=4.7 Hz), 122.48, 109.69 (d, J_{CF}=3.3 Hz), 108.63 (d, J_{CF}=19.2 Hz), 101.42 (d, J_{CF}=7.2 Hz), 64.84, 64.48 ppm. HRMS (DART): m/z [M+H]⁺ calcd for C₁₆H₁₁BrF₂N₃O₂⁺, 393.9997; found, 393.9996.

### Preparation of Further Exemplary Compounds of the JGK Series

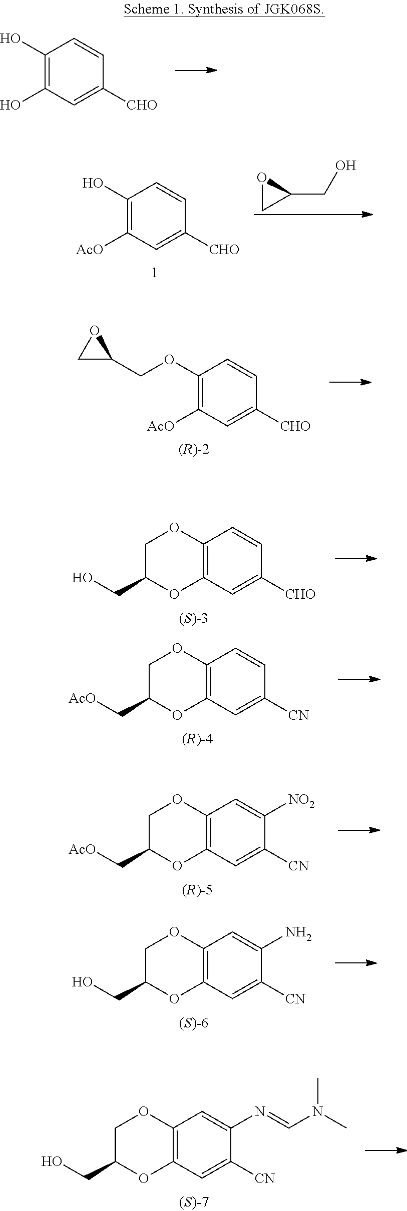

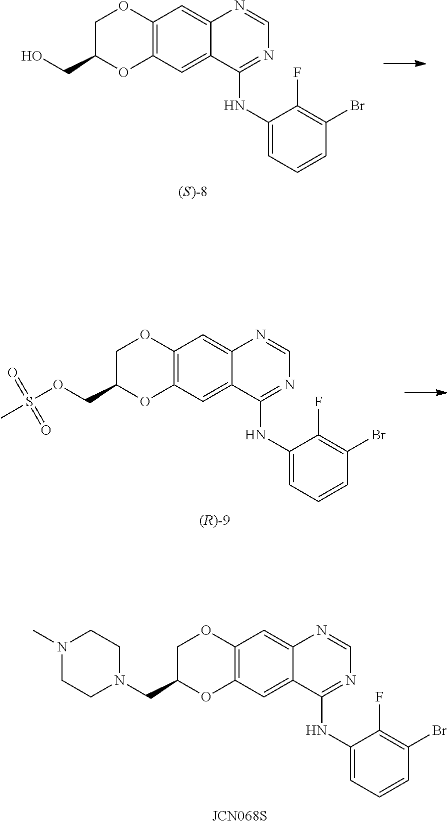 [SMILES: OC[C@H]1COc2cc3ncnc(Nc4cccc(Br)c4F)c3cc2O1 ; CS(=O)(=O)OC[C@H]1COc2cc3ncnc(Nc4cccc(Br)c4F)c3cc2O1 ; CN1CCN(C[C@H]2COc3cc4ncnc(Nc5cccc(Br)c5F)c4cc3O2)CC1]

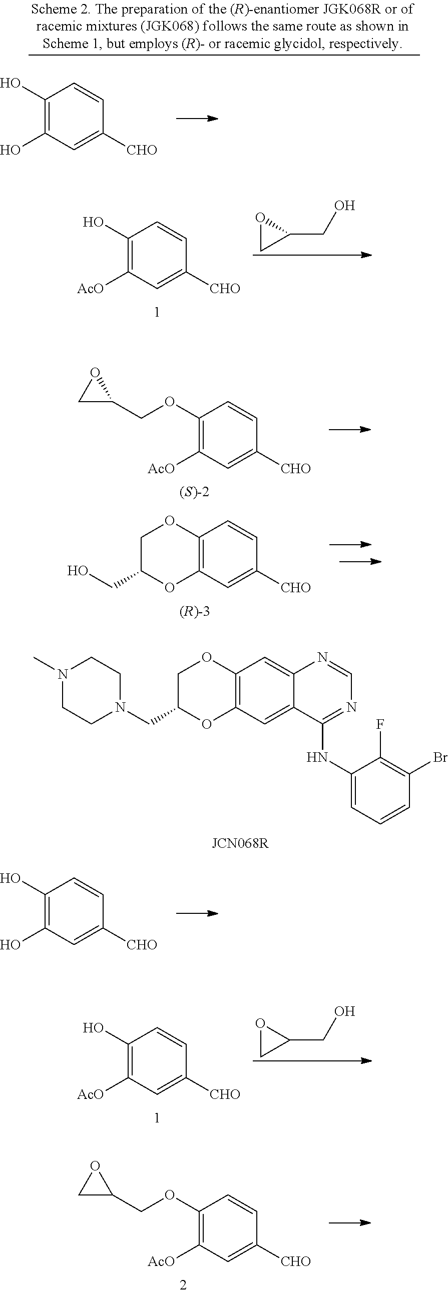

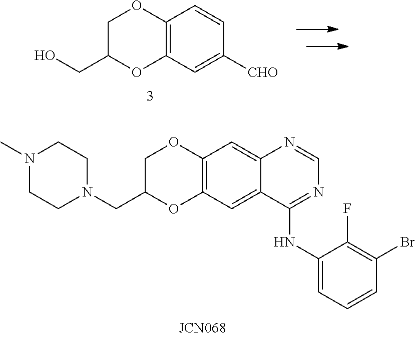 [SMILES: O=Cc1ccc2c(c1)OC(CO)CO2 ; CN1CCN(CC2COc3cc4ncnc(Nc5cccc(Br)c5F)c4cc3O2)CC1]

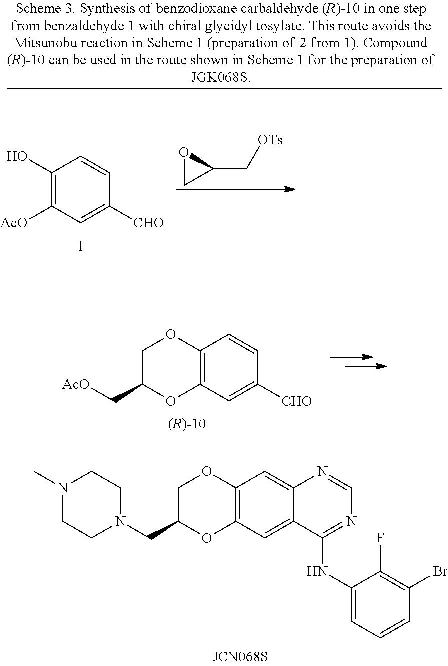 [SMILES: CC(=O)Oc1cc(C=O)ccc1O ; Cc1ccc(S(=O)(=O)OC[C@H]2CO2)cc1 ; CC(=O)OC[C@H]1COc2ccc(C=O)cc2O1 ; CN1CCN(C[C@H]2COc3cc4ncnc(Nc5cccc(Br)c5F)c4cc3O2)CC1]

### 5-Formyl-2-hydroxyphenyl acetate (1)

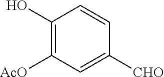 [SMILES: CC(=O)Oc1cc(C=O)ccc1O]

A mixture of 3,4-dihydroxybenzaldehyde (100 g, 0.724 mol) in THF (965 mL) was cooled to 0° C., and treated with 10% aq. NaOH (724 mL, 1.81 mol) over 4-5 min. After the reaction mixture was stirred at 0° C. for 15 min, acetic anhydride (Ac₂O, 82.1 mL, 0.869 mol) was added dropwise over 20 min The mixture was stirred for 30 min at the same temperature, and then poured into a mixture of EtOAc (1.25 L) and 2 M HCl (1.13 L) at 0° C. The phases were separated, and the aq. phase was extracted with EtOAc (4×250 mL). The combined organics were washed with water (2×500 mL), brine (500 mL), dried (Na₂SO₄), filtered, and evaporated. The residue was treated with a small amount of n-heptane and evaporated (3×). Recrystallization from EtOAc (275 mL; crystals washed with Et₂O) gave a first crop of the title compound 1 (66.96 g, 51%) as light-brown crystals. Recrystallization of the mother liquor from EtOAc gave a second crop of the title compound 1 (29.436 g, 23%) as a light-brown solid. ¹H NMR (500 MHz, CDCl₃): δ9.85 (s, 1H), 7.73-7.65 (m, 2H), 7.11 (d, J=8.8 Hz, 1H), 6.34 (br, 1H), 2.39 (s, 3H). ¹³C NMR (126 MHz, CDCl₃): δ190.40, 168.99, 152.96, 138.81, 130.24, 129.72, 124.13, 117.87, 21.09. HRMS (DART): m/z [M+H]⁺ calcd for C₉H₉O₄⁺, 181.0495; found, 181.0488.

### 5-Formyl-2-{[(2R)-oxiran-2-yl]methoxy}phenyl acetate ((R)-2)

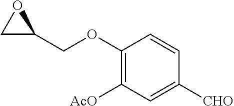 [SMILES: CC(=O)Oc1cc(C=O)ccc1OC[C@H]1CO1]

A mixture of 1 (32.5 g, 0.18 mol) and triphenylphosphine (PPh₃, 70.976 g, 0.27 mol) in THF (905 mL) was treated with (S)-glycidol (17.95 mL, 0.27 mol), cooled to 0° C., and treated dropwise with diisopropyl azodicarboxylate (DIAD, 56.8 mL, 0.289 mmol) over 30 min The mixture was stirred for an additional 10 min at 0° C., after which the cooling bath was removed, and stirring was continued at 23° C. for 15.5 h. All volatiles were evaporated, and crude (R)-2, obtained as a brown oil, was used without any further purification in the next step.

### (3S)-3-(Hydroxymethyl)-2,3-dihydro-1,4-benzodioxine-6-carbaldehyde ((S)-3)

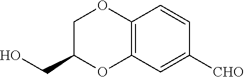 [SMILES: O=Cc1ccc2c(c1)O[C@@H](CO)CO2]

A mixture of crude (R)-2 in MeOH (1.564 L) was treated with K₂CO₃ (49.87 g, 0.36 mol) and stirred at 23° C. for 18.5 h, and then the solvent was evaporated. The residue was suspended in half-sat. NH₄Cl (750 mL), and extracted with EtOAc (3×500 mL). The combined organics were washed with water (250 mL), brine (250 mL), dried (Na₂SO₄), filtered, and evaporated. The crude material was purified by several rounds of flash chromatography (hexanes/EtOAc 9:1→1:1) as well as by precipitation from hexanes/Et₂O 1:1 (to remove triphenylphospine oxide Ph₃PO), to afford the title compound (S)-3 (17.322 g, 49% over two steps) as a white solid. ¹H NMR (500 MHz, CDCl₃): δ9.81 (s, 1H), 7.43 (d, J=1.8 Hz, 1H), 7.41 (dd, J=8.1, 1.9 Hz, 1H), 7.00 (d, J=8.2 Hz, 1H), 4.39 (dd, J=11.4, 2.3 Hz, 1H), 4.31-4.25 (m, 1H), 4.20 (dd, J=11.3, 7.9 Hz, 1H), 3.95 (dd, J=12.1, 4.3 Hz, 1H), 3.87 (dd, J=12.1, 4.9 Hz, 1H), 2.18 (br, 1H). ¹³C NMR (126 MHz, CDCl₃): δ190.79, 148.76, 143.46, 130.79, 124.46, 118.33, 117.70, 73.31, 65.61, 61.54. HRMS (DART): m/z [M+H]⁺ calcd for C₁₀H₁₁O₄⁺, 195.0652; found, 195.0650.

### [(2R)-7-Cyano-2,3-dihydro-1,4-benzodioxin-2-yl]methyl acetate ((R)-4)

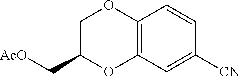 [SMILES: CC(=O)OC[C@H]1COc2ccc(C#N)cc2O1]

A mixture of (S)-3 (17.322 g, 0.089 mol) in AcOH (189 mL) was treated with KOAc (22.944 g, 0.234 mol), stirred at 23° C. for 10 min, and then treated with NH₂OH.HCl (16.233 g, 0.234 mol). The resulting mixture was stirred at 120-125° C. for 18.5 h. The mixture was cooled to 23° C., poured into water (1 L), and extracted with EtOAc (4×250 mL). The combined organics were treated with 3.5 M NaOH (400 mL) and sat. aq. NaHCO₃ (100 mL) to obtain a final pH of ˜8, and the emulsion was stirred at 23° C. for 1 h. The organic layer was separated, and washed with sat. aq. NaHCO₃ (300 mL), water (300 mL), brine (300 mL), dried (Na₂SO₄), filtered, and evaporated. Purification by flash chromatography (hexanes/EtOAc 10:1→6:4) afforded the title compound (R)-4 (13.513 g, 65%) as a clear, colorless oil. ¹H NMR (500 MHz, CDCl₃): δ7.20 (d, J=2.0 Hz, 1H), 7.16 (dd, J=8.4, 2.0 Hz, 1H), 6.94 (d, J=8.4 Hz, 1H), 4.43-4.38 (m, 1H), 4.36 (dd, J=11.6, 2.4 Hz, 1H), 4.34 (dd, J=11.1, 4.5 Hz, 1H), 4.30 (dd, J=11.6, 4.6 Hz, 1H), 4.11 (dd, J=11.5, 7.2 Hz, 1H), 2.12 (s, 3H). ¹³C NMR (126 MHz, CDCl₃): δ170.64, 147.13, 143.11, 126.28, 121.56, 118.85, 118.32, 105.13, 71.11, 65.45, 62.24, 20.83. HRMS (DART): m/z [M+H]⁺ calcd for C₁₂H₁₂NO₄⁺, 234.0761; found, 234.0759.

### [(2R)-7-Cyano-6-nitro-2,3-dihydro-1,4-benzodioxin-2-yl]methyl acetate ((R)-5)

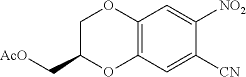 [SMILES: CC(=O)OC[C@H]1COc2cc([N+](=O)[O-])c(C#N)cc2O1]

A mixture of (R)-4 (13.345 g, 57.2 mmol) in Ac₂O (74.3 mL) was treated with H₂SO₄ (3.05 mL, 57.2 mmol), cooled to 0° C., and treated dropwise with 70% HNO₃ (19.63 mL, 286 mmol) at 0° C. over 35 min. The mixture was stirred for another 2 h at 0° C., and then at 23° C. for 3.5 h. The mixture was poured into ice-water (850 mL), and the pH was adjusted to ˜7 with 6 M NaOH (320 mL). Sat. aq. NaHCO₃ (200 mL) was added, and the mixture was extracted with CH₂Cl₂ (3×500 mL). The combined organics were washed with sat. aq. NaHCO₃ (400 mL), water (400 mL), brine (400 mL), dried (Na₂SO₄), filtered, and evaporated to afford the title compound (R)-5 (15.696 g, 99%) as a yellow oil, which was used in the next step without any further purification. ¹H NMR (500 MHz, DMSO-d₆): δ7.96 (s, 1H), 7.80 (s, 1H), 4.73-4.67 (m, 1H), 4.58 (dd, J=11.8, 2.6 Hz, 1H), 4.36 (dd, J=12.5, 3.7 Hz, 1H), 4.31 (dd, J=12.5, 5.7 Hz, 1H), 4.27 (dd, J=11.8, 7.0 Hz, 1H), 2.05 (s, 3H). ¹³C NMR (126 MHz, DMSO-d₆): δ170.11, 147.75, 146.26, 142.23, 123.77, 115.21, 115.17, 100.06, 72.00, 64.98, 61.72, 20.52. HRMS (DART): m/z [M+H]⁺ calcd for C₁₂H₁₁N₂O₆⁺, 279.0612; found, 279.0601.

### (3S)-7-Amino-3-(hydroxymethyl)-2,3-dihydro-1,4-benzodioxine-6-carbonitrile ((S)-6)

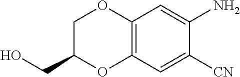 [SMILES: N#Cc1cc2c(cc1N)OC[C@H](CO)O2]

A suspension of (R)-5 (15.591 g, 56 mmol) in water/ethanol 1:1 (250 mL) was treated with Na₂S₂O₄ (39.266 g, 185 mmol), and the mixture was stirred at 50° C. for 105 min, and then heated to 70° C. for 2 h while treated portionwise with conc. HCl (73.6 mL , 0.897 mol) during that time. The mixture was cooled to 23° C., poured on ice, and the pH was adjusted to ˜10 with 6 M NaOH (200 mL) and half-sat. NaHCO₃ (500 mL). The mixture was extracted with EtOAc (3×500 mL). The combined organics were washed with water (500 mL), brine (500 mL), dried (Na₂SO₄), filtered, and evaporated to afford crude (S)-6 (9.483 g, 82%) as an orange-yellow solid, which was used in the next step without any further purification. ¹H NMR (500 MHz, DMSO-d₆): δ6.92 (s, 1H), 6.29 (s, 1H), 5.50 (br, 2H), 5.04 (t, J=5.7 Hz, 1H), 4.32 (dd, J=10.7, 1.6 Hz, 1H), 4.07-3.99 (m, 1H), 4.00 (dd, J=11.2, 8.3 Hz, 1H), 3.64-3.51 (m, 2H). ¹³C NMR (126 MHz, DMSO-d₆): δ148.50, 147.29, 134.39, 119.04, 118.04, 102.45, 86.72, 73.25, 65.92, 59.77. HRMS (DART): m/z [M+H]⁺ calcd for C₁₀H₁₀N₂O₃·⁺, 206.0686; found, 206.0685.

### N′-[(2S)-7-Cyano-2-(hydroxymethyl)-2,3-dihydro-1,4-benzodioxin-6-yl]-N,N-dimethylmethan-imidamide ((S)-7)

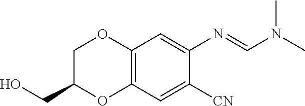 [SMILES: CN(C)/C=N/c1cc2c(cc1C#N)O[C@@H](CO)CO2]

A mixture of (S)-6 (9.38 g, 45.5 mmol) in toluene (117 mL) was treated with AcOH (143 μL, 2.5 mmol) and DMF-DMA (13.1 mL, 98.9 mmol), and the mixture was stirred at 105° C. for 3 h. The evaporated MeOH (˜4-5 mL) was collected in a Dean-Stark trap to monitor the progress of the reaction. The mixture was cooled to 23° C. and evaporated to obtain crude (S)-7 (14.243 g, quant.) as a viscous, brown oil, which was used in the next step without any further purification. ¹H NMR (500 MHz, CDCl₃): δ7.51 (s, 1H), 7.05 (s, 1H), 6.48 (s, 1H), 4.33 (dd, J=11.2, 2.0 Hz, 1H), 4.23-4.17 (m, 1H), 4.13 (dd, J=11.2, 8.1 Hz, 1H), 3.90 (dd, J=12.1, 4.2 Hz, 1H), 3.83 (dd, J=12.1, 4.8 Hz, 1H), 3.07 (s, 3H), 3.05 (s, 3H). ¹³C NMR (126 MHz, CDCl₃): δ160.40, 153.78, 147.68, 138.63, 121.08, 118.64, 108.16, 99.31, 73.34, 65.83, 61.67, 40.51, 34.82. HRMS (DART): m/z [M+H]⁺ calcd for C₁₃H₁₆N₃O₃⁺, 262.1186; found, 262.1183.

### [(7S)-4-(3-Bromo-2-fluoroanilino)-7,8-dihydro[1,4]dioxino[2,3-g]quinazolin-7-yl]methanol ((S)-8)

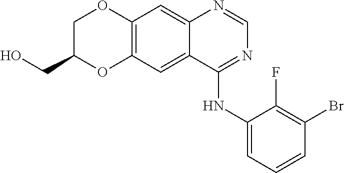 [SMILES: OC[C@H]1COc2cc3ncnc(Nc4cccc(Br)c4F)c3cc2O1]

A mixture of (S)-7 in AcOH (152 mL) was treated with 3-bromo-2-fluoroaniline (6.63 mL, 59.1 mmol), and the mixture was stirred at 125-130° C. for 3 h. The mixture was cooled to 23° C., poured into ice-water (500 mL), and the pH was adjusted to ˜9 with sat. aq. NH₄OH (185 mL) and half-sat. aq. NaHCO₃ (200 mL). The mixture was extracted with EtOAc (3×400 mL), and the combined organics were washed with half-sat. aq. NaHCO₃ (400 mL), water (400 mL), brine (400 mL), dried (Na₂SO₄), filtered, and evaporated. The residue was dissolved in MeOH (272 mL), and treated with K₂CO₃ (12.579 g, 91 mmol), stirred at 23° C. for 1 h, and evaporated. The residue was suspended in half-sat. aq. NH₄Cl (700 mL), and extracted with EtOAc (3×400 mL). The combined organics were washed with water (400 mL), brine (400 mL), dried (Na₂SO₄), filtered, and evaporated. The orange-yellow residue was suspended in EtOAc, warmed to 65° C., and then let slowly cool down to 23° C. overnight. Filtration, and washing of the residue with cold hexanes (2×15 mL) and Et₂O (2×15 mL), and drying under high vacuum afforded the title compound (S)-8 (9.407 g, 50.9% over two steps) as a fine, yellow powder. ¹H NMR (500 MHz, DMSO-d₆): δ9.69 (s, 1H), 8.33 (s, 1H), 7.99 (s, 1H), 7.59 (ddd, J=8.0, 6.2, 1.6 Hz, 1H), 7.54 (ddd, J=8.4, 7.0, 1.6 Hz, 1H), 7.24-7.17 (m, 1H), 7.20 (s, 1H), 5.20 (t, J=5.6 Hz, 1H), 4.49 (dd, J=11.5, 2.4 Hz, 1H), 4.37-4.29 (m, 1H), 4.21 (dd, J=11.6, 7.4 Hz, 1H), 3.76-3.64 (m, 2H). ¹³C NMR (126 MHz, DMSO-d₆): δ157.22, 153.38 (d, J_{CF}=247.0 Hz), 153.09, 148.87, 145.94, 143.37, 130.08, 128.09 (d, J_{CF}=12.9 Hz), 127.75, 125.43 (d, J_{CF}=4.5 Hz), 112.29, 109.81, 108.54 (d, J_{CF}=20.0 Hz), 108.49, 73.77, 65.52, 59.76. HRMS (DART): m/z [M+H]⁺ calcd for C₁₇H₁₄BrFN₃O₃⁺, 406.0197; found, 406.0185.

### [(7R)-4-(3-Bromo-2-fluoroanilino)-7,8-dihydro[1,4]dioxino[2,3-g]quinazolin-7-yl]methyl methanesulfonate ((R)-9)

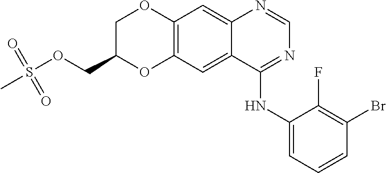 [SMILES: CS(=O)(=O)OC[C@H]1COc2cc3ncnc(Nc4cccc(Br)c4F)c3cc2O1]

A mixture of (S)-8 (9.01 g, 22.2 mmol) and Me₃N.HCl (234 mg, 2.45 mmol) in acetonitrile (148 mL) was treated with Et₃N (6.18 mL, 44.4 mmol), cooled to 0-5° C., and treated dropwise with a solution of MsCl (2.57 mL, 33.2 mmol) in acetonitrile (17 mL; rinsed with 3 mL) over 10 min. The mixture was stirred at 0° C. for 1 h. Water (100 mL) was added, and most of the acetonitrile was evaporated in vacuo. Additional water (700 mL) was added, and the mixture was extracted with EtOAc (3×400 mL). The combined organics were washed with water (400 mL), brine (400 mL), dried (Na₂SO₄), filtered, and evaporated to afford the title compound (R)-9 (10.33 g, 96%) as a yellow, friable foam, which was used in the next step without any further purification. ¹H NMR (500 MHz, CDCl₃): δ8.70 (s, 1H), 8.62 (ddd, J=8.7, 7.3, 1.6 Hz, 1H), 7.44 (s, 1H), 7.362 (s, 1H), 7.360 (br, 1H), 7.29 (ddd, J=8.1, 6.5, 1.5 Hz, 1H), 7.11 (td, J=8.2, 1.6 Hz, 1H), 4.67-4.61 (m, 1H), 4.54-4.51 (m, 2H), 4.50 (dd, J=11.7, 2.4 Hz, 1H), 4.29 (dd, J=11.8, 7.1 Hz, 1H), 3.13 (s, 3H).

### (7S)-N-(3-Bromo-2-fluorophenyl)-7-[(4-methylpiperazin-1-yl)methyl]-7,8-dihydro[1,4]dioxino[2,3-g]quinazolin-4-amine (JGK068S)

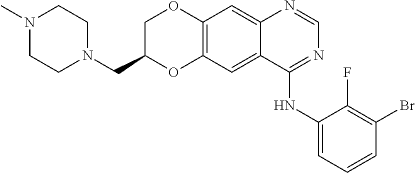 [SMILES: CN1CCN(C[C@H]2COc3cc4ncnc(Nc5cccc(Br)c5F)c4cc3O2)CC1]

A mixture of (R)-9 in DMF (427 mL) was treated with 1-methylpiperazine (11.83 mL, 107 mmol) and Et₃N (5.95 mL, 42.7 mmol), and the mixture was stirred at 85° C. for 24 h. The mixture was cooled to 23° C., and evaporated. The residue was dissolved in EtOAc (1.2 L), and washed with 0.5 M NaOH (4×250 mL), brine (250 mL), dried (Na₂SO₄), filtered, and evaporated. Purification by flash chromatography (CH₂Cl₂/MeOH 1:0→8:2) afforded the title compound JGK068S (6.013 g, 58% over two steps) as an off-white, friable foam. ¹H NMR (500 MHz, CDCl₃): δ8.67 (s, 1H), 8.63 (ddd, J=8.7, 7.3, 1.6 Hz, 1H), 7.374 (s, 1H), 7.372 (br, 1H), 7.32 (s, 1H), 7.26 (ddd, J=8.1, 6.5, 1.5 Hz, 1H), 7.09 (td, J=8.2, 1.6 Hz, 1H), 4.48-4.40 (m, 2H), 4.14 (dd, J=11.8, 8.0 Hz, 1H), 2.77 (dd, J=13.4, 6.0 Hz, 1H), 2.653 (dd, J=13.4, 5.8 Hz, 1H), 2.648 (br, 4H), 2.51 (br, 4H), 2.32 (s, 3H).

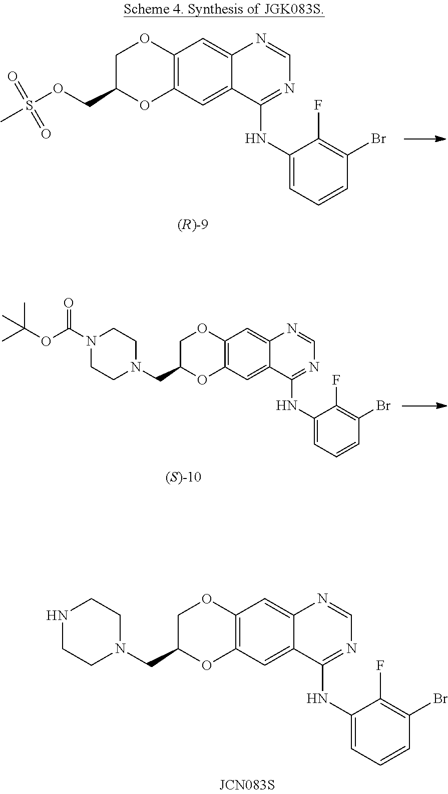 [SMILES: CS(=O)(=O)OC[C@H]1COc2cc3ncnc(Nc4cccc(Br)c4F)c3cc2O1 ; CC(C)(C)OC(=O)N1CCN(C[C@H]2COc3cc4ncnc(Nc5cccc(Br)c5F)c4cc3O2)CC1 ; Fc1c(Br)cccc1Nc1ncnc2cc3c(cc12)O[C@@H](CN1CCNCC1)CO3]

### tert-Butyl{[7S)-4-(3-bromo-2-fluoroanilino)-7,8-dihydro[1,4]dioxino[2,3-g]quinazolin-7-yl]methyl}piperazine-1-carboxylate ((S)-10)

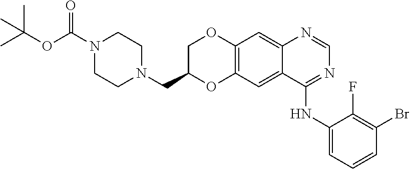 [SMILES: CC(C)(C)OC(=O)N1CCN(C[C@H]2COc3cc4ncnc(Nc5cccc(Br)c5F)c4cc3O2)CC1]

Following general procedure GP-1 of Example 16, compound (S)-10 was prepared from R-9 (91 mg, 0.188 mmol) and tert-butyl piperazine-1-carboxylate (175 mg, 0.94 mmol) in DMF (3.8 mL), and stirred at 85° C. for 15 h. PTLC (CH₂Cl₂/EtOAc 4:6) afforded (S)-10 (50 mg, 46%) as an off-white, friable foam. ¹H NMR (500 MHz, CDCl₃): δ8.68 (s, 1H), 8.65 (ddd, J=8.3, 7.4, 1.5 Hz, 1H), 7.39 (s, 1H), 7.36 (br, 1H), 7.31 (s, 1H), 7.27 (ddd, J=8.0, 6.5, 1.5 Hz, 1H), 7.11 (td, J=8.2, 1.5 Hz, 1H), 4.50-4.42 (m, 2H), 4.17 (dd, J=12.1, 8.2 Hz, 1H), 3.47 (t, J=5.1 Hz, 4H), 2.78 (dd, J=13.4, 5.9 Hz, 1H), 2.67 (dd, J=13.5, 5.9 Hz, 1H), 2.62-2.47 (m, 4H), 1.47 (s, 9H). ¹³C NMR (126 MHz, CDCl₃): δ155.89, 154.83, 153.39, 150.15 (d, J_{CF}=242.4 Hz), 149.36, 146.72, 144.02, 128.63 (d, J_{CF}=10.3 Hz), 127.27, 125.34, 121.78, 114.34, 110.66, 108.60 (d, J_{CF}=19.5 Hz), 106.06, 79.97, 71.76, 67.18, 58.56, 53.96, 28.57, one carbon signal missing (probably due to overlapping peaks). HRMS (DART): m/z [M+H]⁺ calcd for C₂₆H₃₀BrFN₅O₄⁺, 574.1460; found, 574.1432.

### (7S)-N-(3-Bromo-2-fluorophenyl)-7-[(piperazin-1-yl)methyl]-7,8-dihydro[1,4]dioxino [2,3-g]quinazolin-4-amine (JGK083S)

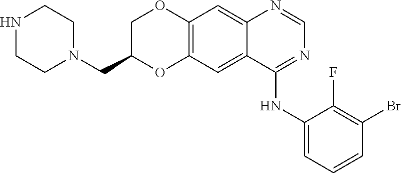 [SMILES: Fc1c(Br)cccc1Nc1ncnc2cc3c(cc12)O[C@@H](CN1CCNCC1)CO3]

A mixture of (S)-10 (42 mg, 0.073 mmol) in CH₂Cl₂ (500 μL) and TFA (250 μL) was stirred at 23° C. for 12 h. The mixture was diluted with 1 M HCl (20 mL), and washed with CH₂Cl₂ (3×7 mL). The aqueous phase was diluted with 6 M NaOH (4 mL) to pH>12, and extracted with EtOAc (3×8 mL). The combined organics were washed with brine (8 mL), dried (Na₂SO₄), filtered, and evaporated. Purification by PTLC (CH₂CL₂/MeOH 8:2) afforded the title compound JGK083S (18 mg, 52%) as a white, friable foam. ¹H NMR (500 MHz, CDCl₃): δ8.68 (s, 1H), 8.66 (ddd, J=8.2, 7.3, 1.6 Hz, 1H), 7.39 (s, 1H), 7.35 (br d, J=4.0 Hz, 1H), 7.32 (s, 1H), 7.27 (ddd, J=8.1, 6.5, 1.6 Hz, 1H), 7.11 (td, J=8.2, 1.5 Hz, 1H), 4.50-4.42 (m, 2H), 4.19-4.13 (m, 1H), 2.93 (t, J=4.9 Hz, 4H), 2.76 (dd, J=13.4, 5.9 Hz, 1H), 2.63 (dd, J=13.4, 6.0 Hz, 1H), 2.63-2.50 (m, 4H). ¹³C NMR (126 MHz, CDCl₃): δ155.88, 153.35, 150.12 (d, J_{CF}=242.3 Hz), 149.45, 146.71, 144.17, 128.67 (d, J_{CF}=10.4 Hz), 127.21, 125.32 (d, J_{CF}=4.7 Hz), 121.74, 114.30, 110.64, 108.58 (d, J_{CF}=19.3 Hz), 106.02, 71.70, 67.32, 59.19, 55.54, 46.23. HRMS (DART): m/z [M−H]⁻ calcd for C₂₁H₂₀BrFN₅O₂⁻, 472.0790; found, 472.0773.

### [(2R)-7-Formyl-2,3-dihydro-1,4-benzodioxin-2-yl]methyl acetate ((R)-10)

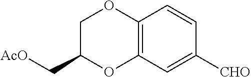 [SMILES: CC(=O)OC[C@H]1COc2ccc(C=O)cc2O1]

A mixture of 1 (150 mg, 0.833 mmol) and (2R)-glycidyl tosylate (203 mg, 0.891 mmol) in DMF (2 mL) was treated with K₂CO₃ (181 mg, 1.31 mmol), and the mixture was stirred at 60° C. for 15 h. The mixture was cooled to 23° C., water (30 mL) was added, and the mixture was extracted with EtOAc (3×15 mL). The combined organics were washed with water (15 mL), brine (15 mL), dried (Na₂SO₄), filtered, and evaporated. Purification by preparative thin layer chromatography (hexanes/EtOAc 7:3) afforded the title compound (R)-10 (111 mg, 56%) as a clear, colorless oil. ¹H NMR (400 MHz, CDCl₃): δ=9.82 (s, 1H), 7.44 (d, J=1.8 Hz, 1H), 7.42 (dd, J=8.2, 1.9 Hz, 1H), 7.00 (d, J=8.1 Hz, 1H), 4.46-4.39 (m, 1H), 4.37 (dd, J=11.5, 2.4 Hz, 1H), 4.35 (dd, J=11.7, 4.9 Hz, 1H), 4.31 (dd, J=11.9, 5.1 Hz, 1H), 4.13 (dd, J=11.5, 7.1 Hz, 1H), 2.11 (s, 3H). ¹³C NMR (101 MHz, CDCl₃): δ190.72, 170.67, 148.57, 143.22, 131.15, 124.38, 118.76, 117.85, 70.94, 65.54, 62.36, 20.83. HRMS (DART): m/z [M+H]⁺ calcd for C₁₂H₁₃O₅⁺, 237.0757; found, 237.0745.

### (±)-N-(3-Chloro-2-fluorophenyl)-7-[(4-methylpiperazin-1-yl)methyl]-7,8-dihydro[1,4]dioxino[2,3-g]quinazolin-4-amine (JGK075)

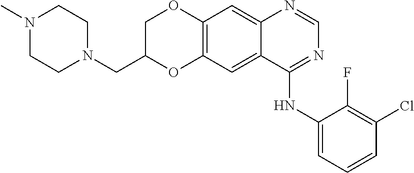 [SMILES: CN1CCN(CC2COc3cc4ncnc(Nc5cccc(Cl)c5F)c4cc3O2)CC1]

¹H NMR (500 MHz, CDCl₃): δ8.68 (s, 1H), 8.61 (td, J=7.3, 2.2 Hz, 1H), 7.39 (s, 1H), 7.35 (br d, J=3.4 Hz, 1H), 7.32 (s, 1H), 7.16 (td, J=8.1, 1.2 Hz, 1H), 7.13 (td, J=8.2, 1.9 Hz, 1H), 4.49-4.41 (m, 2H), 4.15 (dd, J=11.8, 8.1 Hz, 1H), 2.78 (dd, J=13.4, 5.9 Hz, 1H), 2.66 (dd, J=13.4, 5.9 Hz, 1H), 2.64 (br, 4H), 2.48 (br, 4H), 2.31 (s, 3H). ¹³C NMR (126 MHz, CDCl₃): δ155.89, 153.35, 149.44, 149.30 (d, J_{CF}=244.2 Hz), 146.72, 144.15, 128.76 (d, J_{CF}=9.3 Hz), 124.71 (d, J_{CF}=4.7 Hz), 124.45, 121.01, 120.85 (d, J_{CF}=16.1 Hz), 114.30, 110.63, 106.05, 71.81, 67.31, 58.50, 55.17, 54.15, 46.19. HRMS (DART): m/z [M+H]⁺ calcd for C₂₂H₂₄ClFN₅O₂⁺, 444.1597; found, 444.1582.

### (±)-N-(3-Bromo-2-fluorophenyl)-8-[(morpholin-4-yl)methyl]-7,8-dihydro[1,4]dioxino[2,3-g]quinazolin-4-amine (JGK076)

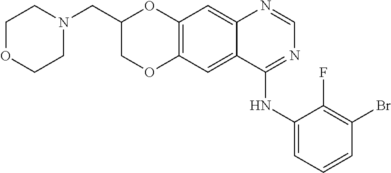 [SMILES: Fc1c(Br)cccc1Nc1ncnc2cc3c(cc12)OCC(CN1CCOCC1)O3]

¹H NMR (500 MHz, DMSO-d₆): δ9.62 (s, 1H), 8.33 (s, 1H), 7.94 (s, 1H), 7.59 (ddd, J=8.0, 6.2, 1.6 Hz, 1H), 7.54 (ddd, J=8.5, 7.1, 1.6 Hz, 1H), 7.21 (td, J=8.1, 1.2 Hz, 1H), 7.19 (s, 1H), 4.63-4.56 (m, 1H), 4.46 (dd, J=11.6, 2.5 Hz, 1H), 4.17 (dd, J=11.6, 7.1 Hz, 1H), 3.59 (t, J=4.6 Hz, 4H), 2.71-2.59 (m, 2H), 2.57-2.44 (m, 4H). ¹³C NMR (126 MHz, DMSO-d₆): δ157.22, 153.37 (d, J_{CF}=247.3 Hz), 153.13, 148.75, 146.16, 143.28, 130.14, 128.02 (d, J_{CF}=13.0 Hz), 127.74, 125.45 (d, J_{CF}=4.7 Hz), 112.57, 109.61, 108.55 (d, J_{CF}=19.9 Hz), 108.23, 71.41, 66.29, 66.18, 57.97, 53.89. HRMS (DART): m/z [M−H]⁻ calcd for C₂₁H₁₉BrFN₄O₃⁻, 473.0630; found, 473.0608.

### (±)-N-(3-Bromo-2-fluorophenyl)-8-[(dimethylamino)methyl]-7,8-dihydro [1,4]dioxino[2,3-g]quinazolin-4-amine (JGK077)

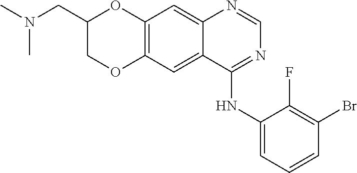 [SMILES: CN(C)CC1COc2cc3c(Nc4cccc(Br)c4F)ncnc3cc2O1]

¹H NMR (500 MHz, DMSO-d₆): δ9.62 (s, 1H), 8.33 (s, 1H), 7.94 (s, 1H), 7.59 (ddd, J=8.0, 6.2, 1.6 Hz, 1H), 7.54 (ddd, J=8.5, 7.1, 1.6 Hz, 1H), 7.21 (td, J=8.1, 1.2 Hz, 1H), 7.17 (s, 1H), 4.57-4.51 (m, 1H), 4.44 (dd, J=11.6, 2.5 Hz, 1H), 4.14 (dd, J=11.7, 7.1 Hz, 1H), 2.58 (s, 1H), 2.57 (s, 1H), 2.25 (s, 6H). ¹³C NMR (126 MHz, DMSO-d₆): δ157.22, 153.38 (d, J_{CF}=247.4 Hz), 153.12, 148.78, 146.16, 143.29, 130.14, 128.02 (d, J_{CF}=13.1 Hz), 127.75, 125.45 (d, J_{CF}=4.4 Hz), 112.54, 109.59, 108.55 (d, J_{CF}=19.8 Hz), 108.20, 71.76, 66.31, 58.73, 45.92. HRMS (DART): m/z [M−H]⁻ calcd for C₁₉H₁₇BrFN₄O₂⁻, 431.0524; found, 431.0503.

### (±)-N-(3-Bromo-2-fluorophenyl)-8- [(4-methylpiperazin-1-yl)methyl]-7,8-dihydro[1,4]dioxino[2,3-g]quinazolin-4-amine (JGK078)

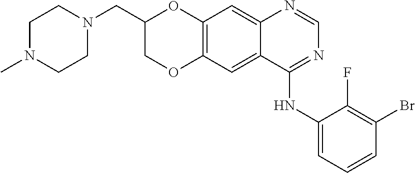 [SMILES: CN1CCN(CC2COc3cc4c(Nc5cccc(Br)c5F)ncnc4cc3O2)CC1]

¹ H NMR (500 MHz, DMSO-d₆): δ9.61 (s, 1H), 8.33 (s, 1H), 7.94 (s, 1H), 7.59 (ddd, J=8.0, 6.2, 1.6 Hz, 1H), 7.54 (ddd, J=8.5, 7.1, 1.6 Hz, 1H), 7.21 (td, J=8.1, 1.2 Hz, 1H), 7.19 (s, 1H), 4.60-4.53 (m, 1H), 4.44 (dd, J=11.6, 2.5 Hz, 1H), 4.14 (dd, J=11.7, 7.1 Hz, 1H), 2.68-2.59 (m, 2H), 2.53 (br, 4H), 2.34 (br, 4H), 2.16 (s, 3H). ¹³C NMR (126 MHz, DMSO-d₆): δ157.22, 153.38 (d, J_{CF}=247.4 Hz), 153.12, 148.78, 146.15, 143.29, 130.13, 128.02 (d, J_{CF}=13.1 Hz), 127.74, 125.45 (d, J_{CF}=4.5 Hz), 112.55, 109.60, 108.55 (d, J_{CF}=19.8 Hz), 108.22, 71.57, 66.34, 57.52, 54.68, 53.29, 45.72. HRMS (DART): m/z [M−H]⁻ calcd for C₂₂H₂₂BrFN₅O₂⁻, 486.0946; found, 486.0928.

### (±)-N-(3-Bromo-2-fluorophenyl)-8-[(pyrrolidin-1-yl)methyl]-7,8-dihydro [1,4]dioxino[2,3-g]quinazolin-4-amine (JGK079)

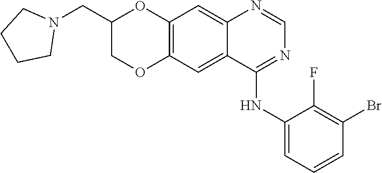 [SMILES: Fc1c(Br)cccc1Nc1ncnc2cc3c(cc12)OCC(CN1CCCC1)O3]

¹H NMR (500 MHz, DMSO-d₆): δ9.61 (s, 1H), 8.33 (s, 1H), 7.94 (s, 1H), 7.59 (ddd, J=8.0, 6.3, 1.6 Hz, 1H), 7.54 (ddd, J=8.5, 7.1, 1.6 Hz, 1H), 7.21 (td, J=8.0, 1.2 Hz, 1H), 7.19 (s, 1H), 4.57-4.49 (m, 1H), 4.46 (dd, J=11.6, 2.5 Hz, 1H), 4.16 (dd, J=11.6, 7.1 Hz, 1H), 2.80 (dd, J=12.8, 6.0 Hz, 1H), 2.73 (dd, J=12.8, 6.2 Hz, 1H), 2.62-2.48 (m, 4H), 1.74-1.66 (m, 4H). ¹³C NMR (126 MHz, DMSO-d₆): δ157.22, 153.37 (d, J_{CF}=247.5 Hz), 153.12, 148.79, 146.17, 143.29, 130.13, 128.02 (d, J_{CF}=12.9 Hz), 127.74, 125.45 (d, J_{CF}=4.5 Hz), 112.54, 109.58, 108.55 (d, J_{CF}=20.0 Hz), 108.19, 72.65, 66.32, 55.42, 54.31, 23.23. HRMS (DART): m/z [M−H]⁻ calcd for C₂₁H₁₉BrFN₄O₂⁻, 457.0681; found, 457.0660.

### (±)-N-(3-Bromo-2-fluorophenyl)-8-[(piperidin-1-yl)methyl]-7,8-dihydro[1,4]dioxino[2,3-g]quinazolin-4-amine (JGK080)

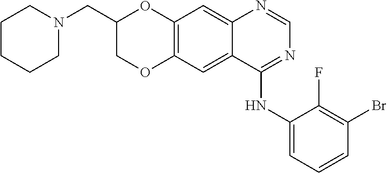 [SMILES: Fc1c(Br)cccc1Nc1ncnc2cc3c(cc12)OCC(CN1CCCCC1)O3]

¹H NMR (500 MHz, DMSO-d₆): δ9.61 (s, 1H), 8.33 (s, 1H), 7.94 (s, 1H), 7.59 (ddd, J=8.0, 6.3, 1.6 Hz, 1H), 7.54 (ddd, J=8.4, 7.0, 1.6 Hz, 1H), 7.21 (td, J=8.1, 1.2 Hz, 1H), 7.18 (s, 1H), 4.59-4.52 (m, 1H), 4.44 (dd, J=11.6, 2.5 Hz, 1H), 4.14 (dd, J=11.7, 7.1 Hz, 1H), 2.65-2.54 (m, 2H), 2.53-2.37 (m, 4H), 1.55-1.47 (m, 4H), 1.43-1.34 (m, 2H). ¹³C NMR (126 MHz, DMSO-d₆): δ157.21, 153.37 (d, J_{CF}=247.1 Hz), 153.11, 148.83, 146.15, 143.32, 130.12, 128.03 (d, J_{CF}=13.1 Hz), 127.73, 125.45 (d, J_{CF}=4.5 Hz), 112.53, 109.57, 108.55 (d, J_{CF}=19.8 Hz), 108.19, 71.63, 66.42, 58.35, 54.74, 25.61, 23.83. HRMS (DART): m/z [M−H]⁻ calcd for C₂₂H₂₁BrFN₄O₂⁻, 471.0837; found, 471.0814.

### N-(3-Bromo-2-fluorophenyl)(7,7,8,8- ²H₄)-7,8-dihydro[1,4]dioxino[2,3-g]quinazolin-4-amine (JGK081)

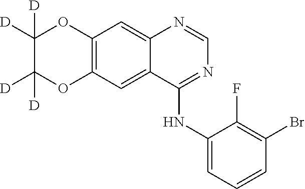 [SMILES: [2H]C1([2H])Oc2cc3ncnc(Nc4cccc(Br)c4F)c3cc2OC1([2H])[2H]]

¹H NMR (500 MHz, DMSO-d₆): δ9.61 (s, 1H), 8.33 (s, 1H), 7.93 (s, 1H), 7.59 (ddd, J=7.9, 6.3, 1.6 Hz, 1H), 7.54 (ddd, J=8.4, 7.1, 1.6 Hz, 1H), 7.21 (td, J=8.1, 1.2 Hz, 1H), 7.19 (s, 1H). ¹³C NMR (126 MHz, DMSO-d₆): δ157.19, 153.37 (d, J_{CF}=247.2 Hz), 153.10, 149.27, 146.03, 143.67, 130.12, 128.03 (d, J_{CF}=13.0 Hz), 127.74, 125.44 (d, J_{CF}=4.2 Hz), 112.47, 109.63, 108.55 (d, J_{CF}=19.9 Hz), 108.35. HRMS (DART): m/z [M+H]⁺ calcd for C₁₆H₈D₄BrFN₃O₂⁺, 380.0342; found, 380.0327.

### (±)-N-(3-Bromo-2-fluorophenyl)-7-[2-(pyrrolidin-1-yl)ethyl]-7,8-dihydro[1,4]dioxino[2,3-g]quinazolin-4-amine (JGK084)

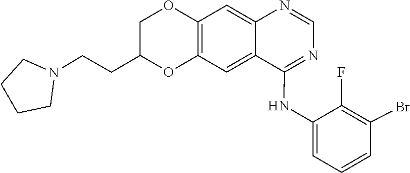 [SMILES: Fc1c(Br)cccc1Nc1ncnc2cc3c(cc12)OC(CCN1CCCC1)CO3]

¹H NMR (500 MHz, DMSO-d₆): δ9.60 (s, 1H), 8.33 (s, 1H), 7.95 (s, 1H), 7.58 (ddd, J=8.0, 6.2, 1.6 Hz, 1H), 7.53 (ddd, J=8.4, 7.0, 1.6 Hz, 1H), 7.21 (td, J=8.1, 1.1 Hz, 2H), 7.20 (s, 1H), 4.50 (dd, J=11.5, 2.3 Hz, 1H), 4.42-4.36 (m, 1H), 4.12 (dd, J=11.5, 7.7 Hz, 1H), 2.70-2.56 (m, 2H), 2.49-2.40 (m, 4H), 1.89-1.78 (m, 2H), 1.73-1.64 (m, 4H). ¹³C NMR (126 MHz, DMSO-d₆): δ157.21, 153.33 (d, J_{CF}=247.5 Hz), 153.13, 148.95, 146.02, 143.37, 130.08, 128.07 (d, J_{CF}=13.1 Hz), 127.64, 125.48 (d, J_{CF}=4.6 Hz), 112.26, 109.78, 108.58 (d, J_{CF}=19.8 Hz), 108.44, 71.76, 67.78, 53.63, 51.03, 29.53, 23.16. HRMS (DART): m/z [M+H]⁺ calcd for C₂₂H₂₃BrFN₄O₂⁺, 473.0983; found, 473.0976.

### (±)-N-(3-Bromo-2-fluorophenyl)-7-[2-(piperidin-1-yl)ethyl]-7,8-dihydro[1,4]dioxino[2,3-g]quinazolin-4-amine (JGK085)

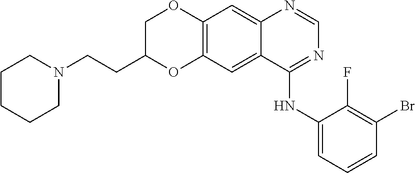 [SMILES: Fc1c(Br)cccc1Nc1ncnc2cc3c(cc12)OC(CCN1CCCCC1)CO3]

¹H NMR (500 MHz, DMSO-d₆): δ9.60 (s, 1H), 8.33 (s, 1H), 7.94 (s, 1H), 7.58 (ddd, J=8.0, 6.2, 1.6 Hz, 1H), 7.53 (ddd, J=8.4, 7.1, 1.6 Hz, 1H), 7.204 (td, J=8.2, 1.3 Hz, 1H), 7.198 (s, 1H), 4.51 (dd, J=11.5, 2.4 Hz, 1H), 4.39-4.33 (m, 1H), 4.11 (dd, J=11.6, 7.8 Hz, 1H), 2.50-2.44 (m, 2H), 2.42-2.27 (m, 4H), 1.90-1.76 (m, 2H), 1.55-1.45 (m, 4H), 1.42-1.34 (m, 2H). ¹³C NMR (126 MHz, DMSO-d₆): δ157.20, 153.33 (d, J_{CF}=247.4 Hz), 153.12, 148.95, 146.02, 143.39, 130.08, 128.07 (d, J_{CF}=13.1 Hz), 127.64, 125.48 (d, J_{CF}=4.5 Hz), 112.25, 109.77, 108.58 (d, J_{CF}=20.0 Hz), 108.43, 71.97, 67.80, 54.08, 53.96, 27.69, 25.61, 24.12. HRMS (DART): m/z [M+H]⁺ calcd for C₂₃H₂₅BrFN₄O₂⁺, 487.1139; found, 487.1137.

### (±)-N-(3-Bromo-2-fluorophenyl)-8-[2-(morpholin-4-yl)ethyl]-7,8-dihydro[1,4]dioxino[2,3-g]quinazolin-4-amine (JGK086)

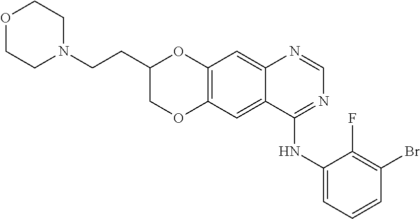 [SMILES: Fc1c(Br)cccc1Nc1ncnc2cc3c(cc12)OCC(CCN1CCOCC1)O3]

¹H NMR (500 MHz, DMSO-d₆): δ9.63 (s, 1H), 8.32 (s, 1H), 7.93 (s, 1H), 7.58 (ddd, J=8.0, 6.3, 1.5 Hz, 1H), 7.53 (t, J=7.0 Hz, 1H), 7.21 (td, J=8.1, 1.2 Hz, 1H), 4.50 (dd, J=11.5, 2.4 Hz, 1H), 4.47-4.40 (m, 1H), 4.10 (dd, J=11.6, 7.4 Hz, 1H), 3.58 (t, J=4.7 Hz, 4H), 2.55-2.46 (m, 2H), 2.45-2.33 (m, 4H), 1.92-1.79 (m, 2H). ¹³C NMR (126 MHz, DMSO-d₆): δ157.20, 153.33 (d, J_{CF}=248.3 Hz), 153.06, 148.94, 146.06, 143.26, 130.03, 128.12 (d, J_{CF}=9.8 Hz), 127.69, 125.44 (d, J_{CF}=4.4 Hz), 112.47, 109.64, 108.55 (d, J_{CF}=19.9 Hz), 108.18, 72.30, 67.35, 66.22, 53.53, 53.28, 27.25. HRMS (DART): m/z [M+H]⁺ calcd for C₂₂H₂₃BrFN₄O₃⁺, 489.0932; found, 489.0926.

### (±)-N-(3-Bromo-2-fluorophenyl)-8-[2-(dimethylamino)ethyl]-7,8-dihydro[1,4]dioxino[2,3-g]quinazolin-4-amine (JGK087)

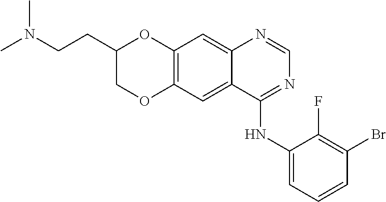 [SMILES: CN(C)CCC1COc2cc3c(Nc4cccc(Br)c4F)ncnc3cc2O1]

¹H NMR (500 MHz, DMSO-d₆): δ9.61 (s, 1H), 8.33 (s, 1H), 7.93 (s, 1H), 7.59 (t, J=6.9 Hz, 1H), 7.54 (t, J=7.5 Hz, 1H), 7.21 (t, J=8.1 Hz, 1H), 7.18 (s, 1H), 4.49 (dd, J=11.6, 2.3 Hz, 1H), 4.45-4.38 (m, 1H), 4.09 (dd, J=11.6, 7.5 Hz, 1H), 2.47-2.38 (m, 2H), 2.17 (s, 6H), 1.86-1.78 (m, 2H). ¹³C NMR (126 MHz, DMSO-d₆): δ157.20, 153.37 (d, J_{CF}=247.6 Hz), 153.08, 148.99, 146.14, 143.29, 130.10, 128.07 (d, J_{CF}=15.6 Hz), 127.72, 125.44 (d, J_{CF}=4.4 Hz), 112.50, 109.58, 108.54 (d, J_{CF}=19.7 Hz), 108.13, 72.30, 67.36, 54.43, 45.17, 28.27. HRMS (DART): m/z [M+H]⁺ calcd for C₂₀H₂₁BrFN₄O₂⁺, 447.0826; found, 447.0818.

### (±)-N-(3-Bromo-2-fluorophenyl)-8-[2-(4-methylpiperazin-1-yl)ethyl]-7,8-dihydro[1,4]dioxino[2,3-g]quinazolin-4-amine (JGK088)

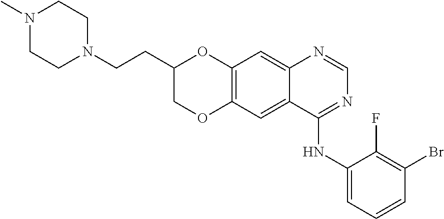 [SMILES: CN1CCN(CCC2COc3cc4c(Nc5cccc(Br)c5F)ncnc4cc3O2)CC1]

¹H NMR (500 MHz, DMSO-d₆): δ9.62 (s, 1H), 8.33 (s, 1H), 7.93 (s, 1H), 7.59 (t, J=7.1 Hz, 1H), 7.54 (t, J=7.5 Hz, 1H), 7.21 (t, J=8.0 Hz, 1H), 7.17 (s, 1H), 4.49 (dd, J=11.5, 2.4 Hz, 1H), 4.45-4.38 (m, 1H), 4.10 (dd, J=11.6, 7.4 Hz, 1H), 2.48-2.21 (m, 10H), 2.14 (s, 3H), 1.91-1.76 (m, 2H). ¹³C NMR (126 MHz, DMSO-d₆): δ157.20, 153.36 (d, J_{CF}=246.9 Hz), 153.08, 148.98, 146.13, 143.28, 130.09, 128.05 (d, J_{CF}=11.7 Hz), 127.72, 125.44 (d, J_{CF}=4.3 Hz), 112.50, 109.58, 108.55 (d, J_{CF}=19.8 Hz), 108.13, 72.40, 67.37, 54.78, 53.11, 52.65, 45.76, 27.61. HRMS (DART): m/z [M+H]⁺ calcd for C₂₃H₂₆BrFN₅O₂⁺, 502.1248; found, 502.1240.

### (±)-N-(3-Bromo-2-fluorophenyl)-8-[2-(pyrrolidin-1-yl)ethyl]-7,8-dihydro [1,4]dioxino[2,3-g]quinazolin-4-amine (JGK089)

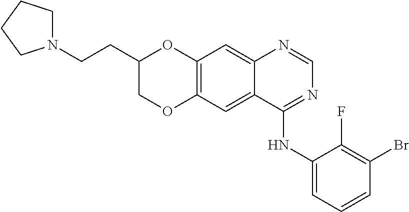 [SMILES: Fc1c(Br)cccc1Nc1ncnc2cc3c(cc12)OCC(CCN1CCCC1)O3]

¹H NMR (500 MHz, DMSO-d₆): δ9.62 (s, 1H), 8.33 (s, 1H), 7.93 (s, 1H), 7.59 (t, J=7.2 Hz, 1H), 7.53 (t, J=7.5 Hz, 1H), 7.21 (t, J=8.0 Hz, 1H), 7.17 (s, 1H), 4.49 (dd, J=11.5, 2.4 Hz, 1H), 4.47-4.41 (m, 1H), 4.10 (dd, J=11.5, 7.4 Hz, 1H), 2.68-2.53 (m, 2H), 2.50-2.40 (m, 4H), 1.89-1.81 (m, 2H), 1.73-1.65 (m, 4H). ¹³C NMR (126 MHz, DMSO-d₆): δ157.20, 153.36 (d, J_{CF}=246.9 Hz), 153.07, 148.98, 146.13, 143.28, 130.07, 128.10, 127.71, 125.44 (d, J_{CF}=4.6 Hz), 112.48, 109.60, 108.55 (d, J_{CF}=19.8 Hz), 108.15, 72.31, 67.37, 53.57, 50.97, 29.58, 23.14. HRMS (DART): m/z [M+H]⁺ calcd for C₂₂H₂₃BrFN₄O₂⁺, 473.0983; found, 473.0976.

### (±)-N-(3-Bromo-2-fluorophenyl)-8-[2-(piperidin-1-yl)ethyl]-7,8-dihydro[1,4]dioxino[2,3-g]quinazolin-4-amine (JGK090)

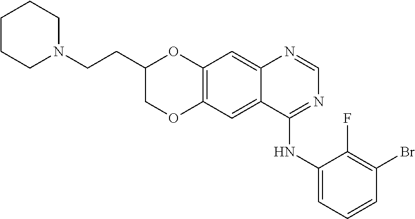 [SMILES: Fc1c(Br)cccc1Nc1ncnc2cc3c(cc12)OCC(CCN1CCCCC1)O3]

¹H NMR (500 MHz, DMSO-d₆): δ9.62 (s, 1H), 8.32 (s, 1H), 7.93 (s, 1H), 7.59 (t, J=7.1 Hz, 1H), 7.53 (t, J=7.5 Hz, 1H), 7.21 (td, J=8.0, 1.2 Hz, 1H), 7.17 (s, 1H), 4.49 (dd, J=11.5, 2.4 Hz, 1H), 4.44-4.37 (m, 1H), 4.10 (dd, J=11.6, 7.4 Hz, 1H), 2.48-2.43 (m, 2H), 2.41-2.27 (m, 4H), 1.90-1.77 (m, 2H), 1.54-1.45 (m, 4H), 1.42-1.34 (m, 2H). ¹³C NMR (126 MHz, DMSO-d₆): δ157.19, 153.34 (d, J_{CF}=246.7 Hz), 153.07, 149.00, 146.11, 143.29, 130.07, 128.10, 127.71, 125.44 (d, J_{CF}=4.3 Hz), 112.48, 109.60, 108.55 (d, J_{CF}=19.9 Hz), 108.14, 72.50, 67.40, 54.02, 53.87, 27.70, 25.63, 24.13. HRMS (DART): m/z [M+H]⁺ calcd for C₂₃H₂₅BrFN₄O₂⁺, 487.1139; found, 487.1133.

### N-(3-Bromo-2-fluorophenyl)-8,9-dihydro-7H-[1,4]dioxepino[2,3-g]quinazolin-4-amine (JGK091)

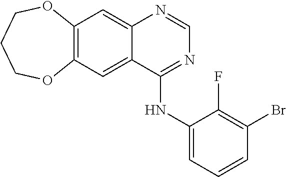 [SMILES: Fc1c(Br)cccc1Nc1ncnc2cc3c(cc12)OCCCO3]

¹H NMR (500 MHz, CDCl₃): δ8.71 (s, 1H), 8.65 (ddd, J=8.3, 7.3, 1.5 Hz, 1H), 7.48 (s, 1H), 7.43 (s, 1H), 7.39 (br, 1H), 7.28 (ddd, J=8.1, 6.5, 1.5 Hz, 1H), 7.11 (td, J=8.2, 1.6 Hz, 1H), 4.41 (t, J=5.7 Hz, 1H), 4.38 (t, J=5.8 Hz, 1H), 2.32 (p, J=5.8 Hz, 1H). ¹³C NMR (126 MHz, CDCl₃): δ157.06, 156.08, 153.93, 151.62, 150.19 (d, J_{CF}=242.7 Hz), 147.83, 128.53 (d, J_{CF}=10.4 Hz), 127.39, 125.32 (d, J_{CF}=4.7 Hz), 121.84, 119.15, 111.47, 110.85, 108.62 (d, J_{CF}=19.3 Hz), 70.86, 70.51, 31.03. HRMS (DART): m/z [M+H]⁺ calcd for C₁₇H₁₄BrFN₃O₂⁺, 390.0248; found, 390.0236.

### N-(3-Bromo-2-fluorophenyl)-2H-[1,3]dioxolo [4,5-g]quinazolin-8-amine (JGK092)

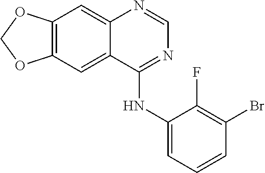 [SMILES: Fc1c(Br)cccc1Nc1ncnc2cc3c(cc12)OCO3]

¹H NMR (500 MHz, CDCl₃): δ8.69 (s, 1H), 8.58 (ddd, J=8.3, 7.3, 1.5 Hz, 1H), 7.28 (ddd, J=8.1, 6.5, 1.6 Hz, 1H), 7.25 (br, 1H), 7.14 (s, 1H), 7.11 (td, J=8.2, 1.6 Hz, 1H), 6.17 (s, 2H), signal of one proton missing (probably hidden by the chloroform signal). ¹³C NMR (126 MHz, CDCl₃): δ156.10, 153.37, 153.22, 150.22 (d, J_{CF}=242.3 Hz), 149.37, 148.43, 128.67 (d, J_{CF}=10.4 Hz), 127.28, 125.30 (d, J_{CF}=4.7 Hz), 121.84, 110.75, 108.64 (d, J_{CF}=19.4 Hz), 106.29, 102.48, 96.49. HRMS (DART): [M+H]⁺ calcd for C₁₅H₁₀BrFN₃O₂⁺, 361.9935; found, 361.9925.

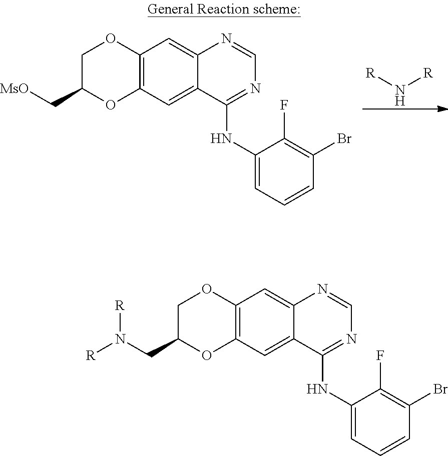

### (7S)-N-(3-bromo-2-fluorophenyl)-7-((1-methylhexahydropyrrolo[3,4-b]pyrrol-5(1H)-yl)methyl)-7,8-dihydro-[1,4]dioxino[2,3-g]quinazolin-4-amine

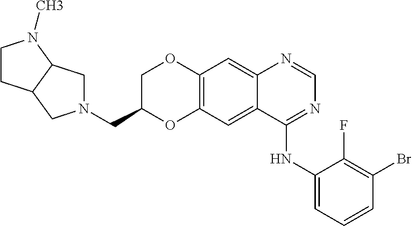 [SMILES: CN1CCC2CN(C[C@H]3COc4cc5ncnc(Nc6cccc(Br)c6F)c5cc4O3)CC21]

To a stirred solution of (R)-(4-((3-bromo-2-fluorophenyl)amino)-7,8-dihydro-[1,4] dioxino[2,3-g]quinazolin-7-yl)methyl methanesulfonate (250 mg, 0.459 mmol) in N,N-dimethylformamide (DMF) (2 mL), was added ′1-methyloctahydropyrrolo[3,4-b]pyrrole (139 mg, 1.09 mmol) followed by the addition of triethylamine (TEA) (0.122 mL, 0.938 mmol). The resulting mixture was heated to 85° C. and allowed to stir overnight, after which it was cooled to room temperature, water was added, and the resulting mixture was extracted with ethyl acetate (EtOAc). The combined organic phases were dried over Na₂SO₄, the mixture was filtered, the filtrate was concentrated. The resulting product was purified by reverse-phase HPLC (RP-HPLC), and the active fractions were combined and concentrated to provide a product that was further extracted with a saturated solution of NaHCO₃ and EtOAc. The combined organic fractions were washed with brine, dried over Na₂SO₄, filtered, and concentrated to give the title compound (62 mg, 27.5%). ¹H NMR (400 MHz, DMSO-d₆): δ9.58 (s; 1H); 8.32 (s; 1H); 7.96 (s; 1H); 7.51-7.59 (m; 2H); 7.19 (t; J=7.98 Hz; 2H); 4.46 (d; J=9.72 Hz; 2H); 4.16 (dd; J=11.81; 7.88 Hz; 1H); 2.56-2.78 (m; 8H); 2.16-2.34 (m; 5H); 1.87 (s; 1H); 1.47 (br s; 1H). MS (ESI): [M+2H]²⁺ Calculated for C₂₄H₂₇BrFN₅O₂²⁺: 257.57, Found: 257.6.

### (7S)-N-(3-bromo-2-fluorophenyl)-7-((5-methylhexahydropyrrolo[3,4-c]pyrrol-2(1H)-yl)methyl)-7,8-dihydro-[1,4]dioxino[2,3-g]quinazolin-4-amine

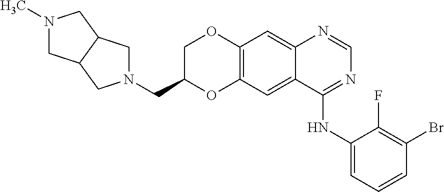 [SMILES: CN1CC2CN(C[C@H]3COc4cc5ncnc(Nc6cccc(Br)c6F)c5cc4O3)CC2C1]

To a stirred solution of (R)-(4-((3-bromo-2-fluorophenyl)amino)-7,8-dihydro-[1,4] dioxino[2,3-g]quinazolin-7-yl)methyl methanesulfonate (250 mg, 0.459 mmol) in DMF (2 mL), added ′2-methyloctahydropyrrolo[3,4-b]pyrrole (139 mg, 1.09 mmol) followed by the addition of TEA (0.122 mL, 0.938 mmol). The resulting mixture was heated to 90° C. and allowed to stir overnight, after which it was cooled to room temperature, water was added, and the resulting mixture was extracted with ethyl acetate (EtOAc). The combined organic phases were dried over Na₂SO₄, the mixture was filtered, the filtrate was concentrated. The resulting product was purified by reverse-phase HPLC (RP-HPLC), and the active fractions were combined and concentrated to provide a product that was further extracted with a saturated solution of NaHCO₃ and EtOAc. The combined organic fractions were washed with brine, dried over Na₂SO₄, filtered, and concentrated to give the title compound (47 mg, 21%). ¹H NMR (400 MHz, DMSO-d₆): δ9.58 (s; 1H); 8.33 (s; 1H); 7.96 (s; 1H); 7.52-7.60 (m; 2H); 7.20-7.22 (m; 2H); 4.47 (d; J=9.95 Hz; 2H); 4.16 (dd; J=11.91; 7.86 Hz; 1H); 2.60-2.78 (m; 6H); 2.20-2.41 (m; 6H); 2.18 (s; 3H). MS (ESI): [M+2H]²⁺ Calculated for C₂₄H₂₇BrFN₅O₂²⁺: 257.57, Found: 257.6.

### (7S)-N-(3-bromo-2-fluorophenyl)-7-((4-methyl-5-(trifluoromethyl)-1,4-diazepan-1-yl)methyl)-7,8-dihydro-[1,4]dioxino[2,3-g]quinazolin-4-amine

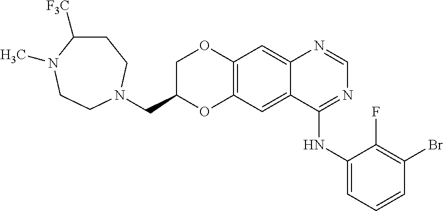 [SMILES: CN1CCN(C[C@H]2COc3cc4ncnc(Nc5cccc(Br)c5F)c4cc3O2)CCC1C(F)(F)F]

To a solution of (R)-(4-((3-bromo-2-fluorophenyl)amino)-7,8-dihydro-[1,4] dioxino[2,3-g]quinazolin-7-yl)methyl methanesulfonate (100 mg, 0.206 mmol) in acetonitrile (MeCN) (1 mL) was added potassium phosphate (350 mg, 1.65 mmol) then 1-methyl-7-(trifluoromethyl)-1,4-diazepane, and trifluoroacetic acid (TFA) (254 mg, 0.62 mmol) and the mixture was heated to reflux and allowed to stir overnight, after which it was cooled to room temperature, water was added, and the resulting mixture was extracted with ethyl acetate (EtOAc). The combined organic phases were dried over Na₂SO₄, the mixture was filtered, the filtrate was concentrated. The resulting product was purified by reverse-phase HPLC (RP-HPLC), and the active fractions were combined and concentrated to provide a product that was further extracted with a saturated solution of NaHCO₃ and EtOAc. The combined organic fractions were washed with brine, dried over MgSO₄, filtered, and concentrated to give the title compound (36 mg, 30%). ¹H NMR (400 MHz, DMSO-d₆): δ9.58 (s; 1H); 8.33 (s; 1H); 7.95 (s; 1H); 7.52-7.60 (m; 2H); 7.20 (t; J=8.05 Hz; 2H); 4.48 (d; J=11.62 Hz; 2H); 4.17 (t; J=9.33 Hz; 1H); 3.03-3.21 (m; 2H); 2.76 (t; J=4.71 Hz; 5H); 2.54-2.58 (m; 4H); 1.83-1.97 (m; 2H); 1.09-1.22 (m; 1H). MS (ESI): m/z [M+2H]²⁺ Calculated for C₂₄H₂₆BrF₄N₅O₂²⁺: 284.55, Found: 285.6.

### (S)-N-(3-bromo-2-fluorophenyl)-7-((6,6-difluoro-4-methyl-1,4-diazepan-1-yl)methyl)-7,8-dihydro-[1,4]dioxino[2,3-g]quinazolin-4-amine

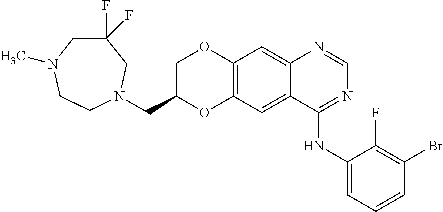 [SMILES: CN1CCN(C[C@H]2COc3cc4ncnc(Nc5cccc(Br)c5F)c4cc3O2)CC(F)(F)C1]

To a solution of (4-((3-bromo-2-fluorophenyl)amino)-7,8-dihydro-[1,4]dioxino[2,3-g]quinazolin-7-yl)methyl methanesulfonate (S-8) (50 mg, 0.10 mmol) in MeCN (0.5 mL) was added potassium phosphate (175 mg, 0.83 mmol), and 6,6-difluoro-1-methyl-1,4-diazepane dihydrochloride (69 mg, 0.31 mmol) and the resulting mixture was heated to reflux and allowed to stir overnight, after which it was cooled to room temperature, water was added, and the resulting mixture was extracted with ethyl acetate (EtOAc). The combined organic phases were dried over Na₂SO₄, the mixture was filtered, the filtrate was concentrated. The resulting product was purified by reverse-phase HPLC (RP-HPLC), and the active fractions were combined and concentrated to provide a product that was further extracted with a saturated solution of NaHCO₃ and EtOAc. The combined organic fractions were washed with brine, dried over MgSO₄, filtered, and concentrated to give the title compound (2 mg, 3.6%). ¹H NMR (400 MHz, CDCl₃): δ8.69-8.62 (m, 2H), 7.41 (s, 1H), 7.29-7.26 (m, 2H), 7.11 (t, 1H, J=9.0 Hz), 4.48 (d, 1H, J=11.5 Hz), 4.37-4.34 (m, 1H), 4.23-4.19 (m, 1H), 3.23 (t, 2H, J=14.1 Hz), 3.08-2.87 (m, 8H), 2.70 (t, 1H, J=5.3 Hz), 2.46 (s, 3H). MS (ESI): m/z [M+2H]²⁺ calcd for C₂₃H₂₅BrF₃N₅O₂²⁺: 269.56, found: 269.60.

### (S)-N-(3-bromo-2-fluorophenyl)-7-((1-methyl-1,6-diazaspiro[3.3]heptan-6-yl)methyl)-7,8-dihydro-[1,4]dioxino[2,3-g]quinazolin-4-amine

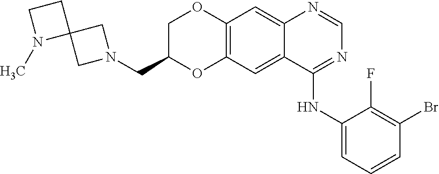 [SMILES: CN1CCC12CN(C[C@H]1COc3cc4ncnc(Nc5cccc(Br)c5F)c4cc3O1)C2]

To a solution of (4-((3-bromo-2-fluorophenyl)amino)-7,8-dihydro-[1,4]dioxino[2,3-g]quinazolin-7-yl)methyl methanesulfonate (250 mg, 0.52 mmol) in MeCN (2.5 mL) was added potassium phosphate (877 mg, 4.13 mmol), and 1-methyl-1,6-diazaspiro[3.3]heptane dihydrochloride (287 mg, 1.55 mmol), and the resulting mixture was heated to reflux and allowed to stir overnight, after which it was cooled to room temperature, water was added, and the resulting mixture was extracted with ethyl acetate (EtOAc). The combined organic phases were dried over Na₂SO₄, the mixture was filtered, the filtrate was concentrated. The resulting product was purified by reverse-phase HPLC (RP-HPLC), and the active fractions were combined and concentrated to provide a product that was further extracted with a saturated solution of NaHCO₃ and EtOAc. The combined organic fractions were washed with brine, dried over MgSO₄, filtered, and concentrated to give the title compound (53 mg, 20.5%). ¹H NMR (400 MHz, DMSO-d₆): δ9.59 (s, 1H), 8.34 (s, 1H), 7.95 (s, 1H), 7.61-7.53 (m, 2H), 7.23-7.19 (m, 2H), 4.42 (d, 1H, J=11.1 Hz), 4.30-4.29 (m, 1H), 4.16-4.11 (m, 1H), 3.39-3.32 (m, 2H), 3.26-3.17 (m, 2H), 2.98 (t, 2H, J=6.4 Hz), 2.74-2.66 (m, 2H), 2.20-2.18 (m, 5H). MS (ESI): m/z [M+2H]²⁺ calcd for C₂₃H₂₅BrFN₅O₂²⁺: 250.56, found: 250.6

### (S)-N-(3-bromo-2-fluorophenyl)-7-((6-methyl-2,6-diazaspiro[3.3]heptan-2-yl)methyl)-7,8-dihydro-[1,4]dioxino[2,3-g]quinazolin-4-amine

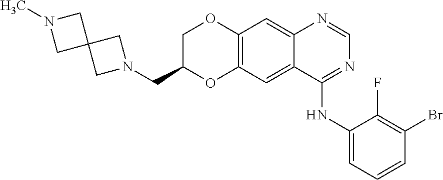 [SMILES: CN1CC2(C1)CN(C[C@H]1COc3cc4ncnc(Nc5cccc(Br)c5F)c4cc3O1)C2]

To a solution of (4-((3-bromo-2-fluorophenyl)amino)-7,8-dihydro-[1,4]dioxino[2,3-g]quinazolin-7-y)methyl methanesulfonate (250 mg, 0.52 mmol) in MeCN (2.5 mL) was added potassium phosphate (877 mg, 4.13 mmol), and 2-methyl-2,6-diazaspiro[3.3]heptane bis(2,2,2-trifluoroacetate) (527 mg, 1.55 mmol) and the and the resulting mixture was extracted with ethyl acetate (EtOAc). The combined organic phases were dried over Na₂SO₄, the mixture was filtered, the filtrate was concentrated. The resulting product was purified by reverse-phase HPLC (RP-HPLC), and the active fractions were combined and concentrated to provide a product that was further extracted with a saturated solution of NaHCO₃ and EtOAc. The combined organic fractions were washed with brine, dried over MgSO₄, filtered, and concentrated to give the title compound (9 mg, 3.5%). ¹H NMR (400 MHz, DMSO-d₆): δ9.59 (s, 1H), 8.34 (s, 1H), 7.95 (s, 1H), 7.61-7.53 (m, 2H), 7.23-7.19 (m, 2H), 4.42-4.39 (m, 1H), 4.29-4.28 (m, 1H), 4.15-4.10 (m, 1H), 3.31-3.26 (m, 4H), 3.20 (s, 4H), 2.72-2.64 (m, 2H), 2.17 (s, 3H). MS (ESI): m/z [M+2H]²⁺ calcd for C₂₃H₂₅BrFN₅O₂²⁺: 250.56, found: 250.6.

### Biological Activity of Exemplary Compounds of the Disclosure

The PC-9 cell line was purchased from Sigma, and the HCC827 cell line was purchased from ATCC. Each cell line was maintained at ≤passage 10 at 37° C. in a humidified incubator with 5% CO₂. Cells were plated in 96-well optical bottom plates (Corning #3903; Corning, NY) at a cell density of 5,000 cells/well, allowed to adhere for minimally 16 hours, and subsequently treated with the test compounds using an 11-point serial dilution (1:3) in duplicate using an HP D300 digital 4 dispenser (Tecan, Morrisville, NC). 72 hours after addition of the test compounds, Cell Titer Glo reagent (Promega, Madison, WI) was added to the plate according to manufacturer's protocol and luminescence measured using a Spectramax M5 (Molecular Devices, San Jose, CA). IC₅₀s were calculated using the 4-parameter variable slope curve fit using the equation below where, Y and X are variables plotted, Top and Bottom are plateaus in the units of the Y axis, LogIC₅₀ is the Log transformation of the IC₅₀ value and HillSlope is the Hill Slope for the curve and describes curve steepness (GraphPad Prism, version 6.07, GraphPad Software, Inc.). Y=Bottom+(Top−Bottom)/(1+10((LogIC50−X)*HillSlope))).

| | Cellular IC₅₀ Data | |
|---|---|---|
| | (EGFR exon 19 deletion) | |
| | PC-9 | HCC827 |
| Compound | IC₅₀ (nM) | IC₅₀ (nM) |
| 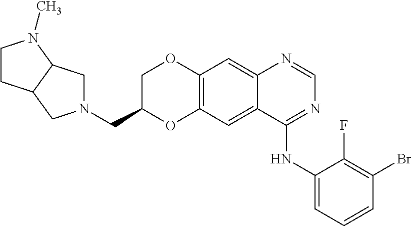 [SMILES: CN1CCC2CN(C[C@H]3COc4cc5ncnc(Nc6cccc(Br)c6F)c5cc4O3)CC21] | 12.9 | 5.9 |
| 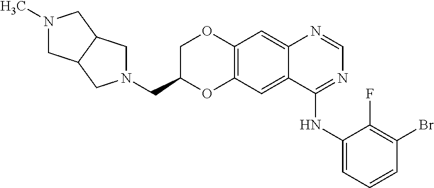 [SMILES: CN1CC2CN(C[C@H]3COc4cc5ncnc(Nc6cccc(Br)c6F)c5cc4O3)CC2C1] | 21.7 | 14.3 |
| 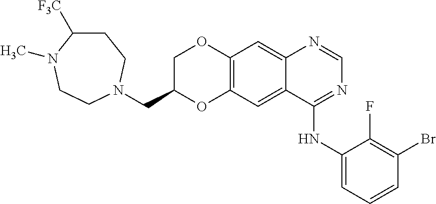 [SMILES: CN1CCN(C[C@H]2COc3cc4ncnc(Nc5cccc(Br)c5F)c4cc3O2)CCC1C(F)(F)F] | 54 | 36.9 |
| 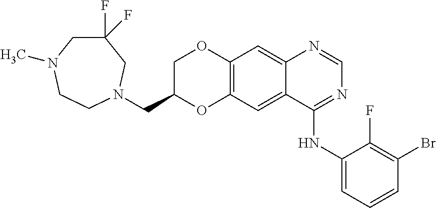 [SMILES: CN1CCN(C[C@H]2COc3cc4ncnc(Nc5cccc(Br)c5F)c4cc3O2)CC(F)(F)C1] | 140 | 50.6 |
| 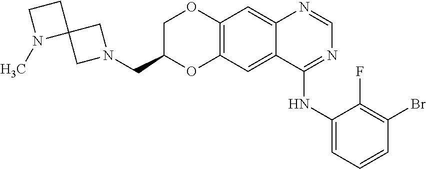 [SMILES: CN1CCC12CN(C[C@H]1COc3cc4ncnc(Nc5cccc(Br)c5F)c4cc3O1)C2] | 9.4 | 7.0 |
| 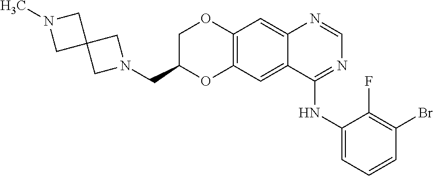 [SMILES: CN1CC2(C1)CN(C[C@H]1COc3cc4ncnc(Nc5cccc(Br)c5F)c4cc3O1)C2] | 10.1 | 5.9 |

### Brain Penetration of Exemplary Compounds of the Disclsoure

Disclosed in table 5 Brain to plasma percentages and unbound ratios of drugs in brain to plasma of indicated drugs in non-tumor bearing mice

TABLE 5
Brain Penetration of Exemplary Compounds of the Disclsoure

| | Brain Penetration | Kpuu |
|---|---|---|
| Compound | (% of plasma) | (Avg) |
| Erlotinib | 8.50 | 0.051 |
| JGK005 | 64.8 | 0.491 |
| JGK038 | 84.3 | 0.575 |
| JGK028 | 106.2 | 1.037 |
| JGK010 | 106.4 | 1.045 |
| JGK037 | 212.1 | 1.301 |
| JGK042 | 167.6 | 1.033 |
| JGK063 | 72.5 | 0.341 |
| JGK066 | 274.3 | 1.175 |
| JGK068 | 354.5 | 1.184 |
| JGK068S | 378.3 | 1.181 |
| JGK074 | 166.2 | n.d. |
| JGK083S | 231.3 | 0.798 |

### Metabolic Studies of Exemplary Compounds of the JGK Series

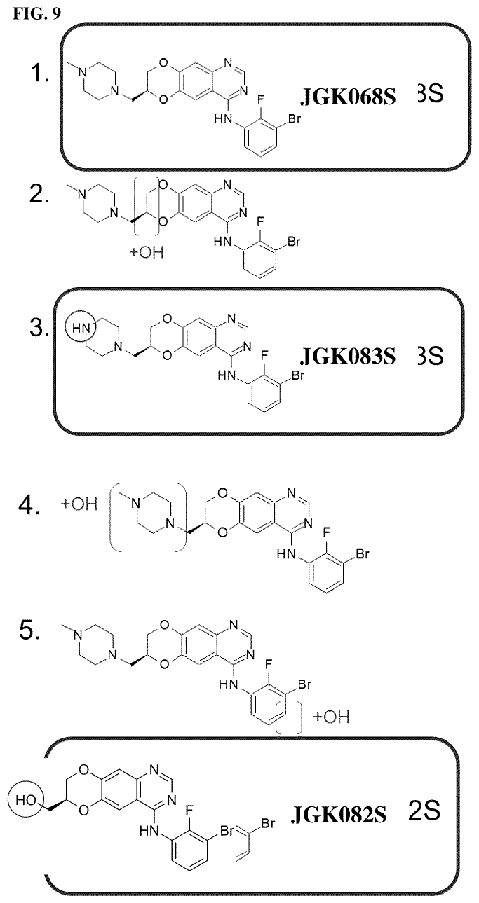

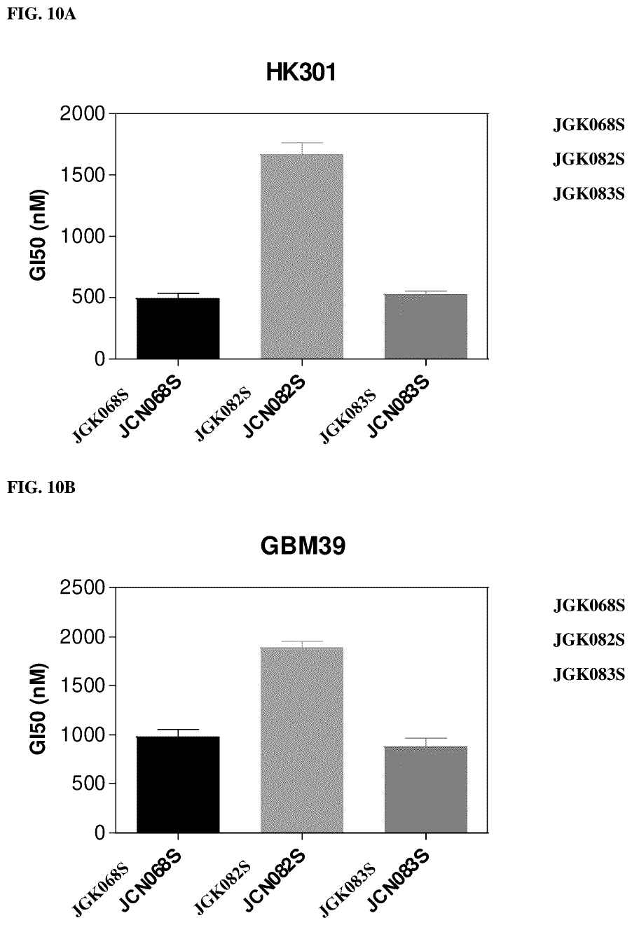

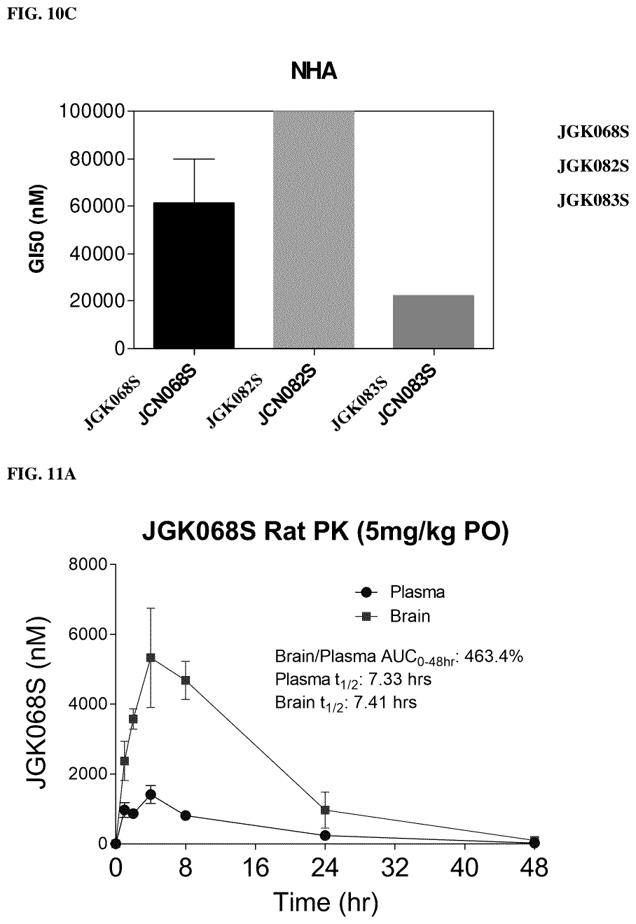

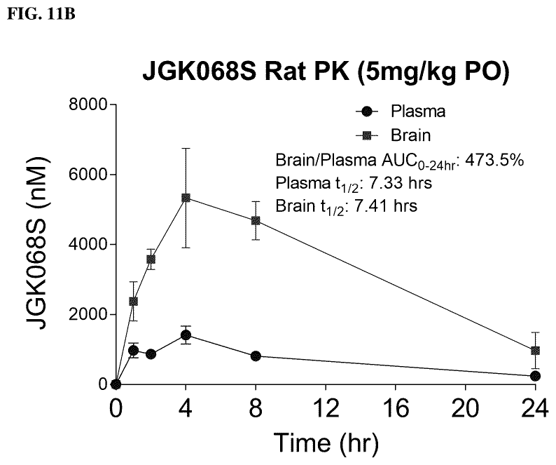

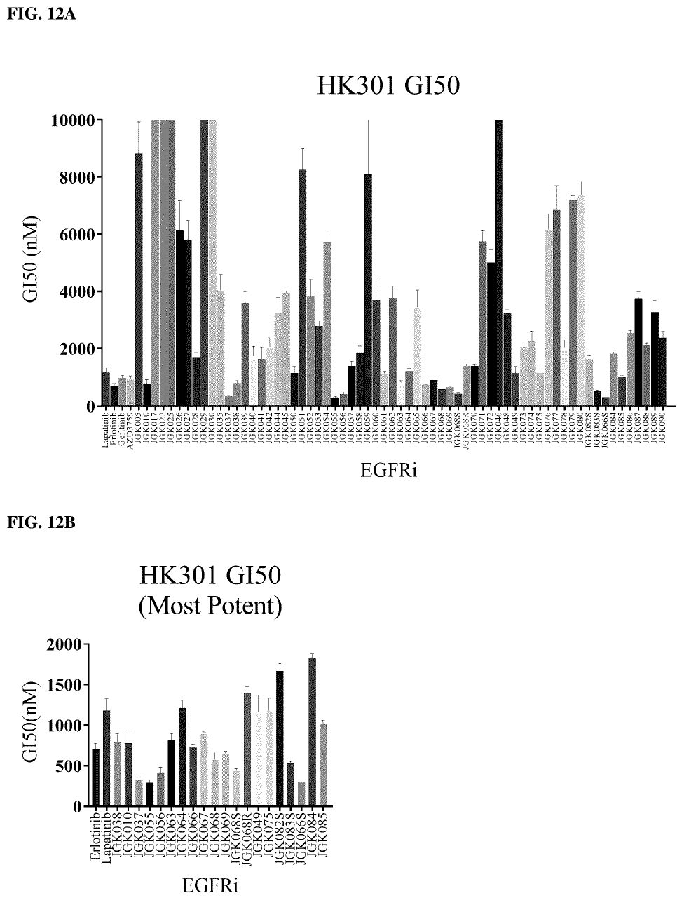

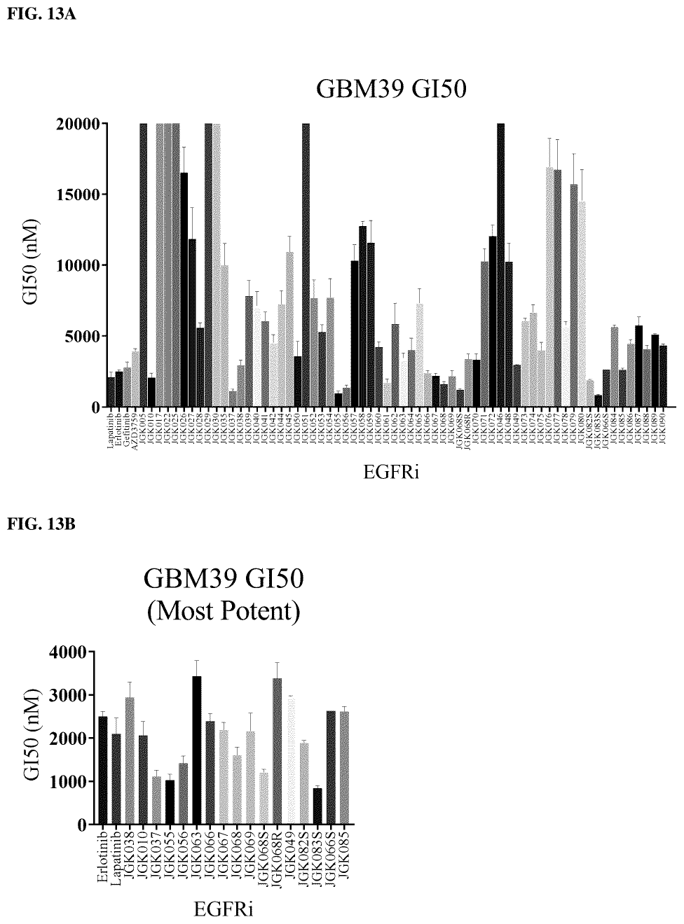

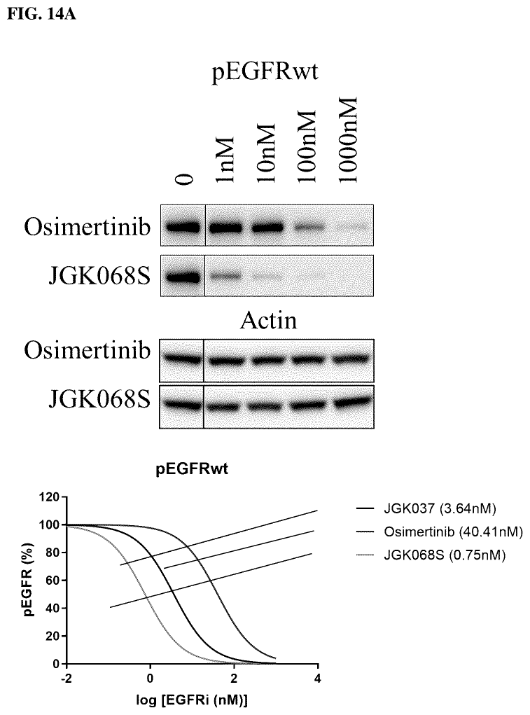

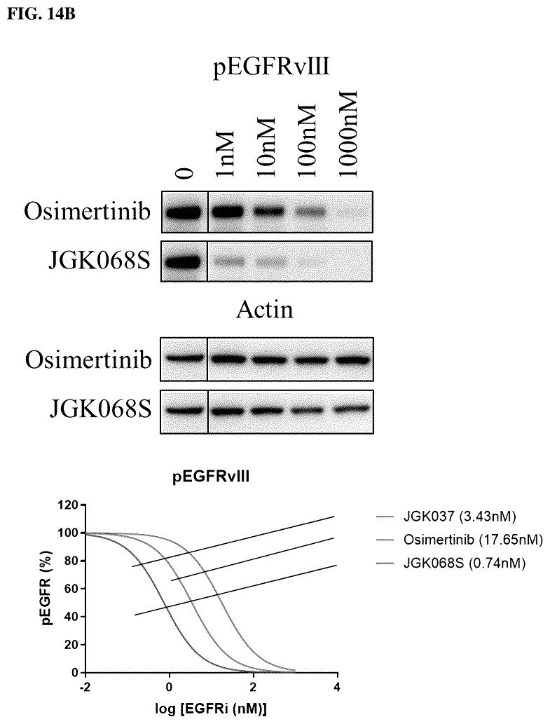

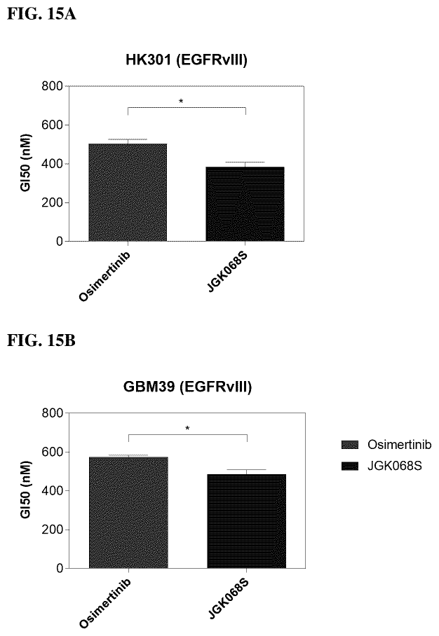

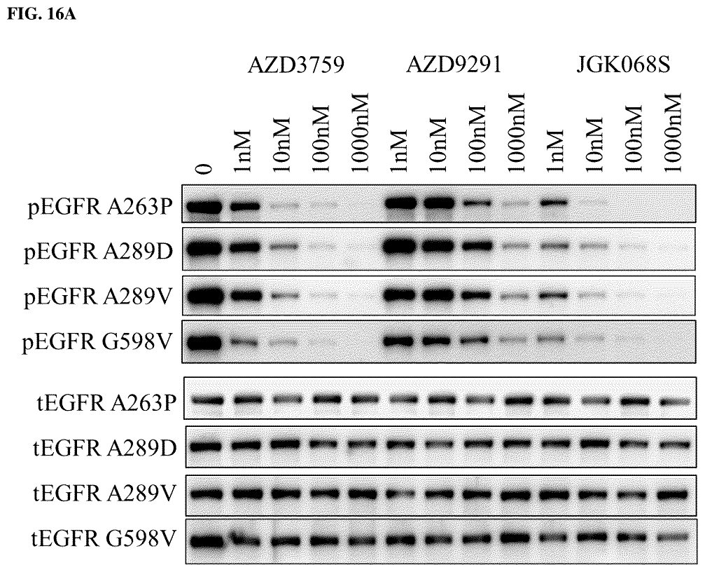

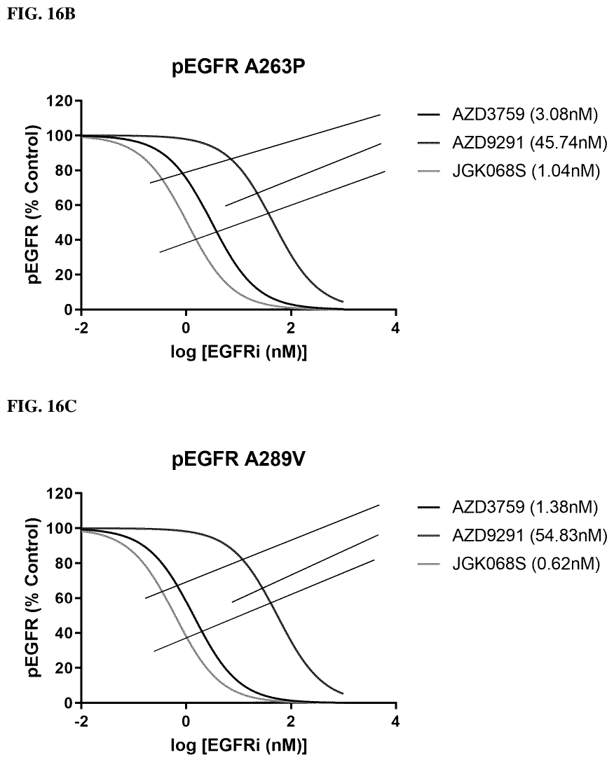

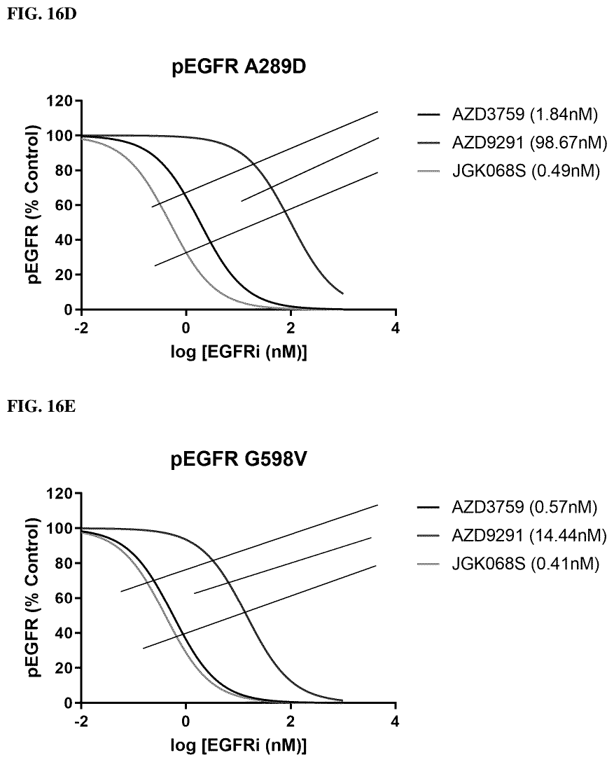

Exemplary compounds (10 mM) were incubated in human, dog, mouse, or rat liver microsomes (1 mg/mL) for up to 90 minutes at 37° C. Reactions were stopped by the addition of acetonitrile. Controls (compound free) microsome studies were run in parallel. LCMS Studies were performed on a Waters Xevo G2 QTof equipped with a Luna Omega Polar C18, 1.6 m, 2.1×30 mm column Strcutures of exmplrary metabolites are decpited in FIG. 9.

| | Human | Dog | Mouse | Rat |
|---|---|---|---|---|
| Modification | (%) | (%) | (%) | (%) |
| 1. Parent | 67.0 | 3.5 | 59.9 | 70.2 |
| 2. Hydroxylation | 6.0 | 0.0 | 0.0 | 4.8 |
| 3. N-demethylation | 13.7 | 0.9 | 5.4 | 8.2 |
| 4. Hydroxylation | 4.2 | 61.9 | 22.0 | 21.9 |
| 5. Hydroxylation | 0.7 | 0.0 | 0.0 | 0.6 |
| 6. N-dealkylation | 6.5 | 0.0 | 1.3 | 2.7 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A compound of Formula (I): 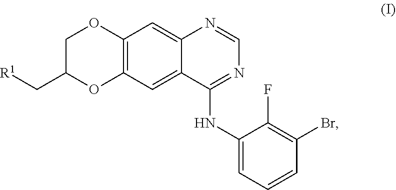
wherein:
R¹ is selected from 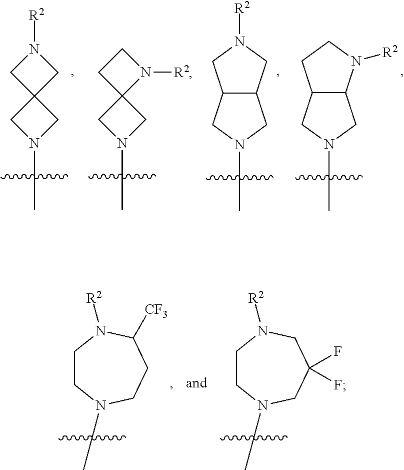
and
R² is selected from C₁-C₆ alkyl and C₃-C₆ cycloalkyl, each of which is optionally substituted with one or more halogen, or a pharmaceutically acceptable salt thereof.

2-7. (canceled)

8. The compound of any one of claim 1, wherein R² is selected from methyl, ethyl, n-propyl, isopropyl, n-butyl, isobutyl, tert-butyl, pentyl, hexyl, trifluoromethyl, fluoroethyl, and difluoroethyl, or a pharmaceutically acceptable salt thereof.

9-10. (canceled)

11. The compound of claim 1, wherein the compound is of Formula (Ia):

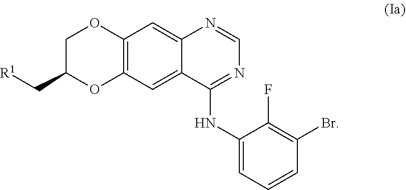

12-20. (canceled)

21. The compound of claim 1, wherein the compound is of Formula (Ib):

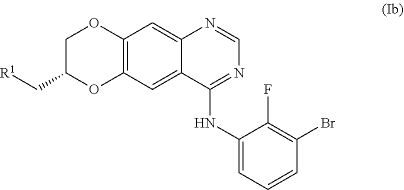

22-30. (canceled)

31. The compound of claim 1, or a pharmaceutically acceptable salt thereof, wherein the compound is enantiomerically enriched.

32. The compound of claim 1, or a pharmaceutically acceptable salt thereof, wherein the compound is diastereomerically enriched.

33. The compound of claim 1, wherein the compound is in the form of a pharmaceutically acceptable salt.

34. The compound of claim 1, wherein the compound is in the form of a free base.

35. A pharmaceutical composition comprising a compound of claim 1, and a pharmaceutically acceptable excipient.

36. A method of treating a disorder or condition in a subject by modulation of an epidermal growth factor receptor comprising administering to the subject, an amount of a compound of claim 1 or a pharmaceutically acceptable salt thereof.

37. The method of claim 36, wherein modulation comprises antagonizing the epidermal growth factor receptor.

38-41. (canceled)

42. The method of claim 36, wherein the disease or disorder is cancer.

43. The method of claim 42, wherein the cancer is bladder cancer, bone cancer, brain cancer, breast cancer, cardiac cancer, cervical cancer, colon cancer, rectal cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, CNS cancer, non-CNS cancer, or CNS metastases.

44-162. (canceled)
